SEQUENCE LISTING (1 sequence)

SEQ ID NO: 1 - protein, 535 aa, synthetic construct
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

(19) United States
(12) Patent Application Publication
(10) Pub. No.: US 2026/0144856 A1
(43) Pub. Date: May 28, 2026

(54) VACCINE AGAINST KLEBSIELLA PNEUMONIAE

(71) Applicant: Idorsia Pharmaceuticals Ltd, Allschwil (CH)

(72) Inventors: Felix BROECKER, Berlin (DE); Claney Lebev PEREIRA, Berlin (DE); Sharavathi Guddehalli PARAMESWARAPPA, Berlin (DE); Arne VON BONIN, Allschwil (CH)

(21) Appl. No.: 19/123,759

(22) PCT Filed: Oct. 24, 2023
(86) PCT No.: PCT/EP2023/079562

§ 371 Date: Apr. 23, 2025

(30) Foreign Application Priority Data
Oct. 24, 2022 (WO) PCT/EP2022/079674
Dec. 9, 2022 (WO) PCT/EP2022/085160

(51) Int. Cl. A61K 39/108; A61K 39/00; A61K 39/385; A61K 47/64; A61P 31/04

(52) U.S. Cl. CPC A61K 39/0266; A61K 39/385; A61K 47/6415; A61K 47/646; A61P 31/04; A61K 2039/575; A61K 2039/6037; A61K 2039/70

(57) ABSTRACT

The present invention relates to novel oligosaccharide-carrier protein conjugates of Formula (I), and their use as pharmaceuticals, in particular as vaccines. The invention also concerns related aspects including oligosaccharide intermediates of Formulae (II) and (III), as well as processes for the preparation of the conjugates. Furthermore, the invention relates to pharmaceutical compositions comprising the oligosaccharide-carrier protein conjugates, as well as the use of the oligosaccharide-carrier protein conjugates of Formula (IV) in biological assays.

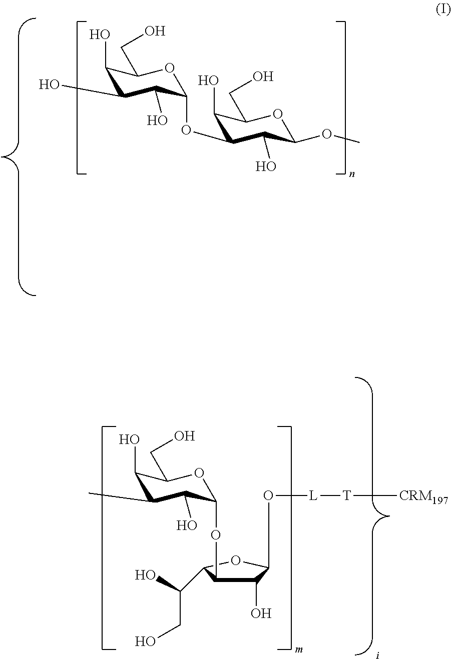

This invention was made with United States government support under IDSEP160030-01, IDSEP160030-02 and IDSEP160030-03 awarded by HHS/ASPR. The United States government has certain rights in the invention.

The present invention relates to novel oligosaccharide-carrier protein conjugates of Formula (I), and their use as pharmaceuticals, in particular as vaccines. The invention also concerns related aspects including oligosaccharide intermediates of Formulae (II) and (III), as well as processes for the preparation of the conjugates. Furthermore, the invention relates to pharmaceutical compositions comprising the oligosaccharide-carrier protein conjugates, as well as the use of the oligosaccharide-carrier protein conjugates of Formula (IV) in biological assays.

Klebsiella pneumoniae (or K. pneumoniae) is a gram-negative, facultative anaerobic, rod-shaped bacterium colonizing mainly respiratory, intestinal and urinary tracts as well as the skin and causing K. pneumoniae infections (KPIs). The bacterium mainly acts as an opportunistic pathogen. KPIs are a major cause of nosocomial infections, primarily affecting immunocompromised patients. Infections caused by K. pneumoniae are an important challenge in healthcare settings due to the emergence of strains resistant to almost all available antimicrobial agents and their worldwide dissemination. Infections caused by K. pneumoniae are responsible for high rates of morbidity and mortality. Thus, prevention of infections caused by K. pneumoniae is highly desirable, and vaccination is the most cost-efficient and the most powerful means to fight KPIs.

K. pneumoniae is an encapsulated bacterium, expressing lipopolysaccharide (LPS) and capsular polysaccharide (CPS, K-antigen) on their outer membrane, which contribute to the virulence of this species.

The LPS consists of three components, namely a lipid A moiety which serves as a membrane anchor, a core oligosaccharide covalently bound to lipid A, and a terminal antigenic polysaccharide comprising repeating saccharide units forming the O-antigen which is covalently bound to the core oligosaccharide. Extracted LPS has been shown to be pyrogenic, toxic and able to cause tissue damage. LPS may be masked by CPS and is usually less exposed to the surface than CPS.

The CPS is comprised of repeating saccharide units that form a layer on the outer bacterial surface. CPS are usually complex, linear or branched, and of larger molecular weight than LPS. Their high immunogenicity and surface exposure had made them interesting targets for vaccination strategies. For instance, WO2016156338 discloses conjugates of synthetic oligosaccharides that are related to carbapenem-resistant K. pneumoniae CPS.

The variability, however, of Klebsiella CPS is high. Serologically more than 77 different CPS types, so-called K-types, K-serotypes or K-antigens have been identified, but there are at least 141 K-types. These additional K-types are identified based on the cps-locus or the K-locus and are called the KL series.

On the other hand, the variability of LPS is lower and the currently known so-called O-types, O-serotypes or O-antigens are limited to 11 major groups: O1, O2a, O2ac, O2afg, O2aeh (previously O9), O3 (includes sub-serotypes O3, O3a and O3b), O4, O5, O7, O8, and O12. Other than the above 11 additional O-types have been reported based on the O-locus known as the OL series. Though O-antigens are less immunogenic than K-antigens and are exposed to a lesser extent to the surface of the membrane, they have also been considered for vaccination strategies. In a recent survey a large collection of clinical isolates determined the relative prevalence of lipopolysaccharide (LPS) serotypes, particularly in multidrug-resistant isolates. The O1 antigen plays globally a major role as antigen in K. pneumoniae infections, in particular in Americas, Asia and Africa. WO2019106201 discloses conjugates of synthetic oligosaccharides related to K. pneumoniae serotype O1, O2, O2ac, and O8 O-polysaccharide and carbapenem-resistant K. pneumoniae ST258 O-polysaccharide.

In particular, WO2019106201 discloses an octasaccharide-carrier protein conjugate, namely compound 61* conjugated to CRM₁₉₇, which led to the production of IgG in immunization experiments with mice. The obtained sera recognized the corresponding O-antigen BSA conjugate in an ELISA.

However, to date, there are no approved vaccines available against K. pneumoniae, which demonstrates clearly the challenges associated with the development of such vaccines.

It remains difficult and unpredictable whether or which CPS or LPS may be appropriate candidates or model sequences for vaccines, and in particular it is unpredictable whether and which shorter oligosaccharides would be suitable for generating the desired immune response in vivo.

It has now been found that specific oligosaccharide-carrier protein conjugates show improved immunological properties over the state of the art.

### DESCRIPTION OF THE FIGURES

FIG. 1: Characterisation of C7-CRM₁₉₇* glycoconjugate in comparison to CRM₁₉₇ by HPLC-SEC.

FIG. 2: SDS-PAGE of C7-CRM₁₉₇* glycoconjugate in comparison to CRM₁₉₇ and Marker (protein size marker is GelCode™ Blue Safe Protein Stain (Thermo Scientific)).

FIG. 3: Shows a mouse immunogenicity test in BALB/c-mice (6 mice) with 5 μg Compar-CRM₁₉₇* antigen dose per mouse per immunization on day 0, 14 and 28; i.e. FIG. 3A shows the ELISA against corresponding BSA glycoconjugate; FIG. 3B shows ELISA against O1 LPS isolated from the PCM12 strain (Polish Collection of Microorganisms) using an LPS extraction kit (JH Science); sera were diluted as indicated in the graph.

FIG. 4: Shows a mouse immunogenicity test in C57BL/6 mice (6 mice) with 2.5 μg C7-CRM₁₉₇* antigen dose per mouse per immunization on day 0, 14, 28; i.e. FIG. 4 shows the ELISA against corresponding BSA glycoconjugate; sera were diluted as indicated in the graph.

FIG. 5: Shows a mouse immunogenicity test in C57BL/6 mice (6 mice) with 2.5 μg C7-CRM₁₉₇* antigen dose per mouse per immunization on day 0, 14, 28; i.e. FIG. 5 shows the ELISA against isolated LPS on day 35, pooled sera diluted 1:100. LPS was isolated from strains Friedländer (O1), NCTC 9148 (O2a) or PCM27 (Gal III) using an LPS extraction kit (JH Science).

FIG. 6: Shows rabbit immunogenicity results (ELISA against corresponding BSA conjugate). The bars represent pooled sera of 4 animals at the serum dilutions indicated in the graph. The rabbits were immunized with 2 μg C7-CRM₁₉₇* antigen dose per animal per immunization on days 0, 21 and 35 and serum samples were taken on days 0, 7, 28 and 42. “Blank” is secondary antibody (Goat anti-Rabbit IgG-HRP, SIGMA A4914, diluted 1:10,000) only.

FIG. 7: Shows ELISA-inferred binding of rabbit IgG to isolated LPS of an O1-expressing strain (PCM12). Data for 4 individual rabbits are shown (1:100 serum dilution); the bars represent mean values. The rabbits were immunized with 2 μg C7-CRM₁₉₇* antigen dose per animal per immunization on days 0, 21 and 35 and serum samples were taken on days 0, 7, 28 and 42. “Blank” is secondary antibody (Goat anti-Rabbit IgG-HRP, SIGMA A4914, diluted 1:10,000) only.

FIG. 8: Shows survival data of a challenge experiment in mice. CD-1 mice (10 per arm) received two intraperitoneal injections of 250 μL rabbit antisera generated with C7-CRM₁₉₇* (obtained by immunization with 2 μg C7-CRM₁₉₇* antigen dose per rabbit per immunization on days 0, 14 and 28, and collected on day 35) at −24 h and −1 h relative to infection or control antiserum generated with placebo (Aluminum hydroxide adjuvant (Brenntag) in buffer). At 0 h, the mice were infected intraperitoneally with a lethal dose of a Klebsiella pneumoniae O1-expressing strain PCM12 along with galactosamine treatment (20 mg/mouse intraperitoneally). The mice were observed for 24 h for survival. The survival curves are statistically significantly different with P=0.0045 (Log-rank (Mantel-Cox test)).

FIG. 9: Shows survival data of a challenge experiment in mice. C57BL/6 mice (8 per arm) were immunized with 2 μg C7-CRM₁₉₇* antigen dose per animal per immunization or placebo (adjuvant aluminum hydroxide in buffer) at −43 d, −27 d and −15 d relative to infection. At 0 h, the mice were infected intraperitoneally with a lethal dose of a Klebsiella pneumoniae O1-expressing strain PCM12 (Polish Collection of Microorganisms) in the presence of 5% mucin. The mice were observed for 24 h for survival. The survival curves are statistically significantly different with P=0.0007 (Log-rank (Mantel-Cox test)).

FIG. 10: amino acid sequence SEQ ID NO: 1 of CRM₁₉₇.

### DETAILED DESCRIPTION OF THE INVENTION

1) In a first embodiment, the present invention relates to an oligosaccharide-carrier protein conjugate of formula (I)

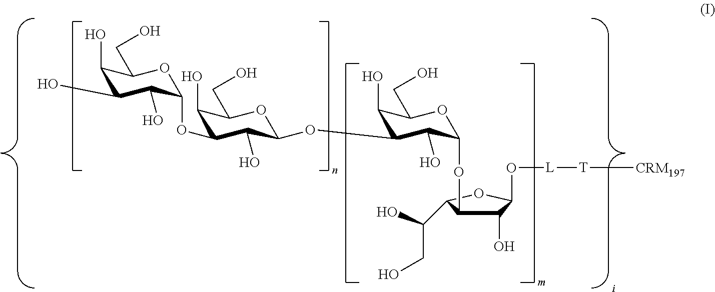

wherein
m is 4, 5 or 6;
n is 5, 6 or 7;
i is from 1 to 28; and
-L-T-represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, wherein the atoms of the backbone are selected from the group consisting of carbon, nitrogen, oxygen and sulphur;
   or a pharmaceutically acceptable salt thereof.

Definitions provided herein are intended to apply uniformly to the compounds of Formula (I), (II), (III) and (IV) as defined in any one of embodiments 1) to 57), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The oligosaccharide part (i.e. the epitope or antigen-part) of the compounds of Formula (I), (II), (III) and (IV) is composed of D-galacto-pyranosides and D-galacto-furanosides, respectively. The configuration at each anomeric center is either alpha or beta. The configuration at the anomeric centers may contribute to a mixture of anomers, whereby the anomers are synthesized in alpha or beta form, preferably as pure alpha or beta anomers. Mixtures of anomers may be separated in a manner known to a person skilled in the art.

The term “essentially”, for example when used in a term such as “essentially pure” is understood in the context of the present invention to mean especially that the respective oligosaccharide/oligosaccharide-linker compound/oligosaccharide-linker-spacer compound/glycoconjugate consists in an amount of at least 90, especially of at least 95, and notably of at least 99 percent by weight of the respective pure oligosaccharide/oligosaccharide-linker compound/oligosaccharide-linker-spacer compound/glycoconjugate.

Whenever a substituent is denoted as optional, it is understood that such substituent may be absent (i.e. the respective residue is unsubstituted with regard to such optional substituent), in which case all positions having a free valency (to which such optional substituent could have been attached to; such as for example in an aromatic ring the ring carbon atoms and/or the ring nitrogen atoms having a free valency) are substituted with hydrogen where appropriate. Likewise, in case the term “optionally” is used in the context of (ring) heteroatom(s), the term means that either the respective optional heteroatom(s), or the like, are absent (i.e. a certain moiety does not contain heteroatom(s)/is a carbocycle/or the like), or the respective optional heteroatom(s), or the like, are present as explicitly defined.

In the present application, the term “lysine residue” and “lysine site” are used synonymously.

The oligosaccharide of the present invention is composed of galactans, namely
beta-D-galactofuranose/β-D-Galf.

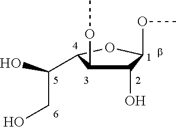

the dotted lines show the point of attachment, namely C1 and C3
alpha-D-galactopyranose/α-D-Galp:

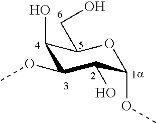

the dotted lines show the point of attachment, namely C1 and C3
beta-D-galactopyranose/β-D-Galp:

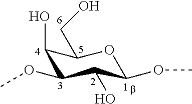

the dotted lines show the point of attachment, namely C1 and C3

The term “oligosaccharide-carrier protein conjugate” as used herein is taken synonymously to the term “glycoconjugate”.

“CRM₁₉₇” refers to Cross Reactive Material 197, which is a nontoxic mutant version of the diphtheria toxin, wherein the single amino acid exchange of a glycine (Gly, G) in position 52 to a glutamic acid (Glu, E) renders the protein non-toxic.

CRM₁₉₇ is produced by C. diphtheriae infected by the nontoxigenic phage β197tox created by nitrosoguanidine mutagenesis of the toxigenic corynephage beta (Uchida et al, J. Biol. Chem., 1973, Vol. 245, No. 11, pp. 3838-3844). The CRM₁₉₇ protein is a safe and effective T-cell dependent carrier for saccharides. CRM₁₉₇ is for instance described by Giannini et al. in Nucleic Acids Research, Vol 12, No. 10, 1984, pp. 4063-4069. Further details about CRM₁₉₇ and production thereof can be found e.g. in U.S. Pat. No. 5,614,382, which are incorporated herein by reference. CRM₁₉₇ may be produced in various expression systems, for instance in Corynebacterium diphtheriae, Escherichia coli or Pseudomonas fluorescens (Hickey et al, J. Pharm. Sci., 2018, 107, 1806-1819).

In the present invention, the term “CRM₁₉₇” encompasses a protein having at least 90%, 91%, 92%, 93%, 94%, 95%, 96%, 97%, 98%, 99%, 99.8% or 99.9% identity to amino acid sequence SEQ ID NO: 1 (preferably 90%, 91%, 92%, 93%, 94%, 95%, 96%, 97%, 98%, 99% or 99.9% identity to amino acid sequence SEQ ID NO: 1; and notably 95%, 96%, 97%, 98%, 99% or 99.9% identity to amino acid sequence SEQ ID NO: 1), which optionally comprises an additional methionine (Met, M) at the N-terminus, and/or optionally includes residues resulting from functionalizing CRM₁₉₇ at lysine sites, which residues may be in a capped (i.e. deactivated) form.

The phrase “residues resulting from functionalizing CRM₁₉₇ at lysine sites” means that CRM₁₉₇ is functionalized at lysine sites with functional groups suitable for forming a covalent bond to the linker and/or spacer part attached to the epitope, i.e. the oligosaccharide-linker part of the conjugate. Such lysine-functionalized CRM₁₉₇ is known to the skilled person. The functional groups are particularly suitable for linking thiols or for performing click-chemistry. For instance, such functional groups are groups containing a bromo-acetamide, a iodo-acetamide, a maleimide, an azido, or an alkyne group. This means that CRM₁₉₇ optionally includes lysine residues functionalized with a bromo-acetamide, a iodo-acetamide, a maleimide, an azido, or an alkyne group, (preferably a bromo-acetamide, a iodo-acetamide, a maleimide group), which groups may be in a capped form.

Preferred functionalized CRM₁₉₇ contains groups carrying bromo-acetamide, iodo-acetamide or maleimide groups, all of them being suitable for reaction with thiol-groups provided by the oligosaccharide/linker moiety. Unreacted functional groups at CRM₁₉₇ may subsequently be quenched with any pharmaceutically acceptable thiol, such as for instance L-cysteine or cysteamine (2-aminoethane-1-thiol) to give the “capped form”.

Preferred lysine-functionalized CRM₁₉₇ is selected from the group consisting of:

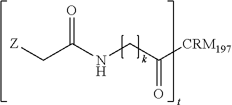

wherein Z is Br or I, k is 2 or 3, and t is from 1 to 28;

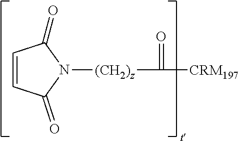

wherein z is 2 or 3, and t′ is from 1 to 28; and

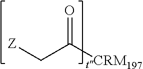

wherein Z is Br or I, and t″ is from 1 to 28.

In a preferred embodiment, CRM₁₉₇ is not functionalized in the above-described way. This means that there is no “pre-functionalization”, but rather, the “natural” lysine residues are used for directly attaching the oligosaccharide/linker/spacer part thereto.

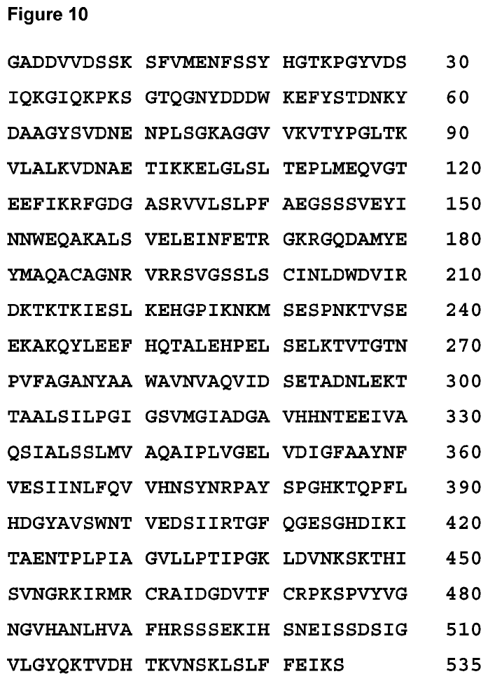

The amino acid sequence of CRM₁₉₇ is known to the skilled person, and is outlined in FIG. 10 as SEQ ID NO:1.

The use of CRM₁₉₇ for the synthesis of saccharide conjugates and preferred conjugation sites on CRM₁₉₇ has been reported (e.g. Möginger et al., Sci. Rep. 6, 20488; doi:10.1038/srep20488 (2016)), which is incorporated herein by reference.

The phrase “-L-T-represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, wherein the atoms of the backbone are selected from the group consisting of carbon, nitrogen, oxygen and sulphur” means that the backbone may be saturated, unsaturated, unsubstituted or substituted with one or more (especially 1, 2, 3 or 4) substituents independently selected from oxo, (C1-4)alkyl, fluoro, and (C1-2)alkoxy (especially oxo), and optionally a part of a ring structure may be part of the backbone. The ring structure may be a saturated, unsaturated or aromatic 3- to 8-membered ring including condensed ring systems of 2 to 4 rings, wherein the ring atoms are selected from carbon, nitrogen, oxygen and sulphur (especially from carbon and nitrogen), and the ring is unsubstituted or substituted with one or more (especially 1, 2, 3 or 4) substituents independently selected from oxo, (C1-4)alkyl, halogen, and (C1-2)alkoxy (especially oxo).

For avoidance of any doubt, the count of 5 to 25 atoms relates to the count of atoms of the backbone, not of the bridge.

Examples for the optional ring structures that may be part of the backbone are pyrrolidine-2,5-dione, cyclobut-3-ene-1,2-dione, triazole, isoindolin-1-one, 8,9-dihydro-1H-dibenzo[b,f][1,2,3]triazolo[4,5-d]azocine, cyclohexane, and benzene as follows:

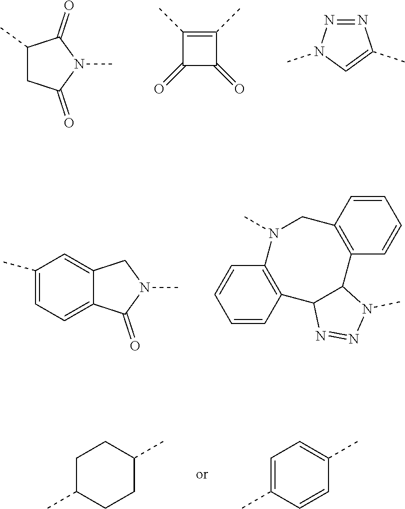

The introduction of these rings or ring systems is known to the skilled person in the field of linker chemistry.

The phrase “the backbone may be unsaturated” means that the backbone chain may contain one or more double bonds, which may or may not be part of a ring system.

For instance, the atom counting in a bridge having a saturated backbone with 3 oxo-substitutions and which backbone is part of a ring system is as follows:

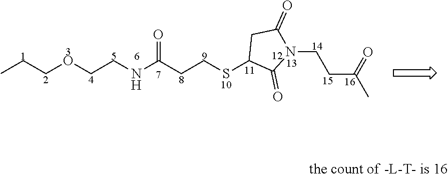 [SMILES: CCCOCCNC(=O)CCSC1CC(=O)N(CCC(C)=O)C1=O]

Hence, the count of the atoms forming the backbone starts with the first atom after the oxygen at C1 and ends with the last atom attached to a lysine nitrogen of CRM₁₉₇.

An oxygen atom in a saturated chain is preferably separated from another oxygen atom by one or more (especially 2, 3, 4 or 5, and notably 2) carbon atoms.

A sulphur atom in a saturated chain is preferably separated from another sulphur atom by one or more (especially 1, 2, 3, 4 or 5) carbon atoms.

The term “halogen” means fluorine, chlorine, or bromine, preferably fluorine or chlorine, more preferably fluorine.

The term “oxo” relates to the functional group ═O, i.e. a substituent oxygen atom connected to another atom (preferably a carbon atom) by a double bond.

The term “alkyl”, used alone or in combination, means a straight or branched saturated hydrocarbon chain containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, n-propyl, iso-propyl, n-butyl, iso-butyl, sec.-butyl and tert.-butyl. Examples of (C₁₋₂)alkyl groups are methyl and ethyl.

The term “alkoxy”, used alone or in combination, refers to an alkyl-O— group wherein the alkyl group is as defined before. The term “(C_{x-y})alkoxy” (x and y each being an integer) refers to an alkoxy group as defined before containing x to y carbon atoms. For example a (C₁₋₂)alkoxy group means a group of the formula (C₁₋₂)alkyl-O— in which the term “(C₁₋₂)alkyl” has the previously given significance. Examples of (C₁₋₂)alkoxy groups are methoxy and ethoxy.

2) A further embodiment relates to the oligosaccharide-carrier protein conjugate according to embodiment 1), or a pharmaceutically acceptable salt thereof, wherein m is 4 or 5.

3) A further embodiment relates to the oligosaccharide-carrier protein conjugate according to embodiment 1), or a pharmaceutically acceptable salt thereof, wherein m is 4.

4) A further embodiment relates to the oligosaccharide-carrier protein conjugate according to any one of embodiments 1), 2), or 3), or a pharmaceutically acceptable salt thereof, wherein n is 6 or 7.

5) A further embodiment relates to the oligosaccharide-carrier protein conjugate according to any one of embodiments 1), 2), or 3), or a pharmaceutically acceptable salt thereof, wherein n is 6.

6) A further embodiment relates to the oligosaccharide-carrier protein conjugate according to embodiment 1), or a pharmaceutically acceptable salt thereof, wherein m is 4 and n is 6.

7) A further embodiment relates to the oligosaccharide-carrier protein conjugate according to any one of embodiments 1), 2), 3), 4), 5) or 6), or a pharmaceutically acceptable salt thereof, wherein the bridge does not contain an aromatic or heteroaromatic ring.

8) A further embodiment relates to the oligosaccharide-carrier protein conjugate according to any one of embodiments 1), 2), 3), 4), 5) or 6), or a pharmaceutically accepable salt thereof, wherein
-L-T-represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, bearing at most one double bond,
   wherein the atoms of the backbone are selected from the group consisting of carbon, nitrogen, oxygen and sulphur, and
   wherein the backbone may be substituted with one or more (especially 1, 2, 3 or 4) substituents independently selected from oxo, (C₁₋₄)alkyl, fluoro, and (C₁₋₂)alkoxy (especially oxo), and
   wherein a part of the backbone optionally may be part of a 4-, 5- or 6-membered ring selected from:

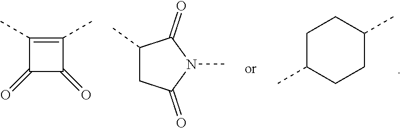

Thereby, the “at most one double bond” is preferably the double bond of the cyclobut-3-ene-1,2-dione ring.

9) A further embodiment relates to the oligosaccharide-carrier protein conjugate according to any one of embodiments 1), 2), 3), 4), 5) or 6), or a pharmaceutically accepable salt thereof, wherein
-L-T-represents a linker L and a spacer T which together form a bridge which consists of a backbone which is a saturated chain counting from 5 to 25 atoms selected from the group consisting of carbon, nitrogen, oxygen and sulphur (especially carbon, nitrogen and oxygen), which chain may be unsubstituted or substituted with one or more (especially 1, 2, 3 or 4) substituents independently selected from oxo, (C₁₋₄)alkyl, fluoro and (C₁-2)alkoxy (especially oxo).

This means that the bridge consists of a saturated chain counting from 5 to 25 atoms selected from the group consisting of carbon, nitrogen, oxygen and sulphur (especially carbon, nitrogen and oxygen), which chain may be unsubstituted or substituted with one or more (especially 1, 2, 3 or 4) substituents independently selected from oxo, (C₁₋₄)alkyl, fluoro and (C₁₋₂)alkoxy (especially oxo).

For the avoidance of any doubt, in this embodiment, the bridge does not contain a ring structure.

10) A further embodiment relates to the oligosaccharide-carrier protein conjugate according to any one of embodiments 1), 2), 3), 4), 5) or 6), or a pharmaceutically accepable salt thereof, wherein
-L-T-represents a linker L and a spacer T which together form a bridge which consists of a backbone which is a saturated chain counting from 5 to 25 atoms selected from the group consisting of carbon, nitrogen and oxygen (especially carbon and nitrogen), which chain may be unsubstituted or substituted with one or more (especially 1, 2, 3 or 4) substituents independently selected from oxo, (C₁₋₄)alkyl, fluoro, and (C₁₋₂)alkoxy (especially oxo).

This means that the bridge consists of a saturated chain counting from 5 to 25 atoms selected from the group consisting of carbon, nitrogen and oxygen (especially carbon and nitrogen), which chain may be unsubstituted or substituted with one or more (especially 1, 2, 3 or 4) substituents independently selected from oxo, (C₁₋₄)alkyl, fluoro, and (C₁₋₂)alkoxy (especially oxo).

For the avoidance of any doubt, in this embodiment, the bridge does not contain a ring structure.

11) A further embodiment relates to the oligosaccharide-carrier protein conjugate according to any one of embodiments 1), 2), 3), 4), 5), 6), 7), 8), 9) or 10), or a pharmaceutically accepable salt thereof, wherein the backbone of the bridge has a length of 8 to 20, preferably 8 to 16, atoms covalently linked together that forms the shortest distance between the oxygen at C1 of the reducing end of the oligosaccharide and the nitrogen of the amino group of a lysine residue at the carrier protein CRM₁₉₇.

12) A further embodiment relates to the oligosaccharide-carrier protein conjugate according to any one of embodiments 1), 2), 3), 4), 5), 6), or 11), or a pharmaceutically accepable salt thereof, wherein
L represents
   *—(C₂₋₁₀)alkylene-NH—;
   *—(CH₂CH₂O)ₐ—CH₂CH₂NH—, wherein a is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{d}—NH—, wherein c and d are independently from each other from 2 to 6;
   *—(CH₂)ₑNHC(O)NH(CH₂)ₕ—NH—, wherein e and h are independently from each other from 2 to 6;
   *—(C₁₋₁₀)alkylene-C(O)—NH—(C₂₋₁₀)alkylene-NH—; or
   *—(C₂₋₁₀)alkylene-O—NH—;
T represents
   —C(O)—(C₀₋₁₀)alkylene-C(O)—;
   —C(O)—CH₂CH₂—(OCH₂CH₂)ᵣC(O)—, wherein r is from 1 to 5;
   —C(O)—CH₂(CH₂)_{f}—(SCH₂(CH₂)_{f′})_{f″}—C(O)—, wherein f is 0 or 1, f′ is 0 or 1, and f″ is 1, 2, or 3;

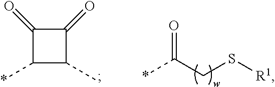

wherein w is 1 or 2; or

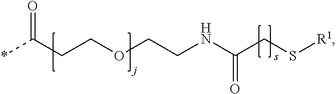

wherein j is from 1 to 4, and s is 1 or 2; or
L-T represents
   *—(C₂₋₁₀)alkylene-S—R¹; and
R¹ represents

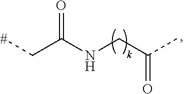

wherein k is 2 or 3;

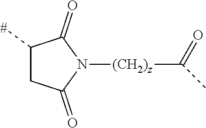

wherein z is 2 or 3; or

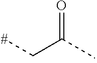

The “*” appointed in the linker L means that at this location, the linker is attached to the oligosaccharide.

The “*” appointed in the spacer T means that at this location, the spacer is attached to the linker L.

The “#” appointed in R¹ means that at this location, R¹ is attached to the sulphur.

The term “—(C_{x-y})alkylene-” (x and y each being an integer), used alone or in combination, refers to a bivalently bound saturated straight or branched hydrocarbon chain containing x to y carbon atoms. For example a (C₂₋₁₀)alkylene group contains from two to ten carbon atoms, and a (C₀₋₁₀)alkylene group is either a bond (i.e. absent, C being zero) or an alkylene group from one to ten carbon atoms. Straight —(C_{x-y})alkylene-, i.e. —(CH₂)_{x-y}— is preferred. Representative examples of (C₂₋₁₀)alkylene groups are ethylene, propylene, butylene, pentylene, hexylene, heptylene, octylene, nonylene and decylene (especially 1,2-ethylene, 1,3-propylene, 1,4-butylene, 1,5-pentylene, 1,6-hexylene, 1,7-heptylene, 1,8-octylene, 1,9-nonylene and 1,10-decylene).

The term “(C_{x-y})fluoroalkylene” (x and y each being an integer), used alone or in combination, refers to a bivalently bound saturated straight or branched chain hydrocarbon group containing x to y carbon atoms in which one or more (and possibly all) hydrogen atoms have been replaced with fluorine. Straight —(C_{x-y})fluoroalkylene- is preferred.

For the avoidance of any doubt, in the present embodiments, the length of the backbone of -L-T- is 5 to 25 atoms, 8 to 20 atoms, or 8 to 16 atoms. This means that the linker L and the spacer T, including R¹ where applicable, together form a bridge having a backbone with a length of 5 to 25 (8 to 20, or 8 to 16) atoms covalently linked together that forms the shortest distance between the oxygen at C1 of the reducing end of the oligosaccharide and the nitrogen of the amino group of a lysine residue at the carrier protein CRM₁₉₇.

13) A further embodiment relates to the oligosaccharide-carrier protein conjugate according to embodiment 12), or a pharmaceutically accepable salt thereof, wherein
L represents
   *—(CH₂)ₗ—NH—; wherein l is from 2 to 10;
   *—(CH₂CH₂O)ₐ—CH₂CH₂NH—, wherein a is 1, 2 or 3;
   *—CH₂CH₂S—CH₂CH₂NH—;
   *—(C₂₋₁₀)fluoroalkylene-NH— with fluoroalkylene being a saturated straight chain;
   *—(CH₂)_{c}NHC(O)(CH₂)_{d}—NH—, wherein c and d are independently from each other from 2 to 6;
   *—(CH₂)ₑNHC(O)NH(CH₂)ₕ—NH—, wherein e and h are independently from each other from 2 to 6;
   *—(CH₂)ᵤ—C(O)—NH—(CH₂)_{u′}—NH—; wherein u is from 1 to 10 and u′ is from 2 to 10; or
   *—(CH₂)_{g}—O—NH—, wherein g is from 2 to 10; or
L-T represents
   *—(CH₂)_{q}S—R¹, wherein q is from 2 to 10.

14) A further embodiment relates to the oligosaccharide-carrier protein conjugate according to embodiment 12), or a pharmaceutically accepable salt thereof, wherein
L represents
   *—(CH₂)ₗ—NH—; wherein l is from 2 to 10;
   *—(CH₂CH₂O)ₐ—CH₂CH₂NH—, wherein a is 1, 2 or 3;
   *—(C₂₋₁₀)fluoroalkylene-NH— with fluoroalkylene being a saturated straight chain;
   *—(CH₂)ᵤ—C(O)—NH—(CH₂)_{u′}—NH—; wherein u is from 1 to 10 and u′ is from 2 to 10; or
   *—(CH₂)_{g}—O—NH—, wherein g is from 2 to 10; or
L-T represents
   *—(CH₂)_{q}S—R¹, wherein q is from 2 to 10.

15) A further embodiment relates to the oligosaccharide-carrier protein conjugate according to embodiment 12), or a pharmaceutically accepable salt thereof, wherein
L represents
   *—(CH₂)ₗ—NH—; wherein l is from 2 to 10, preferably from 2 to 6;
   *—(CH₂CH₂O)ₐ—CH₂CH₂NH—, wherein a is 1, 2 or 3, preferably 1 or 2;
   *—(CH₂)ᵤ—C(O)—NH—(CH₂)_{u′}—NH—; wherein u is from 1 to 10, preferably from 1 to 6 and u′ is from 2 to 10, preferably from 2 to 6; or
   *—(CH₂)_{g}—O—NH—, wherein g is from 2 to 10, preferably from 2 to 6; or
L-T represents
   *—(CH₂)_{q}S—R¹, wherein q is from 2 to 10, preferably from 2 to 6.

16) A further embodiment relates to the oligosaccharide-carrier protein conjugate according to embodiment 12), or a pharmaceutically accepable salt thereof, wherein
L represents
   *—(CH₂)ₗ—NH—; wherein l is from 2 to 10, preferably from 2 to 6;
   *—(CH₂CH₂O)ₐ—CH₂CH₂NH—, wherein a is 1, 2 or 3, preferably 1 or 2; or
   *—(CH₂)_{g}—O—NH—, wherein g is from 2 to 10, preferably from 2 to 6.

17) A further embodiment relates to the oligosaccharide-carrier protein conjugate according to embodiment 12), or a pharmaceutically accepable salt thereof, wherein
L represents
   *—(CH₂)ₗ—NH—; wherein l is from 2 to 10, preferably from 2 to 6; or
   —(CH₂CH₂O)ₐ—CH₂CH₂NH—, wherein a is 1, 2 or 3, preferably 1 or 2;

18) A further embodiment relates to the oligosaccharide-carrier protein conjugate according to embodiment 12), or a pharmaceutically accepable salt thereof, wherein L represents *—(CH₂)ₗ—NH—; wherein l is from 2 to 10, preferably from 2 to 6.

19) A further embodiment relates to the oligosaccharide-carrier protein conjugate according to embodiment 12), or a pharmaceutically accepable salt thereof, wherein
L represents *—(CH₂)₂—NH—, *—(CH₂)₃—NH—, *—(CH₂)₄—NH—, *—(CH₂)₅—NH—, or *—(CH₂)₆—NH—.

20) A further embodiment relates to the oligosaccharide-carrier protein conjugate according to embodiment 12), or a pharmaceutically accepable salt thereof, wherein L represents *—(CH₂)₅—NH—.

21) A further embodiment relates to the oligosaccharide-carrier protein conjugate according to embodiment 12), or a pharmaceutically accepable salt thereof, wherein L represents *—(CH₂CH₂O)ₐ—CH₂CH₂NH—, wherein a is 1 or 2; preferably, a is 1.

22) A further embodiment relates to the oligosaccharide-carrier protein conjugate according to any one of embodiments 12) to 21), or a pharmaceutically accepable salt thereof, wherein
T represents
   —C(O)—(CH₂)ₚ—C(O)—, wherein p is from 0 to 10;
   —C(O)—CH₂CH₂—(OCH₂CH₂)ᵣ—C(O)—, wherein r is from 1 to 5;
   —C(O)—CH₂ (CH₂)_{f}—(SCH₂ (CH₂)_{f′})_{f″}—C(O)—, wherein f is 0 or 1, f′ is 0 or 1, and f″ is 1, 2, or 3

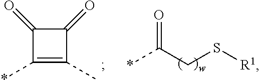

wherein w is 1 or 2; or

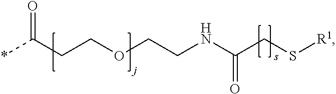

wherein j is from 1 to 4, and s is 1 or 2.

23) A further embodiment relates to the oligosaccharide-carrier protein conjugate according to any one of embodiments 12) to 21), or a pharmaceutically accepable salt thereof, wherein
T represents
   —C(O)—(CH₂)ₚ—C(O)—, wherein p is from 0 to 10, preferably from 0 to 6;
   —C(O)—CH₂CH₂—(OCH₂CH₂)ᵣC(O)—, wherein r is from 1 to 5, preferably from 1 to 3, more preferably 1;
   —C(O)—CH₂ (CH₂)_{f}—(SCH₂ (CH₂)_{f′})_{f″}—C(O)—, wherein f is 0 or 1, f′ is 0 or 1, and f″ is 1, 2, or 3, preferably 1; or

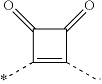

24) A further embodiment relates to the oligosaccharide-carrier protein conjugate according to any one of embodiments 12) to 21), or a pharmaceutically accepable salt thereof, wherein
T represents
   —C(O)—(CH₂)ₚ—C(O)—, wherein p is from 0 to 6;
   —C(O)—CH₂CH₂—(OCH₂CH₂)ᵣ—C(O)—, wherein r is 1 or 2; or
   —C(O)—CH₂ (CH₂)_{f}—(SCH₂ (CH₂)_{f′})_{f″}—C(O)—, wherein f is 0 or 1, f′ is 0 or 1, and f″ is 1, preferably wherein f and f′ are 0 and f″ is 1.

25) A further embodiment relates to the oligosaccharide-carrier protein conjugate according to any one of embodiments 12) to 21), or a pharmaceutically accepable salt thereof, wherein
T represents
   —C(O)—(CH₂)ₚ—C(O)—, wherein p is 0, 1, 2, 3, 4, 5, or 6, preferably 4; or
   —C(O)—CH₂CH₂—(OCH₂CH₂)ᵣ—C(O)—, wherein r is 1 or 2.

26) A further embodiment relates to the oligosaccharide-carrier protein conjugate according to any one of embodiments 12) to 21), or a pharmaceutically accepable salt thereof, wherein
T represents —C(O)—(CH₂)₄—C(O)—.

27) A further embodiment relates to the oligosaccharide-carrier protein conjugate according to any one of embodiments 12) to 21), or a pharmaceutically accepable salt thereof, wherein
T represents

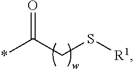

wherein w is 1 or 2; or

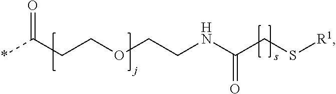

wherein j is from 1 to 4, preferably 1, and s is 1 or 2.

28) A further embodiment relates to the oligosaccharide-carrier protein conjugate according to any one of embodiments 12) to 21), or a pharmaceutically accepable salt thereof, wherein
T represents

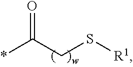

wherein w is 1 or 2.

29) A further embodiment relates to the oligosaccharide-carrier protein conjugate according to any one of embodiments 12), 13), 14), 15), 22), 27), and 28), or a pharmaceutically accepable salt thereof, wherein
R¹ represents

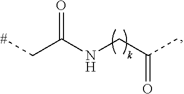

wherein k is 2 or 3;

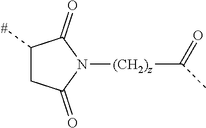

wherein z is 2 or 3; or

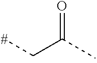

Preferably, R¹ represents:

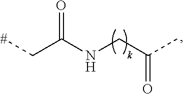

wherein k is 2 or 3; or

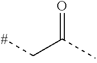

30) A further embodiment relates to the oligosaccharide-carrier protein conjugate according to any one of embodiments 1), 2), 3), 4), 5) or 6), or a pharmaceutically accepable salt thereof, wherein
L represents
   —(CH₂)₂—NH—, *—(CH₂)₃—NH—, *—(CH₂)₄—NH—, or *—(CH₂)₅—NH—; and
T represents
   —C(O)—(CH₂)₄—C(O)—.

31) A further embodiment relates to the oligosaccharide-carrier protein conjugate according to any one of embodiments 1), 2), 3), 4), 5) or 6), or a pharmaceutically accepable salt thereof, wherein
L represents *—(CH₂)₅—NH— and T represents —C(O)—(CH₂)₄—C(O)—.

32) A preferred embodiment is the oligosaccharide-carrier protein conjugate with the following formula:

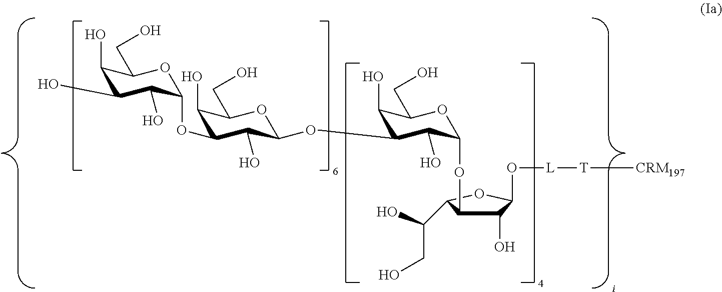

wherein L, T and i are as defined in any one of embodiments 1), or 7) to 31), or a pharmaceutically accepable salt thereof.

33) A further embodiment relates to the oligosaccharide-carrier protein conjugate according to any one of embodiments 1) to 32), or a pharmaceutically accepable salt thereof, wherein i is from 1 to 28, 1 to 25, 1 to 23; 1 to 20, 1 to 18, 3 to 25, 3 to 23, 3 to 20, 3 to 18, 5 to 23, 5 to 20, 5 to 18, 6 to 23, 6 to 20, 6 to 18, 6 to 15.

The variable i describes the loading of epitopes/oligosaccharides on the CRM₁₉₇ protein carrier and is an integer in respect of one single molecule. However, when considering the glycoconjugate as a product of more than one single molecule, it has to be noted that the loading can be described as a statistical distribution, i.e. essentially a Gaussian distribution. The chemical process of producing the product results in a mixture of molecules with such statistical distribution of the loading, and the loading is then provided as the mean of the statistical distribution, in particular the Gaussian distribution.

It is to be understood that for i≥2, m and/or n of the two or more oligosaccharides, that are attached via -L-T- to CRM₁₉₇, may be the same or different. Preferably, all i oligosaccharides are represented by the same combination of m and n (i.e. have identical structures) or all i oligosaccharides are represented by a first combination of m and n or a second combination of m and n (i.e. have one or another structure); most preferably all i oligosaccharides are represented by the same combination of m and n. The linker-spacer unit-L-T- is identical for the i oligosaccharides of a specific oligosaccharide-carrier protein conjugate.

In other words, preferred oligosaccharide-carrier protein conjugates are those that have uniform oligosaccharide/linker/spacer residues, i.e. which bear only one specific type of oligosaccharide/linker/spacer residue attached to the carrier CRM₁₉₇.

34) A further embodiment relates to the oligosaccharide-carrier protein conjugate according to any one of embodiments 1) to 32), or a pharmaceutically accepable salt thereof, wherein i is from 6 to 15.

35) A preferred embodiment relates to the oligosaccharide-carrier protein conjugate, wherein the oligosaccharide-carrier protein conjugate has the structure of formula (Ib):

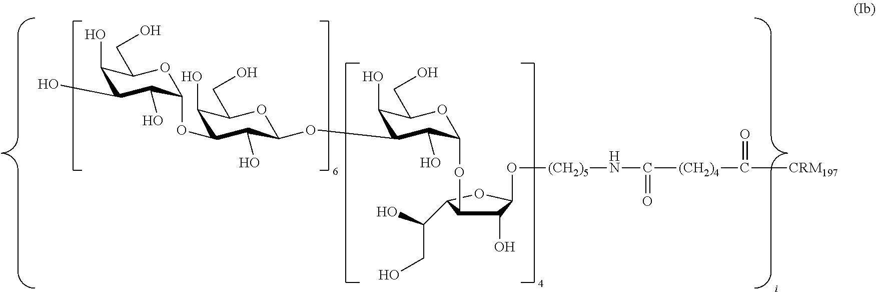

wherein i is from 1 to 28, or a pharmaceutically acceptable salt thereof.

For the avoidance of any doubt, the oligosaccharide-carrier protein conjugate of formula (Ib) according to this embodiment can also be schematically drawn as follows:

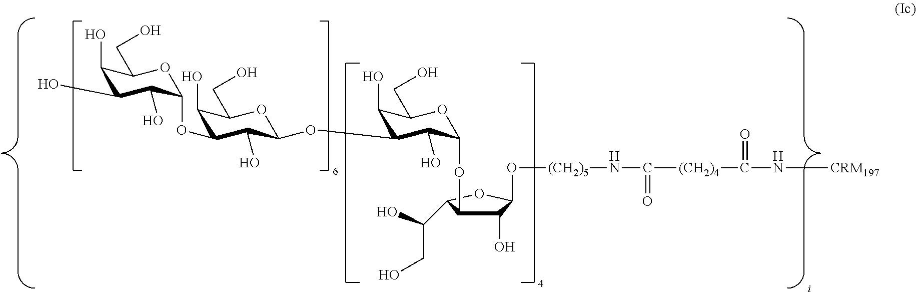

wherein i is from 1 to 28, or a pharmaceutically acceptable salt thereof. CRM₁₉₇′ means CRM₁₉₇ as defined herein, with the only difference in that in formula (Ic), the amino-group of the lysine residue is specifically shown as the attachment position of the linker/spacer in part -L-T-.

36) A further preferred embodiment relates to the oligosaccharide-carrier protein conjugate, wherein the oligosaccharide-carrier protein conjugate has the structure of formula (Ib):

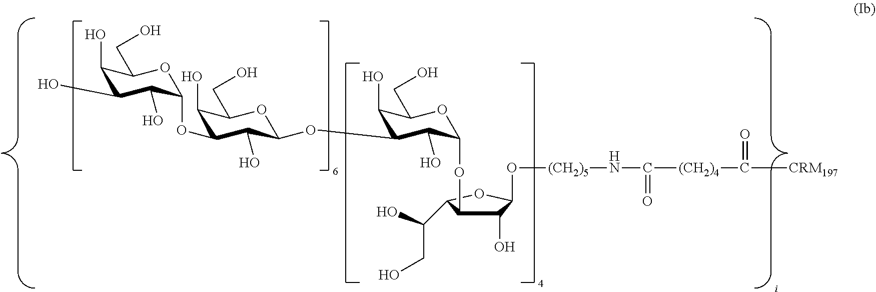

wherein i is from 6 to 15, or a pharmaceutically acceptable salt thereof.

For the avoidance of any doubt, the oligosaccharide-carrier protein conjugate of formula (Ib) according to this embodiment can also be schematically drawn as follows:

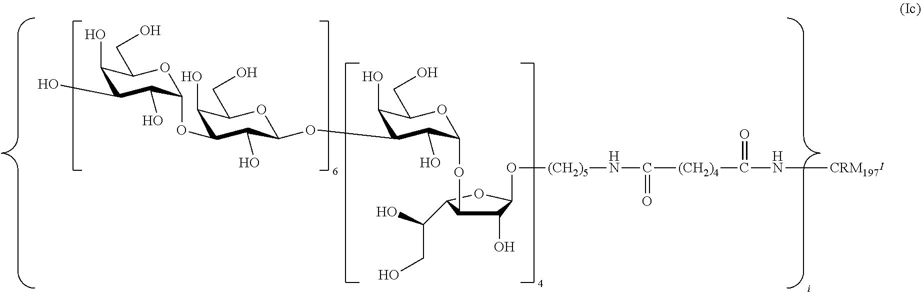

wherein i is from 6 to 15, or a pharmaceutically acceptable salt thereof.

The invention, thus, relates to compounds of the Formula (I) as defined in embodiment 1), and to such compounds further limited by the characteristics of any one of embodiments 2) to 36), under consideration of their respective dependencies; to pharmaceutically acceptable salts thereof; and to the use of such compounds as further described below. In particular, compounds of Formula (Ia), (Ib) and (Ic) are sub-forms of Formula (I). For avoidance of doubt, especially the following embodiments relating to the compounds of Formula (I) are thus possible and intended and herewith specifically disclosed in individualized form:

1, 2 + 1, 3 + 1, 4 + 1, 4 + 2 + 1, 4 + 3 + 1, 5 + 1, 5 + 2 + 1, 5 + 3 + 1, 6 + 1, 7 + 1, 7 + 2 + 1, 7 + 3 + 1, 7 + 4 + 1, 7 + 4 + 2 + 1, 7 + 4 + 3 + 1,
7 + 5 + 1, 7 + 5 + 2 + 1, 7 + 5 + 3 + 1, 7 + 6 + 1, 8 + 1, 8 + 2 + 1, 8 + 3 + 1, 8 + 4 + 1, 8 + 4 + 2 + 1, 8 + 4 + 3 + 1, 8 + 5 + 1, 8 + 5 + 2 + 1,
8 + 5 + 3 + 1, 8 + 6 + 1, 9 + 1, 9 + 2 + 1, 9 + 3 + 1, 9 + 4 + 1, 9 + 4 + 2 + 1, 9 + 4 + 3 + 1, 9 + 5 + 1, 9 + 5 + 2 + 1, 9 + 5 + 3 + 1, 9 + 6 + 1, 10 + 1,
10 + 2 + 1, 10 + 3 + 1, 10 + 4 + 1, 10 + 4 + 2 + 1, 10 + 4 + 3 + 1, 10 + 5 + 1, 10 + 5 + 2 + 1, 10 + 5 + 3 + 1, 10 + 6 + 1, 11 + 1, 11 + 2 + 1,
11 + 3 + 1, 11 + 4 + 1, 11 + 4 + 2 + 1, 11 + 4 + 3 + 1, 11 + 5 + 1, 11 + 5 + 2 + 1, 11 + 5 + 3 + 1, 11 + 6 + 1, 11 + 7 + 1, 11 + 7 + 2 + 1,
11 + 7 + 3 + 1, 11 + 7 + 4 + 1, 11 + 7 + 4 + 2 + 1, 11 + 7 + 4 + 3 + 1, 11 + 7 + 5 + 1, 11 + 7 + 5 + 2 + 1, 11 + 7 + 5 + 3 + 1, 11 + 7 + 6 + 1, 11 + 8 + 1,
11 + 8 + 2 + 1, 11 + 8 + 3 + 1, 11 + 8 + 4 + 1, 11 + 8 + 4 + 2 + 1, 11 + 8 + 4 + 3 + 1, 11 + 8 + 5 + 1, 11 + 8 + 5 + 2 + 1, 11 + 8 + 5 + 3 + 1,
11 + 8 + 6 + 1, 11 + 9 + 1, 11 + 9 + 2 + 1, 11 + 9 + 3 + 1, 11 + 9 + 4 + 1, 11 + 9 + 4 + 2 + 1, 11 + 9 + 4 + 3 + 1, 11 + 9 + 5 + 1, 11 + 9 + 5 + 2 + 1,
11 + 9 + 5 + 3 + 1, 11 + 9 + 6 + 1, 11 + 10 + 1, 11 + 10 + 2 + 1, 11 + 10 + 3 + 1, 11 + 10 + 4 + 1, 11 + 10 + 4 + 2 + 1, 11 + 10 + 4 + 3 + 1,
11 + 10 + 5 + 1, 11 + 10 + 5 + 2 + 1, 11 + 10 + 5 + 3 + 1, 11 + 10 + 6 + 1, 12 + 1, 12 + 2 + 1, 12 + 3 + 1, 12 + 4 + 1, 12 + 4 + 2 + 1,
12 + 4 + 3 + 1, 12 + 5 + 1, 12 + 5 + 2 + 1, 12 + 5 + 3 + 1, 12 + 6 + 1, 12 + 11 + 1, 12 + 11 + 2 + 1, 12 + 11 + 3 + 1, 12 + 11 + 4 + 1,
12 + 11 + 4 + 2 + 1, 12 + 11 + 4 + 3 + 1, 12 + 11 + 5 + 1, 12 + 11 + 5 + 2 + 1, 12 + 11 + 5 + 3 + 1, 12 + 11 + 6 + 1, 13 + 12 + 1, 13 + 12 + 2 + 1,
13 + 12 + 3 + 1, 13 + 12 + 4 + 1, 13 + 12 + 4 + 2 + 1, 13 + 12 + 4 + 3 + 1, 13 + 12 + 5 + 1, 13 + 12 + 5 + 2 + 1, 13 + 12 + 5 + 3 + 1,
13 + 12 + 6 + 1, 13 + 12 + 11 + 1, 13 + 12 + 11 + 2 + 1, 13 + 12 + 11 + 3 + 1, 13 + 12 + 11 + 4 + 1, 13 + 12 + 11 + 4 + 2 + 1,
13 + 12 + 11 + 4 + 3 + 1, 13 + 12 + 11 + 5 + 1, 13 + 12 + 11 + 5 + 2 + 1, 13 + 12 + 11 + 5 + 3 + 1, 13 + 12 + 11 + 6 + 1, 14 + 12 + 1,
14 + 12 + 2 + 1, 14 + 12 + 3 + 1, 14 + 12 + 4 + 1, 14 + 12 + 4 + 2 + 1, 14 + 12 + 4 + 3 + 1, 14 + 12 + 5 + 1, 14 + 12 + 5 + 2 + 1,
14 + 12 + 5 + 3 + 1, 14 + 12 + 6 + 1, 14 + 12 + 11 + 1, 14 + 12 + 11 + 2 + 1, 14 + 12 + 11 + 3 + 1, 14 + 12 + 11 + 4 + 1, 14 + 12 + 11 + 4 + 2 + 1,
14 + 12 + 11 + 4 + 3 + 1, 14 + 12 + 11 + 5 + 1, 14 + 12 + 11 + 5 + 2 + 1, 14 + 12 + 11 + 5 + 3 + 1, 14 + 12 + 11 + 6 + 1, 15 + 12 + 1,
15 + 12 + 2 + 1, 15 + 12 + 3 + 1, 15 + 12 + 4 + 1, 15 + 12 + 4 + 2 + 1, 15 + 12 + 4 + 3 + 1, 15 + 12 + 5 + 1, 15 + 12 + 5 + 2 + 1,
15 + 12 + 5 + 3 + 1, 15 + 12 + 6 + 1, 15 + 12 + 11 + 1, 15 + 12 + 11 + 2 + 1, 15 + 12 + 11 + 3 + 1, 15 + 12 + 11 + 4 + 1, 15 + 12 + 11 + 4 + 2 + 1,
15 + 12 + 11 + 4 + 3 + 1, 15 + 12 + 11 + 5 + 1, 15 + 12 + 11 + 5 + 2 + 1, 15 + 12 + 11 + 5 + 3 + 1, 15 + 12 + 11 + 6 + 1, 16 + 12 + 1,
16 + 12 + 2 + 1, 16 + 12 + 3 + 1, 16 + 12 + 4 + 1, 16 + 12 + 4 + 2 + 1, 16 + 12 + 4 + 3 + 1, 16 + 12 + 5 + 1, 16 + 12 + 5 + 2 + 1,
16 + 12 + 5 + 3 + 1, 16 + 12 + 6 + 1, 16 + 12 + 11 + 1, 16 + 12 + 11 + 2 + 1, 16 + 12 + 11 + 3 + 1, 16 + 12 + 11 + 4 + 1, 16 + 12 + 11 + 4 + 2 + 1,
16 + 12 + 11 + 4 + 3 + 1, 16 + 12 + 11 + 5 + 1, 16 + 12 + 11 + 5 + 2 + 1, 16 + 12 + 11 + 5 + 3 + 1, 16 + 12 + 11 + 6 + 1, 17 + 12 + 1,
17 + 12 + 2 + 1, 17 + 12 + 3 + 1, 17 + 12 + 4 + 1, 17 + 12 + 4 + 2 + 1, 17 + 12 + 4 + 3 + 1, 17 + 12 + 5 + 1, 17 + 12 + 5 + 2 + 1,
17 + 12 + 5 + 3 + 1, 17 + 12 + 6 + 1, 17 + 12 + 11 + 1, 17 + 12 + 11 + 2 + 1, 17 + 12 + 11 + 3 + 1, 17 + 12 + 11 + 4 + 1, 17 + 12 + 11 + 4 + 2 + 1,
17 + 12 + 11 + 4 + 3 + 1, 17 + 12 + 11 + 5 + 1, 17 + 12 + 11 + 5 + 2 + 1, 17 + 12 + 11 + 5 + 3 + 1, 17 + 12 + 11 + 6 + 1, 18 + 12 + 1,
18 + 12 + 2 + 1, 18 + 12 + 3 + 1, 18 + 12 + 4 + 1, 18 + 12 + 4 + 2 + 1, 18 + 12 + 4 + 3 + 1, 18 + 12 + 5 + 1, 18 + 12 + 5 + 2 + 1,
18 + 12 + 5 + 3 + 1, 18 + 12 + 6 + 1, 18 + 12 + 11 + 1, 18 + 12 + 11 + 2 + 1, 18 + 12 + 11 + 3 + 1, 18 + 12 + 11 + 4 + 1, 18 + 12 + 11 + 4 + 2 + 1,
18 + 12 + 11 + 4 + 3 + 1, 18 + 12 + 11 + 5 + 1, 18 + 12 + 11 + 5 + 2 + 1, 18 + 12 + 11 + 5 + 3 + 1, 18 + 12 + 11 + 6 + 1, 19 + 12 + 1,
19 + 12 + 2 + 1, 19 + 12 + 3 + 1, 19 + 12 + 4 + 1, 19 + 12 + 4 + 2 + 1, 19 + 12 + 4 + 3 + 1, 19 + 12 + 5 + 1, 19 + 12 + 5 + 2 + 1,
19 + 12 + 5 + 3 + 1, 19 + 12 + 6 + 1, 19 + 12 + 11 + 1, 19 + 12 + 11 + 2 + 1, 19 + 12 + 11 + 3 + 1, 19 + 12 + 11 + 4 + 1, 19 + 12 + 11 + 4 + 2 + 1,
19 + 12 + 11 + 4 + 3 + 1, 19 + 12 + 11 + 5 + 1, 19 + 12 + 11 + 5 + 2 + 1, 19 + 12 + 11 + 5 + 3 + 1, 19 + 12 + 11 + 6 + 1, 20 + 12 + 1,
20 + 12 + 2 + 1, 20 + 12 + 3 + 1, 20 + 12 + 4 + 1, 20 + 12 + 4 + 2 + 1, 20 + 12 + 4 + 3 + 1, 20 + 12 + 5 + 1, 20 + 12 + 5 + 2 + 1,
20 + 12 + 5 + 3 + 1, 20 + 12 + 6 + 1, 20 + 12 + 11 + 1, 20 + 12 + 11 + 2 + 1, 20 + 12 + 11 + 3 + 1, 20 + 12 + 11 + 4 + 1, 20 + 12 + 11 + 4 + 2 + 1,
20 + 12 + 11 + 4 + 3 + 1, 20 + 12 + 11 + 5 + 1, 20 + 12 + 11 + 5 + 2 + 1, 20 + 12 + 11 + 5 + 3 + 1, 20 + 12 + 11 + 6 + 1, 21 + 12 + 1,
21 + 12 + 2 + 1, 21 + 12 + 3 + 1, 21 + 12 + 4 + 1, 21 + 12 + 4 + 2 + 1, 21 + 12 + 4 + 3 + 1, 21 + 12 + 5 + 1, 21 + 12 + 5 + 2 + 1,
21 + 12 + 5 + 3 + 1, 21 + 12 + 6 + 1, 21 + 12 + 11 + 1, 21 + 12 + 11 + 2 + 1, 21 + 12 + 11 + 3 + 1, 21 + 12 + 11 + 4 + 1, 21 + 12 + 11 + 4 + 2 + 1,
21 + 12 + 11 + 4 + 3 + 1, 21 + 12 + 11 + 5 + 1, 21 + 12 + 11 + 5 + 2 + 1, 21 + 12 + 11 + 5 + 3 + 1, 21 + 12 + 11 + 6 + 1, 22 + 12 + 1,
22 + 12 + 2 + 1, 22 + 12 + 3 + 1, 22 + 12 + 4 + 1, 22 + 12 + 4 + 2 + 1, 22 + 12 + 4 + 3 + 1, 22 + 12 + 5 + 1, 22 + 12 + 5 + 2 + 1,
22 + 12 + 5 + 3 + 1, 22 + 12 + 6 + 1, 22 + 12 + 11 + 1, 22 + 12 + 11 + 2 + 1, 22 + 12 + 11 + 3 + 1, 22 + 12 + 11 + 4 + 1, 22 + 12 + 11 + 4 + 2 + 1,
22 + 12 + 11 + 4 + 3 + 1, 22 + 12 + 11 + 5 + 1, 22 + 12 + 11 + 5 + 2 + 1, 22 + 12 + 11 + 5 + 3 + 1, 22 + 12 + 11 + 6 + 1, 29 + 22 + 13 + 1,
29 + 22 + 13 + 2 + 1, 29 + 22 + 13 + 3 + 1, 29 + 22 + 13 + 4 + 1, 29 + 22 + 13 + 4 + 2 + 1, 29 + 22 + 13 + 4 + 3 + 1, 29 + 22 + 13 + 5 + 1,
29 + 22 + 13 + 5 + 2 + 1, 29 + 22 + 13 + 5 + 3 + 1, 29 + 22 + 13 + 6 + 1, 29 + 22 + 13 + 11 + 1, 29 + 22 + 13 + 11 + 2 + 1,
29 + 22 + 13 + 11 + 3 + 1, 29 + 22 + 13 + 11 + 4 + 1, 29 + 22 + 13 + 11 + 4 + 2 + 1, 29 + 22 + 13 + 11 + 4 + 3 + 1, 29 + 22 + 13 + 11 + 5 + 1,
29 + 22 + 13 + 11 + 5 + 2 + 1, 29 + 22 + 13 + 11 + 5 + 3 + 1, 29 + 22 + 13 + 11 + 6 + 1, 29 + 22 + 14 + 1, 29 + 22 + 14 + 2 + 1,
29 + 22 + 14 + 3 + 1, 29 + 22 + 14 + 4 + 1, 29 + 22 + 14 + 4 + 2 + 1, 29 + 22 + 14 + 4 + 3 + 1, 29 + 22 + 14 + 5 + 1, 29 + 22 + 14 + 5 + 2 + 1,
29 + 22 + 14 + 5 + 3 + 1, 29 + 22 + 14 + 6 + 1, 29 + 22 + 14 + 11 + 1, 29 + 22 + 14 + 11 + 2 + 1, 29 + 22 + 14 + 11 + 3 + 1,
29 + 22 + 14 + 11 + 4 + 1, 29 + 22 + 14 + 11 + 4 + 2 + 1, 29 + 22 + 14 + 11 + 4 + 3 + 1, 29 + 22 + 14 + 11 + 5 + 1, 29 + 22 + 14 + 11 + 5 + 2 + 1,
29 + 22 + 14 + 11 + 5 + 3 + 1, 29 + 22 + 14 + 11 + 6 + 1, 29 + 22 + 15 + 1, 29 + 22 + 15 + 2 + 1, 29 + 22 + 15 + 3 + 1, 29 + 22 + 15 + 4 + 1,
29 + 22 + 15 + 4 + 2 + 1, 29 + 22 + 15 + 4 + 3 + 1, 29 + 22 + 15 + 5 + 1, 29 + 22 + 15 + 5 + 2 + 1, 29 + 22 + 15 + 5 + 3 + 1, 29 + 22 + 15 + 6 + 1,
29 + 22 + 15 + 11 + 1, 29 + 22 + 15 + 11 + 2 + 1, 29 + 22 + 15 + 11 + 3 + 1, 29 + 22 + 15 + 11 + 4 + 1, 29 + 22 + 15 + 11 + 4 + 2 + 1,
29 + 22 + 15 + 11 + 4 + 3 + 1, 29 + 22 + 15 + 11 + 5 + 1, 29 + 22 + 15 + 11 + 5 + 2 + 1, 29 + 22 + 15 + 11 + 5 + 3 + 1, 29 + 22 + 15 + 11 + 6 + 1,
22 + 16 + 1, 22 + 16 + 2 + 1, 22 + 16 + 3 + 1, 22 + 16 + 4 + 1, 22 + 16 + 4 + 2 + 1, 22 + 16 + 4 + 3 + 1, 22 + 16 + 5 + 1, 22 + 16 + 5 + 2 + 1,
22 + 16 + 5 + 3 + 1, 22 + 16 + 6 + 1, 22 + 16 + 11 + 1, 22 + 16 + 11 + 2 + 1, 22 + 16 + 11 + 3 + 1, 22 + 16 + 11 + 4 + 1, 22 + 16 + 11 + 4 + 2 + 1,
22 + 16 + 11 + 4 + 3 + 1, 22 + 16 + 11 + 5 + 1, 22 + 16 + 11 + 5 + 2 + 1, 22 + 16 + 11 + 5 + 3 + 1, 22 + 16 + 11 + 6 + 1, 22 + 17 + 1,
22 + 17 + 2 + 1, 22 + 17 + 3 + 1, 22 + 17 + 4 + 1, 22 + 17 + 4 + 2 + 1, 22 + 17 + 4 + 3 + 1, 22 + 17 + 5 + 1, 22 + 17 + 5 + 2 + 1,
22 + 17 + 5 + 3 + 1, 22 + 17 + 6 + 1, 22 + 17 + 11 + 1, 22 + 17 + 11 + 2 + 1, 22 + 17 + 11 + 3 + 1, 22 + 17 + 11 + 4 + 1, 22 + 17 + 11 + 4 + 2 + 1,
22 + 17 + 11 + 4 + 3 + 1, 22 + 17 + 11 + 5 + 1, 22 + 17 + 11 + 5 + 2 + 1, 22 + 17 + 11 + 5 + 3 + 1, 22 + 17 + 11 + 6 + 1, 22 + 18 + 1,
22 + 18 + 2 + 1, 22 + 18 + 3 + 1, 22 + 18 + 4 + 1, 22 + 18 + 4 + 2 + 1, 22 + 18 + 4 + 3 + 1, 22 + 18 + 5 + 1, 22 + 18 + 5 + 2 + 1,
22 + 18 + 5 + 3 + 1, 22 + 18 + 6 + 1, 22 + 18 + 11 + 1, 22 + 18 + 11 + 2 + 1, 22 + 18 + 11 + 3 + 1, 22 + 18 + 11 + 4 + 1, 22 + 18 + 11 + 4 + 2 + 1,
22 + 18 + 11 + 4 + 3 + 1, 22 + 18 + 11 + 5 + 1, 22 + 18 + 11 + 5 + 2 + 1, 22 + 18 + 11 + 5 + 3 + 1, 22 + 18 + 11 + 6 + 1, 22 + 19 + 1,
22 + 19 + 2 + 1, 22 + 19 + 3 + 1, 22 + 19 + 4 + 1, 22 + 19 + 4 + 2 + 1, 22 + 19 + 4 + 3 + 1, 22 + 19 + 5 + 1, 22 + 19 + 5 + 2 + 1,
22 + 19 + 5 + 3 + 1, 22 + 19 + 6 + 1, 22 + 19 + 11 + 1, 22 + 19 + 11 + 2 + 1, 22 + 19 + 11 + 3 + 1, 22 + 19 + 11 + 4 + 1, 22 + 19 + 11 + 4 + 2 + 1,
22 + 19 + 11 + 4 + 3 + 1, 22 + 19 + 11 + 5 + 1, 22 + 19 + 11 + 5 + 2 + 1, 22 + 19 + 11 + 5 + 3 + 1, 22 + 19 + 11 + 6 + 1, 22 + 20 + 1,
22 + 20 + 2 + 1, 22 + 20 + 3 + 1, 22 + 20 + 4 + 1, 22 + 20 + 4 + 2 + 1, 22 + 20 + 4 + 3 + 1, 22 + 20 + 5 + 1, 22 + 20 + 5 + 2 + 1,
22 + 20 + 5 + 3 + 1, 22 + 20 + 6 + 1, 22 + 20 + 11 + 1, 22 + 20 + 11 + 2 + 1, 22 + 20 + 11 + 3 + 1, 22 + 20 + 11 + 4 + 1, 22 + 20 + 11 + 4 + 2 + 1,
22 + 20 + 11 + 4 + 3 + 1, 22 + 20 + 11 + 5 + 1, 22 + 20 + 11 + 5 + 2 + 1, 22 + 20 + 11 + 5 + 3 + 1, 22 + 20 + 11 + 6 + 1, 22 + 21 + 1,
22 + 21 + 2 + 1, 22 + 21 + 3 + 1, 22 + 21 + 4 + 1, 22 + 21 + 4 + 2 + 1, 22 + 21 + 4 + 3 + 1, 22 + 21 + 5 + 1, 22 + 21 + 5 + 2 + 1,
22 + 21 + 5 + 3 + 1, 22 + 21 + 6 + 1, 22 + 21 + 11 + 1, 22 + 21 + 11 + 2 + 1, 22 + 21 + 11 + 3 + 1, 22 + 21 + 11 + 4 + 1, 22 + 21 + 11 + 4 + 2 + 1,
22 + 21 + 11 + 4 + 3 + 1, 22 + 21 + 11 + 5 + 1, 22 + 21 + 11 + 5 + 2 + 1, 22 + 21 + 11 + 5 + 3 + 1, 22 + 21 + 11 + 6 + 1, 23 + 12 + 1,
23 + 12 + 2 + 1, 23 + 12 + 3 + 1, 23 + 12 + 4 + 1, 23 + 12 + 4 + 2 + 1, 23 + 12 + 4 + 3 + 1, 23 + 12 + 5 + 1, 23 + 12 + 5 + 2 + 1,
23 + 12 + 5 + 3 + 1, 23 + 12 + 6 + 1, 23 + 12 + 11 + 1, 23 + 12 + 11 + 2 + 1, 23 + 12 + 11 + 3 + 1, 23 + 12 + 11 + 4 + 1, 23 + 12 + 11 + 4 + 2 + 1,
23 + 12 + 11 + 4 + 3 + 1, 23 + 12 + 11 + 5 + 1, 23 + 12 + 11 + 5 + 2 + 1, 23 + 12 + 11 + 5 + 3 + 1, 23 + 12 + 11 + 6 + 1, 23 + 13 + 1,
23 + 13 + 2 + 1, 23 + 13 + 3 + 1, 23 + 13 + 4 + 1, 23 + 13 + 4 + 2 + 1, 23 + 13 + 4 + 3 + 1, 23 + 13 + 5 + 1, 23 + 13 + 5 + 2 + 1,
23 + 13 + 5 + 3 + 1, 23 + 13 + 6 + 1, 23 + 13 + 11 + 1, 23 + 13 + 11 + 2 + 1, 23 + 13 + 11 + 3 + 1, 23 + 13 + 11 + 4 + 1, 23 + 13 + 11 + 4 + 2 + 1,
23 + 13 + 11 + 4 + 3 + 1, 23 + 13 + 11 + 5 + 1, 23 + 13 + 11 + 5 + 2 + 1, 23 + 13 + 11 + 5 + 3 + 1, 23 + 13 + 11 + 6 + 1, 23 + 14 + 1,
23 + 14 + 2 + 1, 23 + 14 + 3 + 1, 23 + 14 + 4 + 1, 23 + 14 + 4 + 2 + 1, 23 + 14 + 4 + 3 + 1, 23 + 14 + 5 + 1, 23 + 14 + 5 + 2 + 1,
23 + 14 + 5 + 3 + 1, 23 + 14 + 6 + 1, 23 + 14 + 11 + 1, 23 + 14 + 11 + 2 + 1, 23 + 14 + 11 + 3 + 1, 23 + 14 + 11 + 4 + 1, 23 + 14 + 11 + 4 + 2 + 1,
23 + 14 + 11 + 4 + 3 + 1, 23 + 14 + 11 + 5 + 1, 23 + 14 + 11 + 5 + 2 + 1, 23 + 14 + 11 + 5 + 3 + 1, 23 + 14 + 11 + 6 + 1, 23 + 15 + 1,
23 + 15 + 2 + 1, 23 + 15 + 3 + 1, 23 + 15 + 4 + 1, 23 + 15 + 4 + 2 + 1, 23 + 15 + 4 + 3 + 1, 23 + 15 + 5 + 1, 23 + 15 + 5 + 2 + 1,
23 + 15 + 5 + 3 + 1, 23 + 15 + 6 + 1, 23 + 15 + 11 + 1, 23 + 15 + 11 + 2 + 1, 23 + 15 + 11 + 3 + 1, 23 + 15 + 11 + 4 + 1, 23 + 15 + 11 + 4 + 2 + 1,
23 + 15 + 11 + 4 + 3 + 1, 23 + 15 + 11 + 5 + 1, 23 + 15 + 11 + 5 + 2 + 1, 23 + 15 + 11 + 5 + 3 + 1, 23 + 15 + 11 + 6 + 1, 23 + 16 + 1,
23 + 16 + 2 + 1, 23 + 16 + 3 + 1, 23 + 16 + 4 + 1, 23 + 16 + 4 + 2 + 1, 23 + 16 + 4 + 3 + 1, 23 + 16 + 5 + 1, 23 + 16 + 5 + 2 + 1,
23 + 16 + 5 + 3 + 1, 23 + 16 + 6 + 1, 23 + 16 + 11 + 1, 23 + 16 + 11 + 2 + 1, 23 + 16 + 11 + 3 + 1, 23 + 16 + 11 + 4 + 1, 23 + 16 + 11 + 4 + 2 + 1,
23 + 16 + 11 + 4 + 3 + 1, 23 + 16 + 11 + 5 + 1, 23 + 16 + 11 + 5 + 2 + 1, 23 + 16 + 11 + 5 + 3 + 1, 23 + 16 + 11 + 6 + 1, 23 + 17 + 1,
23 + 17 + 2 + 1, 23 + 17 + 3 + 1, 23 + 17 + 4 + 1, 23 + 17 + 4 + 2 + 1, 23 + 17 + 4 + 3 + 1, 23 + 17 + 5 + 1, 23 + 17 + 5 + 2 + 1,
23 + 17 + 5 + 3 + 1, 23 + 17 + 6 + 1, 23 + 17 + 11 + 1, 23 + 17 + 11 + 2 + 1, 23 + 17 + 11 + 3 + 1, 23 + 17 + 11 + 4 + 1, 23 + 17 + 11 + 4 + 2 + 1,
23 + 17 + 11 + 4 + 3 + 1, 23 + 17 + 11 + 5 + 1, 23 + 17 + 11 + 5 + 2 + 1, 23 + 17 + 11 + 5 + 3 + 1, 23 + 17 + 11 + 6 + 1, 23 + 18 + 1,
23 + 18 + 2 + 1, 23 + 18 + 3 + 1, 23 + 18 + 4 + 1, 23 + 18 + 4 + 2 + 1, 23 + 18 + 4 + 3 + 1, 23 + 18 + 5 + 1, 23 + 18 + 5 + 2 + 1,
23 + 18 + 5 + 3 + 1, 23 + 18 + 6 + 1, 23 + 18 + 11 + 1, 23 + 18 + 11 + 2 + 1, 23 + 18 + 11 + 3 + 1, 23 + 18 + 11 + 4 + 1, 23 + 18 + 11 + 4 + 2 + 1,
23 + 18 + 11 + 4 + 3 + 1, 23 + 18 + 11 + 5 + 1, 23 + 18 + 11 + 5 + 2 + 1, 23 + 18 + 11 + 5 + 3 + 1, 23 + 18 + 11 + 6 + 1, 23 + 19 + 1,
23 + 19 + 2 + 1, 23 + 19 + 3 + 1, 23 + 19 + 4 + 1, 23 + 19 + 4 + 2 + 1, 23 + 19 + 4 + 3 + 1, 23 + 19 + 5 + 1, 23 + 19 + 5 + 2 + 1,
23 + 19 + 5 + 3 + 1, 23 + 19 + 6 + 1, 23 + 19 + 11 + 1, 23 + 19 + 11 + 2 + 1, 23 + 19 + 11 + 3 + 1, 23 + 19 + 11 + 4 + 1, 23 + 19 + 11 + 4 + 2 + 1,
23 + 19 + 11 + 4 + 3 + 1, 23 + 19 + 11 + 5 + 1, 23 + 19 + 11 + 5 + 2 + 1, 23 + 19 + 11 + 5 + 3 + 1, 23 + 19 + 11 + 6 + 1, 23 + 20 + 1,
23 + 20 + 2 + 1, 23 + 20 + 3 + 1, 23 + 20 + 4 + 1, 23 + 20 + 4 + 2 + 1, 23 + 20 + 4 + 3 + 1, 23 + 20 + 5 + 1, 23 + 20 + 5 + 2 + 1,
23 + 20 + 5 + 3 + 1, 23 + 20 + 6 + 1, 23 + 20 + 11 + 1, 23 + 20 + 11 + 2 + 1, 23 + 20 + 11 + 3 + 1, 23 + 20 + 11 + 4 + 1, 23 + 20 + 11 + 4 + 2 + 1,
23 + 20 + 11 + 4 + 3 + 1, 23 + 20 + 11 + 5 + 1, 23 + 20 + 11 + 5 + 2 + 1, 23 + 20 + 11 + 5 + 3 + 1, 23 + 20 + 11 + 6 + 1, 23 + 21 + 1,
23 + 21 + 2 + 1, 23 + 21 + 3 + 1, 23 + 21 + 4 + 1, 23 + 21 + 4 + 2 + 1, 23 + 21 + 4 + 3 + 1, 23 + 21 + 5 + 1, 23 + 21 + 5 + 2 + 1,
23 + 21 + 5 + 3 + 1, 23 + 21 + 6 + 1, 23 + 21 + 11 + 1, 23 + 21 + 11 + 2 + 1, 23 + 21 + 11 + 3 + 1, 23 + 21 + 11 + 4 + 1, 23 + 21 + 11 + 4 + 2 + 1,
23 + 21 + 11 + 4 + 3 + 1, 23 + 21 + 11 + 5 + 1, 23 + 21 + 11 + 5 + 2 + 1, 23 + 21 + 11 + 5 + 3 + 1, 23 + 21 + 11 + 6 + 1, 24 + 12 + 1,
24 + 12 + 2 + 1, 24 + 12 + 3 + 1, 24 + 12 + 4 + 1, 24 + 12 + 4 + 2 + 1, 24 + 12 + 4 + 3 + 1, 24 + 12 + 5 + 1, 24 + 12 + 5 + 2 + 1,
24 + 12 + 5 + 3 + 1, 24 + 12 + 6 + 1, 24 + 12 + 11 + 1, 24 + 12 + 11 + 2 + 1, 24 + 12 + 11 + 3 + 1, 24 + 12 + 11 + 4 + 1, 24 + 12 + 11 + 4 + 2 + 1,
24 + 12 + 11 + 4 + 3 + 1, 24 + 12 + 11 + 5 + 1, 24 + 12 + 11 + 5 + 2 + 1, 24 + 12 + 11 + 5 + 3 + 1, 24 + 12 + 11 + 6 + 1, 24 + 13 + 1,
24 + 13 + 2 + 1, 24 + 13 + 3 + 1, 24 + 13 + 4 + 1, 24 + 13 + 4 + 2 + 1, 24 + 13 + 4 + 3 + 1, 24 + 13 + 5 + 1, 24 + 13 + 5 + 2 + 1,
24 + 13 + 5 + 3 + 1, 24 + 13 + 6 + 1, 24 + 13 + 11 + 1, 24 + 13 + 11 + 2 + 1, 24 + 13 + 11 + 3 + 1, 24 + 13 + 11 + 4 + 1, 24 + 13 + 11 + 4 + 2 + 1,
24 + 13 + 11 + 4 + 3 + 1, 24 + 13 + 11 + 5 + 1, 24 + 13 + 11 + 5 + 2 + 1, 24 + 13 + 11 + 5 + 3 + 1, 24 + 13 + 11 + 6 + 1, 24 + 14 + 1,
24 + 14 + 2 + 1, 24 + 14 + 3 + 1, 24 + 14 + 4 + 1, 24 + 14 + 4 + 2 + 1, 24 + 14 + 4 + 3 + 1, 24 + 14 + 5 + 1, 24 + 14 + 5 + 2 + 1,
24 + 14 + 5 + 3 + 1, 24 + 14 + 6 + 1, 24 + 14 + 11 + 1, 24 + 14 + 11 + 2 + 1, 24 + 14 + 11 + 3 + 1, 24 + 14 + 11 + 4 + 1, 24 + 14 + 11 + 4 + 2 + 1,
24 + 14 + 11 + 4 + 3 + 1, 24 + 14 + 11 + 5 + 1, 24 + 14 + 11 + 5 + 2 + 1, 24 + 14 + 11 + 5 + 3 + 1, 24 + 14 + 11 + 6 + 1, 24 + 15 + 1,
24 + 15 + 2 + 1, 24 + 15 + 3 + 1, 24 + 15 + 4 + 1, 24 + 15 + 4 + 2 + 1, 24 + 15 + 4 + 3 + 1, 24 + 15 + 5 + 1, 24 + 15 + 5 + 2 + 1,
24 + 15 + 5 + 3 + 1, 24 + 15 + 6 + 1, 24 + 15 + 11 + 1, 24 + 15 + 11 + 2 + 1, 24 + 15 + 11 + 3 + 1, 24 + 15 + 11 + 4 + 1, 24 + 15 + 11 + 4 + 2 + 1,
24 + 15 + 11 + 4 + 3 + 1, 24 + 15 + 11 + 5 + 1, 24 + 15 + 11 + 5 + 2 + 1, 24 + 15 + 11 + 5 + 3 + 1, 24 + 15 + 11 + 6 + 1, 24 + 16 + 1,
24 + 16 + 2 + 1, 24 + 16 + 3 + 1, 24 + 16 + 4 + 1, 24 + 16 + 4 + 2 + 1, 24 + 16 + 4 + 3 + 1, 24 + 16 + 5 + 1, 24 + 16 + 5 + 2 + 1,
24 + 16 + 5 + 3 + 1, 24 + 16 + 6 + 1, 24 + 16 + 11 + 1, 24 + 16 + 11 + 2 + 1, 24 + 16 + 11 + 3 + 1, 24 + 16 + 11 + 4 + 1, 24 + 16 + 11 + 4 + 2 + 1,
24 + 16 + 11 + 4 + 3 + 1, 24 + 16 + 11 + 5 + 1, 24 + 16 + 11 + 5 + 2 + 1, 24 + 16 + 11 + 5 + 3 + 1, 24 + 16 + 11 + 6 + 1, 24 + 17 + 1,
24 + 17 + 2 + 1, 24 + 17 + 3 + 1, 24 + 17 + 4 + 1, 24 + 17 + 4 + 2 + 1, 24 + 17 + 4 + 3 + 1, 24 + 17 + 5 + 1, 24 + 17 + 5 + 2 + 1,
24 + 17 + 5 + 3 + 1, 24 + 17 + 6 + 1, 24 + 17 + 11 + 1, 24 + 17 + 11 + 2 + 1, 24 + 17 + 11 + 3 + 1, 24 + 17 + 11 + 4 + 1, 24 + 17 + 11 + 4 + 2 + 1,
24 + 17 + 11 + 4 + 3 + 1, 24 + 17 + 11 + 5 + 1, 24 + 17 + 11 + 5 + 2 + 1, 24 + 17 + 11 + 5 + 3 + 1, 24 + 17 + 11 + 6 + 1, 24 + 18 + 1,
24 + 18 + 2 + 1, 24 + 18 + 3 + 1, 24 + 18 + 4 + 1, 24 + 18 + 4 + 2 + 1, 24 + 18 + 4 + 3 + 1, 24 + 18 + 5 + 1, 24 + 18 + 5 + 2 + 1,
24 + 18 + 5 + 3 + 1, 24 + 18 + 6 + 1, 24 + 18 + 11 + 1, 24 + 18 + 11 + 2 + 1, 24 + 18 + 11 + 3 + 1, 24 + 18 + 11 + 4 + 1, 24 + 18 + 11 + 4 + 2 + 1,
24 + 18 + 11 + 4 + 3 + 1, 24 + 18 + 11 + 5 + 1, 24 + 18 + 11 + 5 + 2 + 1, 24 + 18 + 11 + 5 + 3 + 1, 24 + 18 + 11 + 6 + 1, 24 + 19 + 1,
24 + 19 + 2 + 1, 24 + 19 + 3 + 1, 24 + 19 + 4 + 1, 24 + 19 + 4 + 2 + 1, 24 + 19 + 4 + 3 + 1, 24 + 19 + 5 + 1, 24 + 19 + 5 + 2 + 1,
24 + 19 + 5 + 3 + 1, 24 + 19 + 6 + 1, 24 + 19 + 11 + 1, 24 + 19 + 11 + 2 + 1, 24 + 19 + 11 + 3 + 1, 24 + 19 + 11 + 4 + 1, 24 + 19 + 11 + 4 + 2 + 1,
24 + 19 + 11 + 4 + 3 + 1, 24 + 19 + 11 + 5 + 1, 24 + 19 + 11 + 5 + 2 + 1, 24 + 19 + 11 + 5 + 3 + 1, 24 + 19 + 11 + 6 + 1, 24 + 20 + 1,
24 + 20 + 2 + 1, 24 + 20 + 3 + 1, 24 + 20 + 4 + 1, 24 + 20 + 4 + 2 + 1, 24 + 20 + 4 + 3 + 1, 24 + 20 + 5 + 1, 24 + 20 + 5 + 2 + 1,
24 + 20 + 5 + 3 + 1, 24 + 20 + 6 + 1, 24 + 20 + 11 + 1, 24 + 20 + 11 + 2 + 1, 24 + 20 + 11 + 3 + 1, 24 + 20 + 11 + 4 + 1, 24 + 20 + 11 + 4 + 2 + 1,
24 + 20 + 11 + 4 + 3 + 1, 24 + 20 + 11 + 5 + 1, 24 + 20 + 11 + 5 + 2 + 1, 24 + 20 + 11 + 5 + 3 + 1, 24 + 20 + 11 + 6 + 1, 24 + 21 + 1,
24 + 21 + 2 + 1, 24 + 21 + 3 + 1, 24 + 21 + 4 + 1, 24 + 21 + 4 + 2 + 1, 24 + 21 + 4 + 3 + 1, 24 + 21 + 5 + 1, 24 + 21 + 5 + 2 + 1,
24 + 21 + 5 + 3 + 1, 24 + 21 + 6 + 1, 24 + 21 + 11 + 1, 24 + 21 + 11 + 2 + 1, 24 + 21 + 11 + 3 + 1, 24 + 21 + 11 + 4 + 1, 24 + 21 + 11 + 4 + 2 + 1,
24 + 21 + 11 + 4 + 3 + 1, 24 + 21 + 11 + 5 + 1, 24 + 21 + 11 + 5 + 2 + 1, 24 + 21 + 11 + 5 + 3 + 1, 24 + 21 + 11 + 6 + 1, 25 + 12 + 1,
25 + 12 + 2 + 1, 25 + 12 + 3 + 1, 25 + 12 + 4 + 1, 25 + 12 + 4 + 2 + 1, 25 + 12 + 4 + 3 + 1, 25 + 12 + 5 + 1, 25 + 12 + 5 + 2 + 1,
25 + 12 + 5 + 3 + 1, 25 + 12 + 6 + 1, 25 + 12 + 11 + 1, 25 + 12 + 11 + 2 + 1, 25 + 12 + 11 + 3 + 1, 25 + 12 + 11 + 4 + 1, 25 + 12 + 11 + 4 + 2 + 1,
25 + 12 + 11 + 4 + 3 + 1, 25 + 12 + 11 + 5 + 1, 25 + 12 + 11 + 5 + 2 + 1, 25 + 12 + 11 + 5 + 3 + 1, 25 + 12 + 11 + 6 + 1, 25 + 13 + 1,
25 + 13 + 2 + 1, 25 + 13 + 3 + 1, 25 + 13 + 4 + 1, 25 + 13 + 4 + 2 + 1, 25 + 13 + 4 + 3 + 1, 25 + 13 + 5 + 1, 25 + 13 + 5 + 2 + 1,
25 + 13 + 5 + 3 + 1, 25 + 13 + 6 + 1, 25 + 13 + 11 + 1, 25 + 13 + 11 + 2 + 1, 25 + 13 + 11 + 3 + 1, 25 + 13 + 11 + 4 + 1, 25 + 13 + 11 + 4 + 2 + 1,
25 + 13 + 11 + 4 + 3 + 1, 25 + 13 + 11 + 5 + 1, 25 + 13 + 11 + 5 + 2 + 1, 25 + 13 + 11 + 5 + 3 + 1, 25 + 13 + 11 + 6 + 1, 25 + 14 + 1,
25 + 14 + 2 + 1, 25 + 14 + 3 + 1, 25 + 14 + 4 + 1, 25 + 14 + 4 + 2 + 1, 25 + 14 + 4 + 3 + 1, 25 + 14 + 5 + 1, 25 + 14 + 5 + 2 + 1,
25 + 14 + 5 + 3 + 1, 25 + 14 + 6 + 1, 25 + 14 + 11 + 1, 25 + 14 + 11 + 2 + 1, 25 + 14 + 11 + 3 + 1, 25 + 14 + 11 + 4 + 1, 25 + 14 + 11 + 4 + 2 + 1,
25 + 14 + 11 + 4 + 3 + 1, 25 + 14 + 11 + 5 + 1, 25 + 14 + 11 + 5 + 2 + 1, 25 + 14 + 11 + 5 + 3 + 1, 25 + 14 + 11 + 6 + 1, 25 + 15 + 1,
25 + 15 + 2 + 1, 25 + 15 + 3 + 1, 25 + 15 + 4 + 1, 25 + 15 + 4 + 2 + 1, 25 + 15 + 4 + 3 + 1, 25 + 15 + 5 + 1, 25 + 15 + 5 + 2 + 1,
25 + 15 + 5 + 3 + 1, 25 + 15 + 6 + 1, 25 + 15 + 11 + 1, 25 + 15 + 11 + 2 + 1, 25 + 15 + 11 + 3 + 1, 25 + 15 + 11 + 4 + 1, 25 + 15 + 11 + 4 + 2 + 1,
25 + 15 + 11 + 4 + 3 + 1, 25 + 15 + 11 + 5 + 1, 25 + 15 + 11 + 5 + 2 + 1, 25 + 15 + 11 + 5 + 3 + 1, 25 + 15 + 11 + 6 + 1, 25 + 16 + 1,
25 + 16 + 2 + 1, 25 + 16 + 3 + 1, 25 + 16 + 4 + 1, 25 + 16 + 4 + 2 + 1, 25 + 16 + 4 + 3 + 1, 25 + 16 + 5 + 1, 25 + 16 + 5 + 2 + 1,
25 + 16 + 5 + 3 + 1, 25 + 16 + 6 + 1, 25 + 16 + 11 + 1, 25 + 16 + 11 + 2 + 1, 25 + 16 + 11 + 3 + 1, 25 + 16 + 11 + 4 + 1, 25 + 16 + 11 + 4 + 2 + 1,
25 + 16 + 11 + 4 + 3 + 1, 25 + 16 + 11 + 5 + 1, 25 + 16 + 11 + 5 + 2 + 1, 25 + 16 + 11 + 5 + 3 + 1, 25 + 16 + 11 + 6 + 1, 25 + 17 + 1,
25 + 17 + 2 + 1, 25 + 17 + 3 + 1, 25 + 17 + 4 + 1, 25 + 17 + 4 + 2 + 1, 25 + 17 + 4 + 3 + 1, 25 + 17 + 5 + 1, 25 + 17 + 5 + 2 + 1,
25 + 17 + 5 + 3 + 1, 25 + 17 + 6 + 1, 25 + 17 + 11 + 1, 25 + 17 + 11 + 2 + 1, 25 + 17 + 11 + 3 + 1, 25 + 17 + 11 + 4 + 1, 25 + 17 + 11 + 4 + 2 + 1,
25 + 17 + 11 + 4 + 3 + 1, 25 + 17 + 11 + 5 + 1, 25 + 17 + 11 + 5 + 2 + 1, 25 + 17 + 11 + 5 + 3 + 1, 25 + 17 + 11 + 6 + 1, 25 + 18 + 1,
25 + 18 + 2 + 1, 25 + 18 + 3 + 1, 25 + 18 + 4 + 1, 25 + 18 + 4 + 2 + 1, 25 + 18 + 4 + 3 + 1, 25 + 18 + 5 + 1, 25 + 18 + 5 + 2 + 1,
25 + 18 + 5 + 3 + 1, 25 + 18 + 6 + 1, 25 + 18 + 11 + 1, 25 + 18 + 11 + 2 + 1, 25 + 18 + 11 + 3 + 1, 25 + 18 + 11 + 4 + 1, 25 + 18 + 11 + 4 + 2 + 1,
25 + 18 + 11 + 4 + 3 + 1, 25 + 18 + 11 + 5 + 1, 25 + 18 + 11 + 5 + 2 + 1, 25 + 18 + 11 + 5 + 3 + 1, 25 + 18 + 11 + 6 + 1, 25 + 19 + 1,
25 + 19 + 2 + 1, 25 + 19 + 3 + 1, 25 + 19 + 4 + 1, 25 + 19 + 4 + 2 + 1, 25 + 19 + 4 + 3 + 1, 25 + 19 + 5 + 1, 25 + 19 + 5 + 2 + 1,
25 + 19 + 5 + 3 + 1, 25 + 19 + 6 + 1, 25 + 19 + 11 + 1, 25 + 19 + 11 + 2 + 1, 25 + 19 + 11 + 3 + 1, 25 + 19 + 11 + 4 + 1, 25 + 19 + 11 + 4 + 2 + 1,
25 + 19 + 11 + 4 + 3 + 1, 25 + 19 + 11 + 5 + 1, 25 + 19 + 11 + 5 + 2 + 1, 25 + 19 + 11 + 5 + 3 + 1, 25 + 19 + 11 + 6 + 1, 25 + 20 + 1,
25 + 20 + 2 + 1, 25 + 20 + 3 + 1, 25 + 20 + 4 + 1, 25 + 20 + 4 + 2 + 1, 25 + 20 + 4 + 3 + 1, 25 + 20 + 5 + 1, 25 + 20 + 5 + 2 + 1,
25 + 20 + 5 + 3 + 1, 25 + 20 + 6 + 1, 25 + 20 + 11 + 1, 25 + 20 + 11 + 2 + 1, 25 + 20 + 11 + 3 + 1, 25 + 20 + 11 + 4 + 1, 25 + 20 + 11 + 4 + 2 + 1,
25 + 20 + 11 + 4 + 3 + 1, 25 + 20 + 11 + 5 + 1, 25 + 20 + 11 + 5 + 2 + 1, 25 + 20 + 11 + 5 + 3 + 1, 25 + 20 + 11 + 6 + 1, 25 + 21 + 1,
25 + 21 + 2 + 1, 25 + 21 + 3 + 1, 25 + 21 + 4 + 1, 25 + 21 + 4 + 2 + 1, 25 + 21 + 4 + 3 + 1, 25 + 21 + 5 + 1, 25 + 21 + 5 + 2 + 1,
25 + 21 + 5 + 3 + 1, 25 + 21 + 6 + 1, 25 + 21 + 11 + 1, 25 + 21 + 11 + 2 + 1, 25 + 21 + 11 + 3 + 1, 25 + 21 + 11 + 4 + 1, 25 + 21 + 11 + 4 + 2 + 1,
25 + 21 + 11 + 4 + 3 + 1, 25 + 21 + 11 + 5 + 1, 25 + 21 + 11 + 5 + 2 + 1, 25 + 21 + 11 + 5 + 3 + 1, 25 + 21 + 11 + 6 + 1, 26 + 12 + 1,
26 + 12 + 2 + 1, 26 + 12 + 3 + 1, 26 + 12 + 4 + 1, 26 + 12 + 4 + 2 + 1, 26 + 12 + 4 + 3 + 1, 26 + 12 + 5 + 1, 26 + 12 + 5 + 2 + 1,
26 + 12 + 5 + 3 + 1, 26 + 12 + 6 + 1, 26 + 12 + 11 + 1, 26 + 12 + 11 + 2 + 1, 26 + 12 + 11 + 3 + 1, 26 + 12 + 11 + 4 + 1, 26 + 12 + 11 + 4 + 2 + 1,
26 + 12 + 11 + 4 + 3 + 1, 26 + 12 + 11 + 5 + 1, 26 + 12 + 11 + 5 + 2 + 1, 26 + 12 + 11 + 5 + 3 + 1, 26 + 12 + 11 + 6 + 1, 26 + 13 + 1,
26 + 13 + 2 + 1, 26 + 13 + 3 + 1, 26 + 13 + 4 + 1, 26 + 13 + 4 + 2 + 1, 26 + 13 + 4 + 3 + 1, 26 + 13 + 5 + 1, 26 + 13 + 5 + 2 + 1,
26 + 13 + 5 + 3 + 1, 26 + 13 + 6 + 1, 26 + 13 + 11 + 1, 26 + 13 + 11 + 2 + 1, 26 + 13 + 11 + 3 + 1, 26 + 13 + 11 + 4 + 1, 26 + 13 + 11 + 4 + 2 + 1,
26 + 13 + 11 + 4 + 3 + 1, 26 + 13 + 11 + 5 + 1, 26 + 13 + 11 + 5 + 2 + 1, 26 + 13 + 11 + 5 + 3 + 1, 26 + 13 + 11 + 6 + 1, 26 + 14 + 1,
26 + 14 + 2 + 1, 26 + 14 + 3 + 1, 26 + 14 + 4 + 1, 26 + 14 + 4 + 2 + 1, 26 + 14 + 4 + 3 + 1, 26 + 14 + 5 + 1, 26 + 14 + 5 + 2 + 1,
26 + 14 + 5 + 3 + 1, 26 + 14 + 6 + 1, 26 + 14 + 11 + 1, 26 + 14 + 11 + 2 + 1, 26 + 14 + 11 + 3 + 1, 26 + 14 + 11 + 4 + 1, 26 + 14 + 11 + 4 + 2 + 1,
26 + 14 + 11 + 4 + 3 + 1, 26 + 14 + 11 + 5 + 1, 26 + 14 + 11 + 5 + 2 + 1, 26 + 14 + 11 + 5 + 3 + 1, 26 + 14 + 11 + 6 + 1, 26 + 15 + 1,
26 + 15 + 2 + 1, 26 + 15 + 3 + 1, 26 + 15 + 4 + 1, 26 + 15 + 4 + 2 + 1, 26 + 15 + 4 + 3 + 1, 26 + 15 + 5 + 1, 26 + 15 + 5 + 2 + 1,
26 + 15 + 5 + 3 + 1, 26 + 15 + 6 + 1, 26 + 15 + 11 + 1, 26 + 15 + 11 + 2 + 1, 26 + 15 + 11 + 3 + 1, 26 + 15 + 11 + 4 + 1, 26 + 15 + 11 + 4 + 2 + 1,
26 + 15 + 11 + 4 + 3 + 1, 26 + 15 + 11 + 5 + 1, 26 + 15 + 11 + 5 + 2 + 1, 26 + 15 + 11 + 5 + 3 + 1, 26 + 15 + 11 + 6 + 1, 26 + 16 + 1,
26 + 16 + 2 + 1, 26 + 16 + 3 + 1, 26 + 16 + 4 + 1, 26 + 16 + 4 + 2 + 1, 26 + 16 + 4 + 3 + 1, 26 + 16 + 5 + 1, 26 + 16 + 5 + 2 + 1,
26 + 16 + 5 + 3 + 1, 26 + 16 + 6 + 1, 26 + 16 + 11 + 1, 26 + 16 + 11 + 2 + 1, 26 + 16 + 11 + 3 + 1, 26 + 16 + 11 + 4 + 1, 26 + 16 + 11 + 4 + 2 + 1,
26 + 16 + 11 + 4 + 3 + 1, 26 + 16 + 11 + 5 + 1, 26 + 16 + 11 + 5 + 2 + 1, 26 + 16 + 11 + 5 + 3 + 1, 26 + 16 + 11 + 6 + 1, 26 + 17 + 1,
26 + 17 + 2 + 1, 26 + 17 + 3 + 1, 26 + 17 + 4 + 1, 26 + 17 + 4 + 2 + 1, 26 + 17 + 4 + 3 + 1, 26 + 17 + 5 + 1, 26 + 17 + 5 + 2 + 1,
26 + 17 + 5 + 3 + 1, 26 + 17 + 6 + 1, 26 + 17 + 11 + 1, 26 + 17 + 11 + 2 + 1, 26 + 17 + 11 + 3 + 1, 26 + 17 + 11 + 4 + 1, 26 + 17 + 11 + 4 + 2 + 1,
26 + 17 + 11 + 4 + 3 + 1, 26 + 17 + 11 + 5 + 1, 26 + 17 + 11 + 5 + 2 + 1, 26 + 17 + 11 + 5 + 3 + 1, 26 + 17 + 11 + 6 + 1, 26 + 18 + 1,
26 + 18 + 2 + 1, 26 + 18 + 3 + 1, 26 + 18 + 4 + 1, 26 + 18 + 4 + 2 + 1, 26 + 18 + 4 + 3 + 1, 26 + 18 + 5 + 1, 26 + 18 + 5 + 2 + 1,
26 + 18 + 5 + 3 + 1, 26 + 18 + 6 + 1, 26 + 18 + 11 + 1, 26 + 18 + 11 + 2 + 1, 26 + 18 + 11 + 3 + 1, 26 + 18 + 11 + 4 + 1, 26 + 18 + 11 + 4 + 2 + 1,
26 + 18 + 11 + 4 + 3 + 1, 26 + 18 + 11 + 5 + 1, 26 + 18 + 11 + 5 + 2 + 1, 26 + 18 + 11 + 5 + 3 + 1, 26 + 18 + 11 + 6 + 1, 26 + 19 + 1,
26 + 19 + 2 + 1, 26 + 19 + 3 + 1, 26 + 19 + 4 + 1, 26 + 19 + 4 + 2 + 1, 26 + 19 + 4 + 3 + 1, 26 + 19 + 5 + 1, 26 + 19 + 5 + 2 + 1,
26 + 19 + 5 + 3 + 1, 26 + 19 + 6 + 1, 26 + 19 + 11 + 1, 26 + 19 + 11 + 2 + 1, 26 + 19 + 11 + 3 + 1, 26 + 19 + 11 + 4 + 1, 26 + 19 + 11 + 4 + 2 + 1,
26 + 19 + 11 + 4 + 3 + 1, 26 + 19 + 11 + 5 + 1, 26 + 19 + 11 + 5 + 2 + 1, 26 + 19 + 11 + 5 + 3 + 1, 26 + 19 + 11 + 6 + 1, 26 + 20 + 1,
26 + 20 + 2 + 1, 26 + 20 + 3 + 1, 26 + 20 + 4 + 1, 26 + 20 + 4 + 2 + 1, 26 + 20 + 4 + 3 + 1, 26 + 20 + 5 + 1, 26 + 20 + 5 + 2 + 1,
26 + 20 + 5 + 3 + 1, 26 + 20 + 6 + 1, 26 + 20 + 11 + 1, 26 + 20 + 11 + 2 + 1, 26 + 20 + 11 + 3 + 1, 26 + 20 + 11 + 4 + 1, 26 + 20 + 11 + 4 + 2 + 1,
26 + 20 + 11 + 4 + 3 + 1, 26 + 20 + 11 + 5 + 1, 26 + 20 + 11 + 5 + 2 + 1, 26 + 20 + 11 + 5 + 3 + 1, 26 + 20 + 11 + 6 + 1, 26 + 21 + 1,
26 + 21 + 2 + 1, 26 + 21 + 3 + 1, 26 + 21 + 4 + 1, 26 + 21 + 4 + 2 + 1, 26 + 21 + 4 + 3 + 1, 26 + 21 + 5 + 1, 26 + 21 + 5 + 2 + 1,
26 + 21 + 5 + 3 + 1, 26 + 21 + 6 + 1, 26 + 21 + 11 + 1, 26 + 21 + 11 + 2 + 1, 26 + 21 + 11 + 3 + 1, 26 + 21 + 11 + 4 + 1, 26 + 21 + 11 + 4 + 2 + 1,
26 + 21 + 11 + 4 + 3 + 1, 26 + 21 + 11 + 5 + 1, 26 + 21 + 11 + 5 + 2 + 1, 26 + 21 + 11 + 5 + 3 + 1, 26 + 21 + 11 + 6 + 1, 27 + 12 + 1,
27 + 12 + 2 + 1, 27 + 12 + 3 + 1, 27 + 12 + 4 + 1, 27 + 12 + 4 + 2 + 1, 27 + 12 + 4 + 3 + 1, 27 + 12 + 5 + 1, 27 + 12 + 5 + 2 + 1,
27 + 12 + 5 + 3 + 1, 27 + 12 + 6 + 1, 27 + 12 + 11 + 1, 27 + 12 + 11 + 2 + 1, 27 + 12 + 11 + 3 + 1, 27 + 12 + 11 + 4 + 1, 27 + 12 + 11 + 4 + 2 + 1,
27 + 12 + 11 + 4 + 3 + 1, 27 + 12 + 11 + 5 + 1, 27 + 12 + 11 + 5 + 2 + 1, 27 + 12 + 11 + 5 + 3 + 1, 27 + 12 + 11 + 6 + 1, 29 + 27 + 13 + 1,
29 + 27 + 13 + 2 + 1, 29 + 27 + 13 + 3 + 1, 29 + 27 + 13 + 4 + 1, 29 + 27 + 13 + 4 + 2 + 1, 29 + 27 + 13 + 4 + 3 + 1, 29 + 27 + 13 + 5 + 1,
29 + 27 + 13 + 5 + 2 + 1, 29 + 27 + 13 + 5 + 3 + 1, 29 + 27 + 13 + 6 + 1, 29 + 27 + 13 + 11 + 1, 29 + 27 + 13 + 11 + 2 + 1,
29 + 27 + 13 + 11 + 3 + 1, 29 + 27 + 13 + 11 + 4 + 1, 29 + 27 + 13 + 11 + 4 + 2 + 1, 29 + 27 + 13 + 11 + 4 + 3 + 1, 29 + 27 + 13 + 11 + 5 + 1,
29 + 27 + 13 + 11 + 5 + 2 + 1, 29 + 27 + 13 + 11 + 5 + 3 + 1, 29 + 27 + 13 + 11 + 6 + 1, 29 + 27 + 14 + 1, 29 + 27 + 14 + 2 + 1,
29 + 27 + 14 + 3 + 1, 29 + 27 + 14 + 4 + 1, 29 + 27 + 14 + 4 + 2 + 1, 29 + 27 + 14 + 4 + 3 + 1, 29 + 27 + 14 + 5 + 1, 29 + 27 + 14 + 5 + 2 + 1,
29 + 27 + 14 + 5 + 3 + 1, 29 + 27 + 14 + 6 + 1, 29 + 27 + 14 + 11 + 1, 29 + 27 + 14 + 11 + 2 + 1, 29 + 27 + 14 + 11 + 3 + 1,
29 + 27 + 14 + 11 + 4 + 1, 29 + 27 + 14 + 11 + 4 + 2 + 1, 29 + 27 + 14 + 11 + 4 + 3 + 1, 29 + 27 + 14 + 11 + 5 + 1, 29 + 27 + 14 + 11 + 5 + 2 + 1,
29 + 27 + 14 + 11 + 5 + 3 + 1, 29 + 27 + 14 + 11 + 6 + 1, 29 + 27 + 15 + 1, 29 + 27 + 15 + 2 + 1, 29 + 27 + 15 + 3 + 1, 29 + 27 + 15 + 4 + 1,
29 + 27 + 15 + 4 + 2 + 1, 29 + 27 + 15 + 4 + 3 + 1, 29 + 27 + 15 + 5 + 1, 29 + 27 + 15 + 5 + 2 + 1, 29 + 27 + 15 + 5 + 3 + 1, 29 + 27 + 15 + 6 + 1,
29 + 27 + 15 + 11 + 1, 29 + 27 + 15 + 11 + 2 + 1, 29 + 27 + 15 + 11 + 3 + 1, 29 + 27 + 15 + 11 + 4 + 1, 29 + 27 + 15 + 11 + 4 + 2 + 1,
29 + 27 + 15 + 11 + 4 + 3 + 1, 29 + 27 + 15 + 11 + 5 + 1, 29 + 27 + 15 + 11 + 5 + 2 + 1, 29 + 27 + 15 + 11 + 5 + 3 + 1, 29 + 27 + 15 + 11 + 6 + 1,
27 + 16 + 1, 27 + 16 + 2 + 1, 27 + 16 + 3 + 1, 27 + 16 + 4 + 1, 27 + 16 + 4 + 2 + 1, 27 + 16 + 4 + 3 + 1, 27 + 16 + 5 + 1, 27 + 16 + 5 + 2 + 1,
27 + 16 + 5 + 3 + 1, 27 + 16 + 6 + 1, 27 + 16 + 11 + 1, 27 + 16 + 11 + 2 + 1, 27 + 16 + 11 + 3 + 1, 27 + 16 + 11 + 4 + 1, 27 + 16 + 11 + 4 + 2 + 1,
27 + 16 + 11 + 4 + 3 + 1, 27 + 16 + 11 + 5 + 1, 27 + 16 + 11 + 5 + 2 + 1, 27 + 16 + 11 + 5 + 3 + 1, 27 + 16 + 11 + 6 + 1, 27 + 17 + 1,
27 + 17 + 2 + 1, 27 + 17 + 3 + 1, 27 + 17 + 4 + 1, 27 + 17 + 4 + 2 + 1, 27 + 17 + 4 + 3 + 1, 27 + 17 + 5 + 1, 27 + 17 + 5 + 2 + 1,
27 + 17 + 5 + 3 + 1, 27 + 17 + 6 + 1, 27 + 17 + 11 + 1, 27 + 17 + 11 + 2 + 1, 27 + 17 + 11 + 3 + 1, 27 + 17 + 11 + 4 + 1, 27 + 17 + 11 + 4 + 2 + 1,
27 + 17 + 11 + 4 + 3 + 1, 27 + 17 + 11 + 5 + 1, 27 + 17 + 11 + 5 + 2 + 1, 27 + 17 + 11 + 5 + 3 + 1, 27 + 17 + 11 + 6 + 1, 27 + 18 + 1,
27 + 18 + 2 + 1, 27 + 18 + 3 + 1, 27 + 18 + 4 + 1, 27 + 18 + 4 + 2 + 1, 27 + 18 + 4 + 3 + 1, 27 + 18 + 5 + 1, 27 + 18 + 5 + 2 + 1,
27 + 18 + 5 + 3 + 1, 27 + 18 + 6 + 1, 27 + 18 + 11 + 1, 27 + 18 + 11 + 2 + 1, 27 + 18 + 11 + 3 + 1, 27 + 18 + 11 + 4 + 1, 27 + 18 + 11 + 4 + 2 + 1,
27 + 18 + 11 + 4 + 3 + 1, 27 + 18 + 11 + 5 + 1, 27 + 18 + 11 + 5 + 2 + 1, 27 + 18 + 11 + 5 + 3 + 1, 27 + 18 + 11 + 6 + 1, 27 + 19 + 1,
27 + 19 + 2 + 1, 27 + 19 + 3 + 1, 27 + 19 + 4 + 1, 27 + 19 + 4 + 2 + 1, 27 + 19 + 4 + 3 + 1, 27 + 19 + 5 + 1, 27 + 19 + 5 + 2 + 1,
27 + 19 + 5 + 3 + 1, 27 + 19 + 6 + 1, 27 + 19 + 11 + 1, 27 + 19 + 11 + 2 + 1, 27 + 19 + 11 + 3 + 1, 27 + 19 + 11 + 4 + 1, 27 + 19 + 11 + 4 + 2 + 1,
27 + 19 + 11 + 4 + 3 + 1, 27 + 19 + 11 + 5 + 1, 27 + 19 + 11 + 5 + 2 + 1, 27 + 19 + 11 + 5 + 3 + 1, 27 + 19 + 11 + 6 + 1, 27 + 20 + 1,
27 + 20 + 2 + 1, 27 + 20 + 3 + 1, 27 + 20 + 4 + 1, 27 + 20 + 4 + 2 + 1, 27 + 20 + 4 + 3 + 1, 27 + 20 + 5 + 1, 27 + 20 + 5 + 2 + 1,
27 + 20 + 5 + 3 + 1, 27 + 20 + 6 + 1, 27 + 20 + 11 + 1, 27 + 20 + 11 + 2 + 1, 27 + 20 + 11 + 3 + 1, 27 + 20 + 11 + 4 + 1, 27 + 20 + 11 + 4 + 2 + 1,
27 + 20 + 11 + 4 + 3 + 1, 27 + 20 + 11 + 5 + 1, 27 + 20 + 11 + 5 + 2 + 1, 27 + 20 + 11 + 5 + 3 + 1, 27 + 20 + 11 + 6 + 1, 27 + 21 + 1,
27 + 21 + 2 + 1, 27 + 21 + 3 + 1, 27 + 21 + 4 + 1, 27 + 21 + 4 + 2 + 1, 27 + 21 + 4 + 3 + 1, 27 + 21 + 5 + 1, 27 + 21 + 5 + 2 + 1,
27 + 21 + 5 + 3 + 1, 27 + 21 + 6 + 1, 27 + 21 + 11 + 1, 27 + 21 + 11 + 2 + 1, 27 + 21 + 11 + 3 + 1, 27 + 21 + 11 + 4 + 1, 27 + 21 + 11 + 4 + 2 + 1,
27 + 21 + 11 + 4 + 3 + 1, 27 + 21 + 11 + 5 + 1, 27 + 21 + 11 + 5 + 2 + 1, 27 + 21 + 11 + 5 + 3 + 1, 27 + 21 + 11 + 6 + 1, 28 + 12 + 1,
28 + 12 + 2 + 1, 28 + 12 + 3 + 1, 28 + 12 + 4 + 1, 28 + 12 + 4 + 2 + 1, 28 + 12 + 4 + 3 + 1, 28 + 12 + 5 + 1, 28 + 12 + 5 + 2 + 1,
28 + 12 + 5 + 3 + 1, 28 + 12 + 6 + 1, 28 + 12 + 11 + 1, 28 + 12 + 11 + 2 + 1, 28 + 12 + 11 + 3 + 1, 28 + 12 + 11 + 4 + 1, 28 + 12 + 11 + 4 + 2 + 1,
28 + 12 + 11 + 4 + 3 + 1, 28 + 12 + 11 + 5 + 1, 28 + 12 + 11 + 5 + 2 + 1, 28 + 12 + 11 + 5 + 3 + 1, 28 + 12 + 11 + 6 + 1, 29 + 28 + 13 + 1,
29 + 28 + 13 + 2 + 1, 29 + 28 + 13 + 3 + 1, 29 + 28 + 13 + 4 + 1, 29 + 28 + 13 + 4 + 2 + 1, 29 + 28 + 13 + 4 + 3 + 1, 29 + 28 + 13 + 5 + 1,
29 + 28 + 13 + 5 + 2 + 1, 29 + 28 + 13 + 5 + 3 + 1, 29 + 28 + 13 + 6 + 1, 29 + 28 + 13 + 11 + 1, 29 + 28 + 13 + 11 + 2 + 1,
29 + 28 + 13 + 11 + 3 + 1, 29 + 28 + 13 + 11 + 4 + 1, 29 + 28 + 13 + 11 + 4 + 2 + 1, 29 + 28 + 13 + 11 + 4 + 3 + 1, 29 + 28 + 13 + 11 + 5 + 1,
29 + 28 + 13 + 11 + 5 + 2 + 1, 29 + 28 + 13 + 11 + 5 + 3 + 1, 29 + 28 + 13 + 11 + 6 + 1, 29 + 28 + 14 + 1, 29 + 28 + 14 + 2 + 1,
29 + 28 + 14 + 3 + 1, 29 + 28 + 14 + 4 + 1, 29 + 28 + 14 + 4 + 2 + 1, 29 + 28 + 14 + 4 + 3 + 1, 29 + 28 + 14 + 5 + 1, 29 + 28 + 14 + 5 + 2 + 1,
29 + 28 + 14 + 5 + 3 + 1, 29 + 28 + 14 + 6 + 1, 29 + 28 + 14 + 11 + 1, 29 + 28 + 14 + 11 + 2 + 1, 29 + 28 + 14 + 11 + 3 + 1,
29 + 28 + 14 + 11 + 4 + 1, 29 + 28 + 14 + 11 + 4 + 2 + 1, 29 + 28 + 14 + 11 + 4 + 3 + 1, 29 + 28 + 14 + 11 + 5 + 1, 29 + 28 + 14 + 11 + 5 + 2 + 1,
29 + 28 + 14 + 11 + 5 + 3 + 1, 29 + 28 + 14 + 11 + 6 + 1, 29 + 28 + 15 + 1, 29 + 28 + 15 + 2 + 1, 29 + 28 + 15 + 3 + 1, 29 + 28 + 15 + 4 + 1,
29 + 28 + 15 + 4 + 2 + 1, 29 + 28 + 15 + 4 + 3 + 1, 29 + 28 + 15 + 5 + 1, 29 + 28 + 15 + 5 + 2 + 1, 29 + 28 + 15 + 5 + 3 + 1, 29 + 28 + 15 + 6 + 1,
29 + 28 + 15 + 11 + 1, 29 + 28 + 15 + 11 + 2 + 1, 29 + 28 + 15 + 11 + 3 + 1, 29 + 28 + 15 + 11 + 4 + 1, 29 + 28 + 15 + 11 + 4 + 2 + 1,
29 + 28 + 15 + 11 + 4 + 3 + 1, 29 + 28 + 15 + 11 + 5 + 1, 29 + 28 + 15 + 11 + 5 + 2 + 1, 29 + 28 + 15 + 11 + 5 + 3 + 1, 29 + 28 + 15 + 11 + 6 + 1,
28 + 16 + 1, 28 + 16 + 2 + 1, 28 + 16 + 3 + 1, 28 + 16 + 4 + 1, 28 + 16 + 4 + 2 + 1, 28 + 16 + 4 + 3 + 1, 28 + 16 + 5 + 1, 28 + 16 + 5 + 2 + 1,
28 + 16 + 5 + 3 + 1, 28 + 16 + 6 + 1, 28 + 16 + 11 + 1, 28 + 16 + 11 + 2 + 1, 28 + 16 + 11 + 3 + 1, 28 + 16 + 11 + 4 + 1, 28 + 16 + 11 + 4 + 2 + 1,
28 + 16 + 11 + 4 + 3 + 1, 28 + 16 + 11 + 5 + 1, 28 + 16 + 11 + 5 + 2 + 1, 28 + 16 + 11 + 5 + 3 + 1, 28 + 16 + 11 + 6 + 1, 28 + 17 + 1,
28 + 17 + 2 + 1, 28 + 17 + 3 + 1, 28 + 17 + 4 + 1, 28 + 17 + 4 + 2 + 1, 28 + 17 + 4 + 3 + 1, 28 + 17 + 5 + 1, 28 + 17 + 5 + 2 + 1,
28 + 17 + 5 + 3 + 1, 28 + 17 + 6 + 1, 28 + 17 + 11 + 1, 28 + 17 + 11 + 2 + 1, 28 + 17 + 11 + 3 + 1, 28 + 17 + 11 + 4 + 1, 28 + 17 + 11 + 4 + 2 + 1,
28 + 17 + 11 + 4 + 3 + 1, 28 + 17 + 11 + 5 + 1, 28 + 17 + 11 + 5 + 2 + 1, 28 + 17 + 11 + 5 + 3 + 1, 28 + 17 + 11 + 6 + 1, 28 + 18 + 1,
28 + 18 + 2 + 1, 28 + 18 + 3 + 1, 28 + 18 + 4 + 1, 28 + 18 + 4 + 2 + 1, 28 + 18 + 4 + 3 + 1, 28 + 18 + 5 + 1, 28 + 18 + 5 + 2 + 1,
28 + 18 + 5 + 3 + 1, 28 + 18 + 6 + 1, 28 + 18 + 11 + 1, 28 + 18 + 11 + 2 + 1, 28 + 18 + 11 + 3 + 1, 28 + 18 + 11 + 4 + 1, 28 + 18 + 11 + 4 + 2 + 1,
28 + 18 + 11 + 4 + 3 + 1, 28 + 18 + 11 + 5 + 1, 28 + 18 + 11 + 5 + 2 + 1, 28 + 18 + 11 + 5 + 3 + 1, 28 + 18 + 11 + 6 + 1, 28 + 19 + 1,
28 + 19 + 2 + 1, 28 + 19 + 3 + 1, 28 + 19 + 4 + 1, 28 + 19 + 4 + 2 + 1, 28 + 19 + 4 + 3 + 1, 28 + 19 + 5 + 1, 28 + 19 + 5 + 2 + 1,
28 + 19 + 5 + 3 + 1, 28 + 19 + 6 + 1, 28 + 19 + 11 + 1, 28 + 19 + 11 + 2 + 1, 28 + 19 + 11 + 3 + 1, 28 + 19 + 11 + 4 + 1, 28 + 19 + 11 + 4 + 2 + 1,
28 + 19 + 11 + 4 + 3 + 1, 28 + 19 + 11 + 5 + 1, 28 + 19 + 11 + 5 + 2 + 1, 28 + 19 + 11 + 5 + 3 + 1, 28 + 19 + 11 + 6 + 1, 28 + 20 + 1,
28 + 20 + 2 + 1, 28 + 20 + 3 + 1, 28 + 20 + 4 + 1, 28 + 20 + 4 + 2 + 1, 28 + 20 + 4 + 3 + 1, 28 + 20 + 5 + 1, 28 + 20 + 5 + 2 + 1,
28 + 20 + 5 + 3 + 1, 28 + 20 + 6 + 1, 28 + 20 + 11 + 1, 28 + 20 + 11 + 2 + 1, 28 + 20 + 11 + 3 + 1, 28 + 20 + 11 + 4 + 1, 28 + 20 + 11 + 4 + 2 + 1,
28 + 20 + 11 + 4 + 3 + 1, 28 + 20 + 11 + 5 + 1, 28 + 20 + 11 + 5 + 2 + 1, 28 + 20 + 11 + 5 + 3 + 1, 28 + 20 + 11 + 6 + 1, 28 + 21 + 1,
28 + 21 + 2 + 1, 28 + 21 + 3 + 1, 28 + 21 + 4 + 1, 28 + 21 + 4 + 2 + 1, 28 + 21 + 4 + 3 + 1, 28 + 21 + 5 + 1, 28 + 21 + 5 + 2 + 1,
28 + 21 + 5 + 3 + 1, 28 + 21 + 6 + 1, 28 + 21 + 11 + 1, 28 + 21 + 11 + 2 + 1, 28 + 21 + 11 + 3 + 1, 28 + 21 + 11 + 4 + 1, 28 + 21 + 11 + 4 + 2 + 1,
28 + 21 + 11 + 4 + 3 + 1, 28 + 21 + 11 + 5 + 1, 28 + 21 + 11 + 5 + 2 + 1, 28 + 21 + 11 + 5 + 3 + 1, 28 + 21 + 11 + 6 + 1, 30 + 1, 30 + 2 + 1,
30 + 3 + 1, 30 + 4 + 1, 30 + 4 + 2 + 1, 30 + 4 + 3 + 1, 30 + 5 + 1, 30 + 5 + 2 + 1, 30 + 5 + 3 + 1, 30 + 6 + 1, 31 + 1, 31 + 2 + 1, 31 + 3 + 1,
31 + 4 + 1, 31 + 4 + 2 + 1, 31 + 4 + 3 + 1, 31 + 5 + 1, 31 + 5 + 2 + 1, 31 + 5 + 3 + 1, 31 + 6 + 1, 32, 35, and 36;

in the list above the numbers refer to the embodiments according to their numbering provided hereinabove whereas “+” indicates the dependency from another embodiment. The different individualized embodiments are separated by commas. In other words, “4+2+1” for example refers to embodiment 4) depending on embodiment 2), depending on embodiment 1), i.e. embodiment “4+2+1” corresponds to the compounds of embodiment 1) further limited by the features of the embodiments 2) and 4).

It is to be understood that the range of i as described in embodiments 33) and 34) shall be regarded as explicitly disclosed for each of the above-listed combinations.

Where the plural form is used for compounds, conjugates, salts, pharmaceutical compositions, diseases or the like, this is intended to mean also a single compound, conjugate, salt, pharmaceutical composition, disease or the like.

Any reference to a compound of Formula (I) as defined in any one of embodiments 1) to 36) is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. They may also be used for stabilisation in the form of buffers or lyophilized products including buffer. For reference see for example ‘Handbook of Pharmaceutical Salts. Properties, Selection and Use.’, P. Heinrich Stahl, Camille G. Wermuth (Eds.), Wiley-VCH, 2008 and ‘Pharmaceutical Salts and Co-crystals’, Johan Wouters and Luc Quéré (Eds.), RSC Publishing, 2012.

The present embodiments also include isotopically labelled, especially 2H (deuterium) labelled compounds of Formula (I), (Ia), (Ib), (Ic), (II), (IIa), (III) and (IIIa) which compounds are identical to the compounds of Formula (I), (Ia), (Ib), (Ic), (II), (IIa), (III) and (IIIa) except that one or more atoms have each been replaced by an atom having the same atomic number but an atomic mass different from the atomic mass usually found in nature. Isotopically labelled, especially ²H (deuterium) labelled compounds of Formula (I), (Ia), (Ib), (Ic), (II), (IIa), (III) and (IIIa), and salts thereof, are within the scope of the present embodiments. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment, the compounds of Formula (I), (Ia), (Ib), (Ic), (II), (IIa), (III) and (IIIa) are not isotopically labelled, or they are labelled only with one or more deuterium atoms. In a sub-embodiment, the compounds of Formula (I), (Ia), (Ib), (Ic), (II), (IIa), (III) and (IIIa) are not isotopically labelled at all. Isotopically labelled compounds of Formula (I), (Ia), (Ib), (Ic), (II), (IIa), (III) and (IIIa) may be prepared in analogy to the methods described hereinafter, but using the appropriate isotopic variation of suitable reagents or starting materials. For instance, the labelling may be performed within the linker L and/or spacer T.

The compounds of formula (I), (Ia), (Ib) and (Ic) as defined in any one of embodiments 1) to 36) and their pharmaceutically acceptable salts can be used as medicaments, e.g. in the form of pharmaceutical compositions for parenteral, enteral (such as oral) or nasal administration, in particular parenteral administration such am intramuscular, subcutaneous, and intradermal injections.

37) Hence, one aspect of the present invention relates to a pharmaceutical composition comprising, as active principle, an oligosaccharide-carrier protein conjugate according to any one of embodiments 1) to 36), in particular embodiments 35) and 36), or a pharmaceutically acceptable salt thereof, and at least one therapeutically inert excipient.

The production of the pharmaceutical compositions can be effected in a manner which will be familiar to any person skilled in the art (see for example Remington, The Science and Practice of Pharmacy, 23rd Edition (2021), published by Elsevier Inc., ISBN: 978-0-12-820007-0; Vaccine Development and Manufacturing, 1st edition (2014), published by John Wiley & Sons, ISBN: 9780470261941) by bringing the described compounds of Formula (I), (Ia), (Ib) and (Ic) or their pharmaceutically acceptable salts, optionally in combination with other therapeutically valuable substances, into a galenical administration form together with suitable, non-toxic, inert, therapeutically compatible solid or liquid carrier materials and, optionally, usual pharmaceutical adjuvants.

Said pharmaceutical composition is suitable for eliciting a protective immune response in a human and/or animal (especially a mammal (including a human)) host, and therefore is useful for the prevention and/or treatment of diseases associated with Klebsiella pneumoniae bacteria. Preferably, said pharmaceutical composition is suitable for use in human.

The terms “prevention”, “preventing” and/or “prophylaxis” are used synonymously and refer to inhibiting the initial onset of a pathologic process, such that the pathologic process that could eventually lead to development of symptoms never develops or that symptoms develop in lower, non-dangerous intensity (i.e. preventing the development of a disease, disorder, or condition in a prophylactic manner).

The present pharmaceutical composition is suitable for administration to animal (and, in particular, human) patients, and thus include both human and veterinary uses. It may be used in a method of raising an immune response in a patient, comprising the step of administering the composition to the patient.

The pharmaceutical compositions of the present invention may be administered before a subject is exposed to Klebsiella pneumoniae and/or after a subject is exposed to a Klebsiella pneumoniae. Preferably, it is used before a subject is exposed to Klebsiella pneumoniae.

Pharmaceutical compositions are preferably in aqueous form, particularly at the point of administration, but they can also be presented in non-aqueous liquid forms or in dried forms e.g. as gelatin capsules, or as lyophilisates, etc., Solid powders that are obtained e.g. by spray drying, spray-freeze drying, vacuum or air-drying, or lyophilisation, may be reconstituted before use.

The pharmaceutical composition may comprise one or more therapeutically inert excipients. Such excipient may be selected from the group consisting of citric acid monohydrate, sodium citrate, sodium citrate dihydrate, acetic acid, sodium hydroxide, tromethamine, tromethamine hydrochloride (to adjust pH), cholesterol, sorbitan trioleate, DSPC (1,2-distearoyl-sn-glycero-3-phosphocholine), and (4-hydroxybutyl) azanediyl)bis(hexane-6,1-diyl)bis(2-hexyldecanoate), polydimethylsiloxane (antifoam), ascorbic acid (antioxidant).

The excipient may serve to adjust tonicity, such as sodium chloride (NaCl), which may be present at from 1 to 20 mg/ml. Other salts that may be present include potassium chloride, potassium dihydrogen phosphate, disodium phosphate dehydrate, magnesium chloride, calcium chloride, etc.

The pharmaceutical composition may include one or more excipients which serve as preservatives which may be selected from the group consisting of 2-phenoxyethanol, benzethonium chloride, EDTA (ethylenediaminetetraacetic acid), formaldehyde, phenol and thiomersal (thimerosal). Mercury-free compositions are preferred, and preservative-free vaccines can be prepared.

The pharmaceutical composition may include one or more excipients which serve as surfactants which may be selected from the group consisting of polysorbate 20 (polyoxyethylene (20) sorbitan monolaurate), polysorbate 80 (polyoxyethylene (80) sorbitan monooleate), nonylphenol ethoxylate, octoxynol-10 and sodium deoxycholate.

The pharmaceutical composition may include compounds (with or without an insoluble metal salt) in plain water (e.g. water for injection, w.f.i.), but will usually include one or more buffers. Typical buffers include: a phosphate buffer; a Tris buffer; a borate buffer; a succinate buffer; a histidine buffer (particularly with an aluminum hydroxide adjuvant); or a citrate buffer. Buffer salts will typically be included in the 5-20 mM range.

Pharmaceutical compositions typically have a pH between 5.0 and 9.5 e.g. between 6.0 and 8.0.

The pharmaceutical composition may further include one or more stabilizer(s).

Pharmaceutical compositions are preferably sterile and gluten free.

38) A further embodiment of the present invention relates to the pharmaceutical composition according to embodiment 37), further comprising an adjuvant.

The term “adjuvant” as used herein refers to an immunological adjuvant i.e. a material used in a vaccine composition that modifies or augments the effects of said vaccine by enhancing the immune response to a given antigen contained in the vaccine without being antigenically related to it. For the person skilled in the art, classically recognized examples of immunological adjuvants include, but are not restricted to aluminum or calcium salt based adjuvants, saponins or saponin-based adjuvants (e.g. Matrix-M), CpG oligodexynucleotide based adjuvants (e.g. CpG 1018), oil-in-water emulsions (e.g. Freund's adjuvant, MF59), activators of natural killer T cells (NKT cells) or invariant NKT cells (e.g., glycosphingolipids such as KRN7000), toll-like receptor 1/2 (TLR-1/2) agonists (e.g., Pam3CSK4), TLR-3 agonists (e.g., Poly(I:C)), TLR-4 agonists (e.g., lipopolysaccharide), TLR-5 agonists (e.g., flagellin), TLR-7/8 agonists (e.g., resiquimod), immunomodulatory proteins (e.g., detoxified heat-labile enterotoxin (dmLT) from Escherichia coli), TLR-4 agonist glucopyranosyl lipid adjuvant-stable emulsion (GLA-SE) and monophosphoryl lipid A (MPL), non-ionic block polymers, cytokines (e.g., type 1 interferon (IFN), granulocyte-macrophage colony-stimulating factor (GM-CSF), interleukins), papain-like cysteine proteases, and many others such as e.g. AS04, AS03, AS01B, and formulations of the above mentioned adjuvants as liposomes or nanoparticles prepared with lipids such as DOPC (1,2-dioleoyl-sn-glycero-3-phosphocholine), DSPC (1,2-distrearoyl-sn-glycero-3-phosphocholine), cholesterol and/or ALC-0315, formulations as virus-like particles, and co-formulations of the abovementioned adjuvants, especially co-formulation including aluminum or calcium salt based adjuvants.

The adjuvant “aluminum”, “aluminum-based adjuvant” or “aluminum salt-based adjuvant” is one or more of the following: amorphous aluminum hydroxyphosphate sulfate (AAHS), aluminum hydroxide, aluminum phosphate, and potassium aluminum sulfate (Alum).

An example for a calcium-based or calcium salt-based adjuvant is calcium phosphate.

Matrix-M is a saponin-based adjuvant composed of nanoparticles from saponins extracted from Quillaja saponaria (soapbark) trees, cholesterol, and phospholipids.

CpG based adjuvants are immunostimulatory oligodeoxynucleotides bearing one or more CpG motifs (CpG ODN) that are unmethylated cytosine-guanine dinucleotides. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9).

Freund's adjuvant is an oil-in-water adjuvant based on mineral oil.

MF59 is an oil-in-water emulsion comprising 4.3% w/v squalene, 0.5% w/v polysorbate 80 (Tween 80), and 0.5% w/v sorbitan trioleate (Span 85).

Glycosphingolipids are a class of lipids that stimulate unconventional invariant T-cell receptors on NKT cells or iNKT cells, when the glycosphingolipid is presented MHC class I-related molecules such as CD1d.

Pam3CSK4 (Pam3CysSerLys4) is a synthetic triacylated lipopeptide that is a ligand for TLR-1 and TLR-2. It mimics the acylated amino terminus of bacterial lipopeptides.

Poly(I:C) is a polymer and analogue of double-stranded RNA, consisting of one strand of a polymer of inosinic acid and one strand of a polymer of cytidylic acid. It stimulates TLR-3 and simulates viral infections.

Lipopolysaccharide (LPS) is a membrane component of Gram-negative bacteria and a stimulator of TLR-4.

Flagellin is a globular protein that forms the filaments of bacterial flagella. Flagellin activates TLR-5 and TLR-11.

Resiquimod (R848; 1-[4-Amino-2-(ethoxymethyl)-1H-imidazo[4,5-c]chinolin-1-yl]-2-methylpropan-2-ol) is an immune response modifier and small molecule that activates TLR-7 and TLR-8.

dmLT is the double-mutant (thereby detoxified) of heat-labile enterotoxin from Escherichia coli. It is an effective mucosal and systemic adjuvant.

GLA-SE is an oil-in-water emulsion adjuvant that is prepared by combining aqueous glucopyranosyl lipid A (GLA), a TLR-4 agonist, with squalene.

MPL (monophosphoryl lipid A), a truncated LPS, is a clinically used TLR-4 agonist.

Nonionic Block Polymers (NBPs) suitable as adjuvants are simple copolymers of polyoxyethylene (POE) and hydrophobic polyoxypropylene (POP) and differ in molecular weight, percentage POE and the mode of linkage of POE and POP-groups.

Cytokines are small proteins secreted by cells that affect the interaction and communication between cells. Typically, cytokines activate the target cell, leading to the secretion of additional cytokines and signaling cascades. Cytokines are involved in the induction of innate and adaptive immunity. As adjuvants, cytokines can be used as recombinant proteins or can be encoded on DNA molecules such as plasmids.

Papain-like cysteine proteases are derived from viruses, bacteria, yeast, protozoa, plants or animals and contain a cysteine thiol at the active site. This class of proteases can stimulate Th2 type immune responses.

AS04 (Adjuvant System 04) is a complex of MPL (3-O-desacyl-4′-monophosphoryl lipid A) and aluminum hydroxide or aluminum phosphate.

AS03 (Adjuvant System 03) is a squalene-in-water emulsion with DL-alpha-tocopherol (vitamin E) and polysorbate 80.

AS01B is a mixture of 3-O-desacyl-4′-monophosphoryl lipid A (MPL) and the saponin QS-21.

Preferred adjuvants are aluminum-based adjuvants, in particular aluminum hydroxide.

39) A further aspect of the present invention relates to the oligosaccharide-carrier protein conjugate according to any one of embodiments 1) to 36), in particular embodiments 35) and 36), or a pharmaceutically acceptable salt thereof, for the use as a medicament, in particular as a vaccine. In other words, the invention relates to a vaccine comprising the oligosaccharide-carrier protein conjugate according to any one of embodiments 1) to 36), in particular embodiments 35) and 36), or a pharmaceutically acceptable salt thereof. Preferably, the vaccine is used for active vaccination.

40) A further aspect of the present invention relates to an oligosaccharide-carrier protein conjugate according to any one of embodiments 1) to 36), in particular embodiments 35) and 36), or a pharmaceutically acceptable salt thereof, for the use in the prevention and/or treatment of a K. pneumoniae infection.

41) A further embodiment of the present invention relates to an oligosaccharide-carrier protein conjugate according to any one of embodiments 1) to 36), in particular embodiments 35) and 36), or a pharmaceutically acceptable salt thereof, for the use in the prevention and/or treatment of K. pneumoniae infections in individuals of 50 years or older; hospital acquired (i.e. nosocomial) K. pneumoniae infections, for instance nosocomial pneumonia, nosocomial bloodstream infections and nosocomial urinary tract infections; community-acquired K. pneumoniae infections; as well as pneumonia, bronchitis, meningitis, urinary tract infection, intra-abdominal infections, wound infection, infection of blood, osteomyelitis, bacteremia, septicemia, liver abscess, and inflammatory bowel disease (IBD) all caused by K. pneumoniae infection.

A population-based strategy for vaccination of individuals of 50 years or older against K. pneumoniae infections is desirable, because this population is particularly susceptible to K. pneumoniae infections, in particular individuals of 60 years or older and at risk of exposure to K. pneumoniae and/or anticipated weakened immune system.

K. pneumoniae is a notorious pathogen frequently responsible for hospital acquired (i.e. nosocomial) respiratory and urinary tract infections. It is the second most common cause of Gram-negative bacteremia. Drug resistant isolates are associated with high mortality (greater than 50% according to some studies), add significantly to hospital stays, and are especially problematic in ICUs.

Therefore, it is desired to prevent hospital acquired (i.e. nosocomial) K. pneumoniae infections, in particular in populations at high risk of exposure, including patients who will undergo elective surgery with hospital stays longer than 72 hours (e.g., joint replacements), patients with weakened immune systems and patients who anticipate having weakened immune systems (e.g., those on solid organ transplant wait lists, non-urgent solid tumor surgery followed by chemotherapy). In these populations, vaccination 2-8 weeks prior to surgery, optionally followed by a booster may be applicable.

Moreover, the prevention of community-acquired infections in specific target groups such as healthcare workers or elderly (60 years or older) in long-term care facilities or nursing homes is desired. The term “community-acquired K. pneumoniae infections” relates to any K. pneumoniae infection acquired in the community. In contrast to a nosocomial (hospital-acquired) infection.

Furthermore, the present oligosaccharide-carrier protein conjugate according to any one of embodiments 1) to 36), in particular embodiments 35) and 36), or a pharmaceutically acceptable salt thereof, may be used in the prevention and/or treatment of pneumonia, bronchitis, meningitis, urinary tract infection, intra-abdominal infections, wound infection, infection of blood, osteomyelitis, bacteremia, septicemia, liver abscess, and inflammatory bowel disease (IBD), all caused by K. pneumoniae infection.

42) A further embodiment of the present invention relates to an oligosaccharide-carrier protein conjugate according to any one of embodiments 1) to 36), in particular embodiments 35) and 36), or a pharmaceutically acceptable salt thereof, for the use in the prevention and/or treatment of the K. pneumoniae infections as listed in embodiments 40) and 41) above, wherein K. pneumoniae is selected from O-serotypes comprising 01.

43) For any avoidance of doubt, oligosaccharide-carrier protein conjugate according to any one of embodiments 1) to 36), in particular embodiments 35) and 36), or a pharmaceutically acceptable salt thereof, as well as the pharmaceutical composition of embodiment 37) or 38), and the vaccine according to embodiment 39) are likewise suitable for the prevention and/or the treatment of the K. pneumoniae infections as listed in any one of embodiments 40), 41) and 42).

44) Preferably, the oligosaccharide-carrier protein conjugate according to any one of embodiments 1) to 36), in particular embodiments 35) and 36), or a pharmaceutically acceptable salt thereof, as well as the pharmaceutical composition of embodiment 37) or 38), and the vaccine according to embodiment 39) are suitable for the prevention or prophylaxis of the K. pneumoniae infections as listed in any one of embodiments 40), 41) and 42).

45) A further aspect of the present invention relates to a method of eliciting an immune response against K. pneumoniae in a human and/or animal (especially a mammal (including a human)) host, comprising administering to the human and/or animal an effective amount of the oligosaccharide-carrier protein conjugate according to any one of embodiments 1) to 36), in particular embodiments 35) and 36), or a pharmaceutically acceptable salt thereof. The administered amount is preferably from 0.05 μg to 30 μg glycan per immunization of the human patient. The term “glycan” refers to antigen, i.e. oligosaccharide excluding linker L and spacer T. Possibly, more than one immunization is required.

46) Likewise, an embodiment of the present invention relates to a method of eliciting an immune response against K. pneumoniae in a human and/or animal (especially a mammal (including a human)) host, comprising administering to the human and/or animal an effective amount of the composition according to embodiment 37) or 38), as well as the vaccine according to embodiment 39).

47) For avoidance of any doubt, if oligosaccharide-carrier protein conjugates according to any one of embodiments 1) to 36), in particular embodiments 35) and 36), or pharmaceutically acceptable salts thereof, are described as useful for the prevention and/or treatment of a K. pneumoniae infection according to any one of embodiments 40), 41) and 42), such oligosaccharide-carrier protein conjugates are likewise suitable for use in the preparation of a medicament for the prevention and/or treatment of said K. pneumoniae infection according to any one of embodiments 40), 41) and 42).

48) A further aspect of the present invention relates to a multivalent vaccine comprising the oligosaccharide-carrier protein conjugate according to any one of embodiments 1) to 36), preferably the oligosaccharide-carrier protein conjugate according embodiments 35) or 36), or a pharmaceutically acceptable salt thereof.

The term “multivalent vaccine” in this respect relates to a vaccine comprising antigens against two or more different K. pneumoniae strains, in particular to two or more pathogenic K. pneumoniae strains.

49) A further aspect of the present invention relates to an intermediate compound for preparing the oligosaccharide-carrier protein conjugate according to any one of embodiments 12) to 36), having the formula (II)

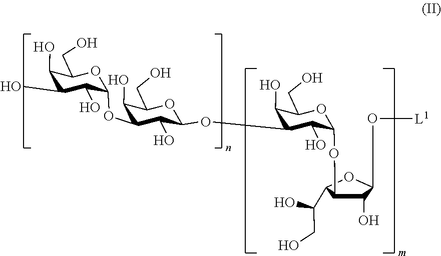

wherein
m is 4, 5 or 6, preferably 4 or 5, more preferably 4;
n is 5, 6 or 7, preferably 6 or 7, more preferably 6;
L¹ represents
   *—(C₂₋₁₀)alkylene-NH₂;
   *—(CH₂CH₂O)ₐ—CH₂CH₂NH₂, wherein a is 1, 2 or 3;
   *—CH₂CH₂S—CH₂CH₂NH₂;
   *—(C₂₋₁₀)fluoroalkylene-NH₂;
   *—(CH₂)_{c}NHC(O)(CH₂)_{d}—NH₂, wherein c and d are independently from each other from 2 to 6;
   *—(CH₂)ₑNHC(O)NH(CH₂)ₕ—NH₂, wherein e and h are independently from each other from 2 to 6;
   *—(C₁₋₁₀)alkylene-C(O)—NH—(C₂₋₁₀)alkylene-NH₂;
   *—(C₂₋₁₀)alkylene-O—NH₂; or
   —(C₂₋₁₀)alkylene-SH;
or a pharmaceutically acceptable salt thereof.

50) A further embodiment relates to the intermediate compound according to embodiment 49), or a pharmaceutically acceptable salt thereof, wherein m is 4 and n is 6.

51) A further embodiment relates to the intermediate compound according to embodiment 49) or 50), or a pharmaceutically accepable salt thereof, wherein
L¹ represents
   *—(CH₂)ₗ—NH₂; wherein l is from 2 to 10;
   *—(CH₂CH₂O)ₐ—CH₂CH₂NH₂, wherein a is 1, 2 or 3;
   *—CH₂CH₂S—CH₂CH₂NH₂;
   *—(C₂₋₁₀)fluoroalkylene-NH₂ with fluoroalkylene being a saturated straight chain;
   *—(CH₂)_{c}NHC(O)(CH₂)_{d}—NH₂, wherein c and d are independently from each other from 2 to 6;
   *—(CH₂)ₑNHC(O)NH(CH₂)ₕ—NH₂, wherein e and h are independently from each other from 2 to 6;
   *—(CH₂)_{g}—O—NH₂, wherein g is from 2 to 10;
   *—(CH₂)ᵤ—C(O)—NH—(CH₂)_{u′}—NH₂; wherein u is from 1 to 10 and u′ is from 2 to 10; or
   *—(CH₂)_{q}S—H, wherein q is from 2 to 10.

It is to be understood that in an analogous manner, embodiments 14) to 21) disclose further preferred L¹ which bear terminal amino- or SH-groups as demonstrated in embodiment 49).

52) A further embodiment is the intermediate compound of formula (IIa) with the following structure:

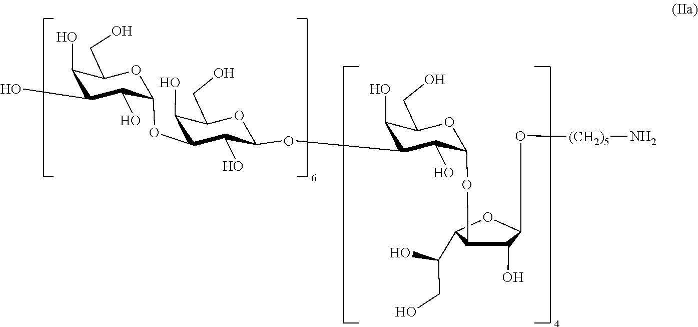

or a pharmaceutically acceptable salt thereof.

53) A further aspect of the present invention relates to an intermediate compound for preparing the oligosaccharide-carrier protein conjugate according to any one of embodiments 12) to 36), having the formula (III):

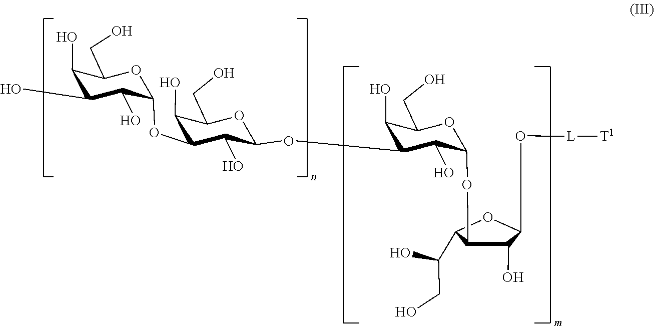

wherein
m is 4, 5 or 6, preferably m is 4 or 5, most preferably 4;
n is 5, 6 or 7, preferably n is 6 or 7, most preferably 6;
L represents
   *—(C₂₋₁₀)alkylene-NH—;
   *—(CH₂CH₂O)ₐ—CH₂CH₂NH—, wherein a is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{d}—NH—, wherein c and d are independently from each other from 2 to 6;
   *—(CH₂)ₑNHC(O)NH(CH₂)ₕ—NH—, wherein e and h are independently from each other from 2 to 6;
   *—(C₁₋₁₀)alkylene-C(O)—NH—(C₂₋₁₀)alkylene-NH—; or
   —(C₂₋₁₀)alkylene-O—NH—; and
T¹ represents
   —C(O)—(C₀₋₁₀)alkylene-C(O)X;
   —C(O)—CH₂CH₂—(OCH₂CH₂)ᵣ—C(O)X, wherein r is from 1 to 5;
   —C(O)—CH₂ (CH₂)_{f}—(SCH₂ (CH₂)_{f′})_{f″}—C(O)X, wherein f is 0 or 1, f′ is 0 or 1, and f″ is 1, 2, or 3;

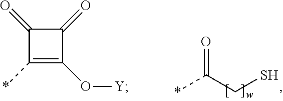

wherein w is 1 or 2; or

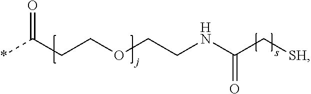

wherein j is from 1 to 4, preferably 1, and s is 1 or 2;
—C(O)X represents —C(O)OH or an activated ester;
Y represents Me, Et, Bu or —(CH₂CH₂O)₃CH₃,
or a pharmaceutically acceptable salt thereof.

Preferably, X represents

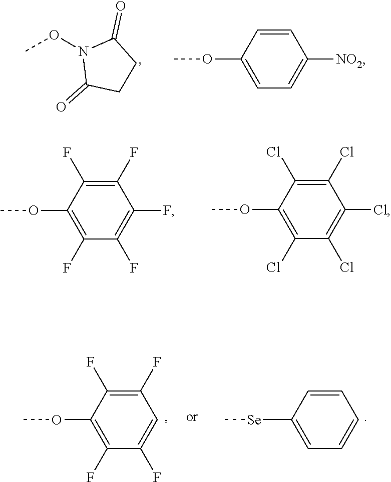

The term “activated ester” refers to a functionalized carboxylic acid with enhanced reactivity toward amines (in comparison to a carboxylic acid), for the reaction with the amino group of a lysine residue of CRM₁₉₇.

It is to be understood that embodiments 13) to 21) disclose further preferred L which are encompassed in the present embodiment. Moreover, it is to be understood that in an analogous manner, embodiments 22) to 28) disclose further preferred T¹ which bear terminal X-, OY-, or SH-groups as demonstrated in the present embodiment. I.e. -T- as disclosed in these embodiments bear terminal X-, OY-, or SH-groups for coupling to amino groups of CRM₁₉₇. For the avoidance of any doubt, the terminal “C(O)—” as disclosed in T in these embodiments translate to T¹ with “C(O)X”, and in case of squaric acid, the attachment point to CRM₁₉₇ is denoted as “O—Y”, and R¹ is H. These preferred T¹ are to be regarded as explicitly disclosed.

54) A further embodiment is the intermediate compound of formula (Illa), or a pharmaceutically acceptable salt thereof, with the following structure:

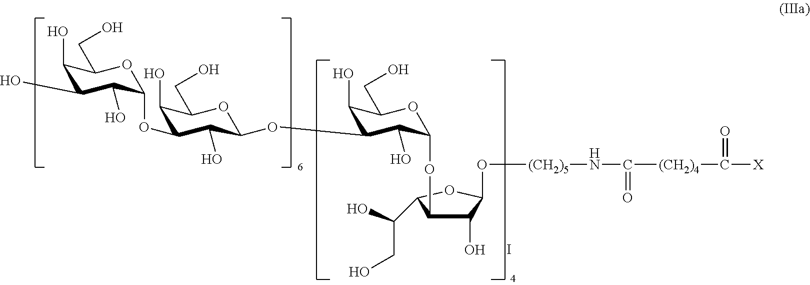

wherein-C(O)X represents-C(O)OH or an activated ester, wherein preferably
   X represents

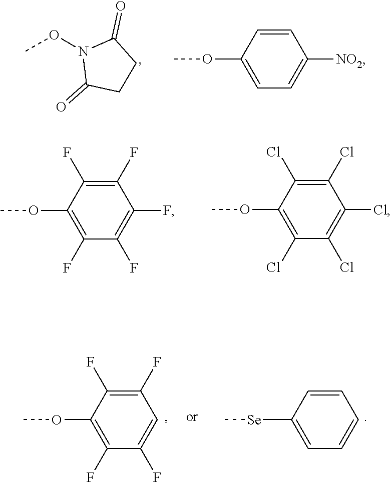

55) A further aspect of the present invention relates to an assay comprising the compound of formula (IV)

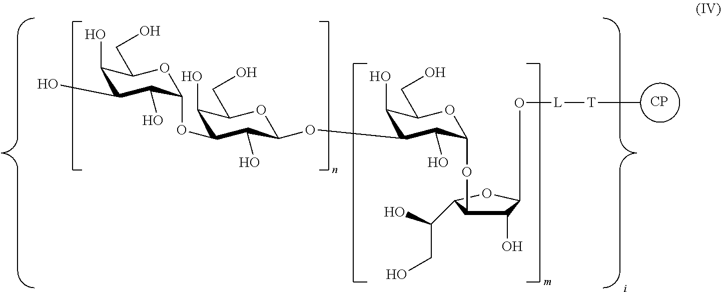

wherein m, n, i, L and T are as described in any one of embodiments 1) to 36), in particular embodiments 35) and 36), and CP is a carrier protein.

In this embodiment, the carrier protein CP may be any carrier protein suitable for assays, in particular ELISA. A preferred carrier protein is BSA.

The synthesis of a compound of the antigen of formula (IV) conjugated to BSA is described and exemplified in the experimental part. It is to be understood that this synthesis applies analogously to all antigens of formula (IV) so that the skilled person can prepare them.

The assay of this embodiment is suitable for the detection of antibodies against K. pneumoniae O1 strains.

56) A further aspect of the present invention relates to the oligosaccharide-carrier protein conjugate according to any one of embodiments 12) to 36), or a pharmaceutically acceptable salt thereof, wherein the conjugate is obtainable by or prepared by conjugating the compound of formula (III):

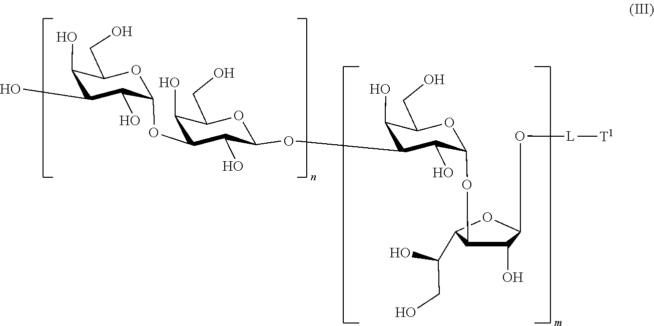

wherein
m is 4, 5 or 6, preferably m is 4 or 5, most preferably 4;
n is 5, 6 or 7, preferably n is 6 or 7, most preferably 6;
L represents
   *—(C₂₋₁₀)alkylene-NH—;
   *—(CH₂CH₂O)ₐ—CH₂CH₂NH—, wherein a is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{d}—NH—, wherein c and d are independently from each other from 2 to 6;
   *—(CH₂)ₑNHC(O)NH(CH₂)ₕ—NH—, wherein e and h are independently from each other from 2 to 6;
   *—(C₁₋₁₀)alkylene-C(O)—NH—(C₂₋₁₀)alkylene-NH—; or
   *—(C₂₋₁₀)alkylene-O—NH—; and
a) T¹ represents
   —C(O)—(C₀₋₁₀)alkylene-C(O)X;
   —C(O)—CH₂CH₂—(OCH₂CH₂)—C(O)X, wherein r is from 1 to 5;
   —C(O)—CH₂(CH₂)_{f}—(SCH₂(CH₂)_{f′})_{f″}—C(O)X, wherein f is 0 or 1, f′ is 0 or 1, and f″ is 1, 2, or 3;

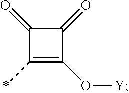

—C(O)X represents —C(O)OH or an activated ester,
Y represents Me, Et, Bu or —(CH₂CH₂O)₃CH₃;
to a lysine residue of CRM₁₈₇; or
b) conjugating the compound of formula (III) wherein T¹ represents

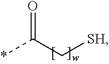

wherein w is 1 or 2; or

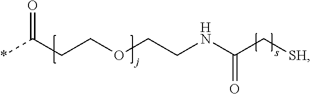

wherein j is from 1 to 4, preferably 1, and s is 1 or 2; or
wherein L-T¹ represents
   *—(C₂₋₁₀)alkylene-SH;
to modified CRM₁₉₇ selected from the group consisting of:

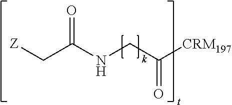

wherein Z is Br or I, k is 2 or 3, and t is from 1 to 28;

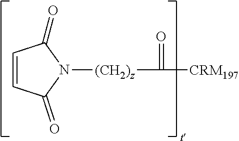

wherein z is 2 or 3, and t′ is from 1 to 28; or

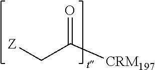

wherein Z is Br or I, and t″ is from 1 to 28.

Preferably, X represents

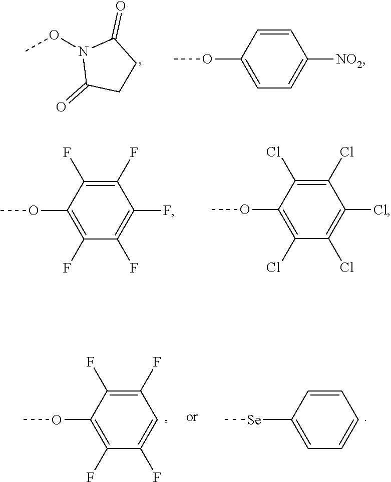

It is to be understood that embodiments 13) to 21) disclose further preferred L which are encompassed in the present embodiment. Moreover, it is to be understood that in an analogous manner, embodiments 22) to 28) disclose further preferred T¹ which bear terminal X-, OY-, or SH-groups as demonstrated in the present embodiment. I.e. -T- as disclosed in these embodiments bear terminal X-, OY-, or SH-groups for coupling to amino groups of CRM₁₉₇. For the avoidance of any doubt, the terminal “C(O)—” as disclosed in T in these embodiments translate to T¹ with “C(O)X”, and in case of squaric acid, the attachment point to CRM₁₉₇ is denoted as “O—Y”, and R¹ is H. These preferred T¹ are to be regarded as explicitly disclosed.

57) A further aspect of the present invention relates to a process for preparing the oligosaccharide-carrier protein conjugate according to any one of embodiments 12) to 36), or a pharmaceutically acceptable salt thereof, wherein the process comprises conjugating the compound of formula (III):

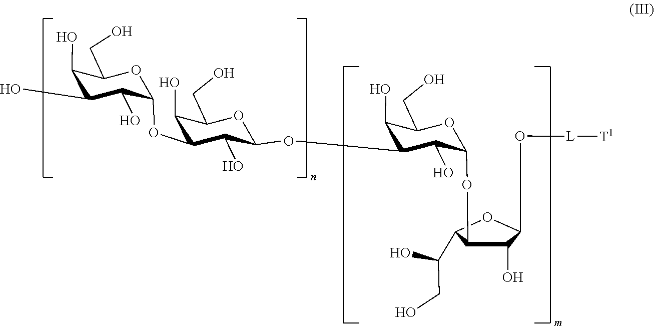

wherein
m is 4, 5 or 6, preferably m is 4 or 5, most preferably 4;
n is 5, 6 or 7, preferably n is 6 or 7, most preferably 6;
L represents
   *—(C₂₋₁₀)alkylene-NH—;
   *—(CH₂CH₂O)ₐ—CH₂CH₂NH—, wherein a is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{d}—NH—, wherein c and d are independently from each other from 2 to 6;
   *—(CH₂)ₑNHC(O)NH(CH₂)ₕ—NH—, wherein e and h are independently from each other from 2 to 6;
   *—(C₁₋₁₀)alkylene-C(O)—NH—(C₂₋₁₀)alkylene-NH—; or
   *—(C₂₋₁₀)alkylene-O—NH—; and
a) T¹ represents
   —C(O)—(C₀₋₁₀)alkylene-C(O)X;
   —C(O)—CH₂CH₂—(OCH₂CH₂)ᵣ—C(O)X, wherein r is from 1 to 5;
   —C(O)—CH₂(CH₂)_{f}—(SCH₂ (CH₂)_{f′})_{f″}—C(O)X, wherein f is 0 or 1, f′ is 0 or 1, and f″ is 1, 2, or 3;

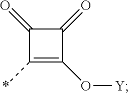

—C(O)X represents —C(O)OH or an activated ester,
Y represents Me, Et, Bu or —(CH₂CH₂O)₃CH₃;
to a lysine residue of CRM₁₉₇; or
b) conjugating the compound of formula (III) wherein T¹ represents

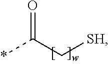

wherein w is 1 or 2; or

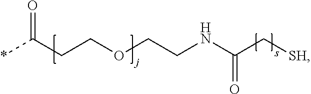

wherein j is from 1 to 4, preferably 1, and s is 1 or 2; or
wherein L-T¹ represents
   *—(C₂₋₁₀)alkylene-SH;
to modified CRM₁₉₇ selected from the group consisting of:

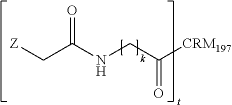

wherein Z is Br or I, k is 2 or 3, and t is from 1 to 28;

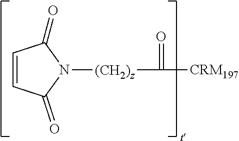

wherein z is 2 or 3, and t′ is from 1 to 28; or

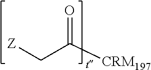

wherein Z is Br or I, and t″ is from 1 to 28.

Preferably, X represents

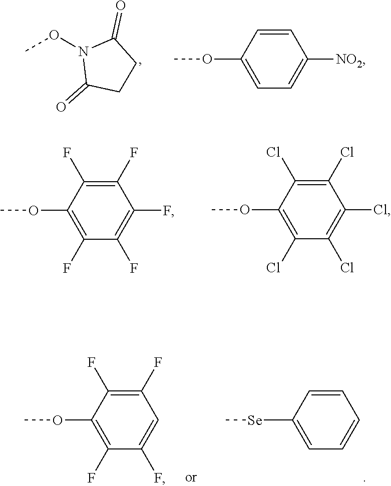

It is to be understood that embodiments 13) to 21) disclose further preferred L which are encompassed in the present embodiment. Moreover, it is to be understood that in an analogous manner, embodiments 22) to 28) disclose further preferred T¹ which bear terminal X-, OY-, or SH-groups as demonstrated in the present embodiment. I.e. -T- as disclosed in these embodiments bear terminal X-, OY-, or SH-groups for coupling to amino groups of CRM₁₉₇. For the avoidance of any doubt, the terminal “C(O)—” as disclosed in T in these embodiments translate to T¹ with “C(O)X”, and in case of squaric acid, the attachment point to CRM₁₉₇ is denoted as “O—Y”, and R¹ is H. These preferred T¹ are to be regarded as explicitly disclosed.

Whenever the word “between” or “to” is used to describe a numerical range, it is to be understood that the end points of the indicated range are explicitly disclosed and included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

However, for the avoidance of any doubt, the definition “a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇” means that the oxygen at C1 and the nitrogen of the amino group of the lysine at the CRM₁₉₇ do not count to the numbering of the so-defined backbone.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of about 25° C.

### Preparation of Compounds of Formula (I), (Ia), (Ib), (Ic), (II), (IIa), (IIb), (III), (IIIa) and (IV)

A further aspect of the invention is a process for the preparation of compounds of Formula (I), (Ia), (Ib), (Ic), (II), (IIa), (IIb), (III), (IIIa) and (IV). Compounds according to Formula (I), (Ia), (Ib), (Ic), (II), (IIa), (IIb), (III), (Illa) and (IV) of the present invention can be prepared from commercially available or well-known starting materials according to the methods described in the experimental part; by analogous methods; or according to the general sequence of reactions outlined below, wherein L, T, L¹, T¹, X and Y are as defined for Formula (I), (Ia), (Ib), (Ic), (II), (IIa), (IIb), (III), (IIIa) and (IV). Other abbreviations used herein are explicitly defined, or are as defined in the experimental section.

The synthesis of the compounds of the present invention requires protection group strategy. Though such protecting group strategy may be sophisticated, the use of protecting groups is well known in the art (see for example “Protective Groups in Organic Synthesis”, T. W. Greene, P. G. M. Wuts, Wiley-Interscience, 1999). The compounds obtained may also be converted into salts, especially pharmaceutically acceptable salts thereof in a manner known per se.

### General Preparation Routes:

### Antigen Representation

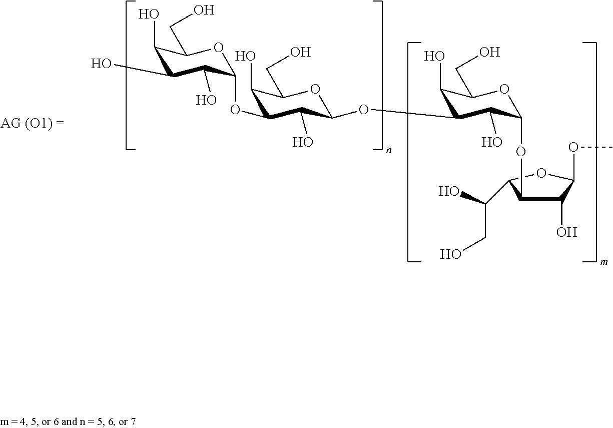

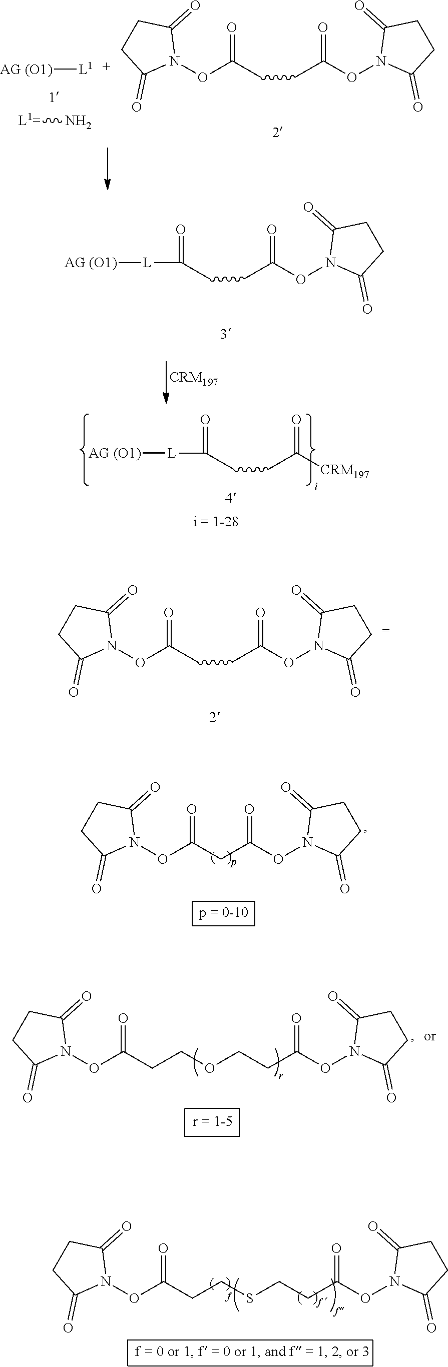

O₁-Antigen 1′ in appropriate solvent (e.g., DMSO) in a vial at rt is treated with activated Bis-NHS ester of the diacid 2′ (e.g., Bis-NHS adipate, which is commercially available or can be prepared by the person skilled in the art using corresponding Bis-acid and N-hydroxy succinic acid) (Odom, O. W., Biochemistry, Vol. 29, No. 48, 1990) (5-20 equiv.) in DMSO in presence of triethylamine and stirred for 3 h at rt. The Antigen-NHS ester 3′ is precipitated out by adding EtOAc, and centrifuged, subsequently the precipitate is washed with EtOAc, dried in vacuum before taken for the next step. The buffer solution containing Antigen-NHS ester 3′ (25-100 equiv.) and CRM₁₉₇ is stirred at rt for 20-24 h. The resulting O₁-Antigen-CRM₁₉₇ conjugate 4′ is washed, purified and stored using appropriate buffer solution.

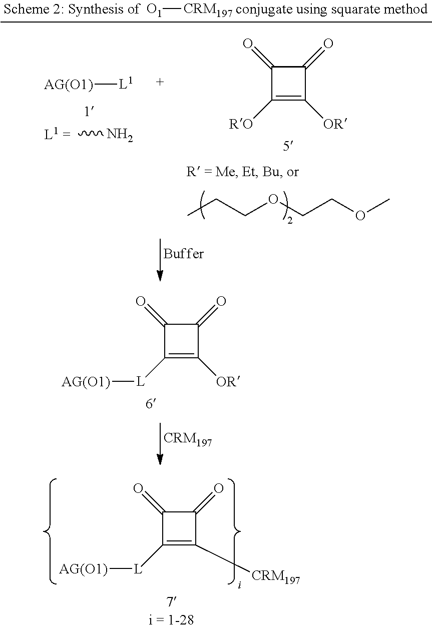

O₁-Antigen 1′ in appropriate solvent (e.g., H₂O-EtOH, buffer) in a vial at rt is treated with desired alkyl squarate 5′ (e.g., 3,4-dibutoxy-3-cyclobutene-1,2-dione, 3,4-(Di(2-(2-(2-methoxyethoxy)ethoxy)ethoxy)-3-cyclobutene-1,2-dione) (Ganesh et al, JACS, 2014, 136, 16260-16269 and Xu et al, Carbhydr. Res, 2018, 456, 24-29) and stirred at rt in solvent with appropriate pH (7-8). The reaction mixture is neutralized using acetic acid and then concentrated (or lyophilized) in vacuum. The crude is purified using C18 (or SEC) column using water-acetonitrile as eluents. The fractions containing product are frozen and lyophilized to afford 6′. The 0.5 M pH 9 borate buffer solution containing Antigen-squarate ester 6′ (25-100 equiv.) and CRM₁₉₇ is stirred at rt for 24-72 h (S. Hou et al, Carbhydr. Res, 2008, 343, 196-210). The resulting O₁-Antigen-CRM₁₉₇ conjugate 7′ is washed, purified, and stored using appropriate buffer solution.

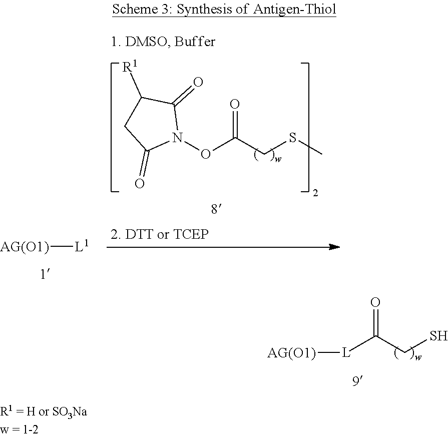

O₁-Antigen 1′ in appropriate solvent (e.g., DMSO) in a vial at rt is treated with 8′ (e.g., DSP (dithiobis(succinimidyipropionate), or DTSSP (3,3′-dithiobis(sulfosuccinimidyipropionate), to obtain the corresponding disulfide, which in turn reduced by DTT (dithiothreitol) or TCEP (tris(2-carboxyethyl)phosphine) to afford the Antigen-thiol 9′.

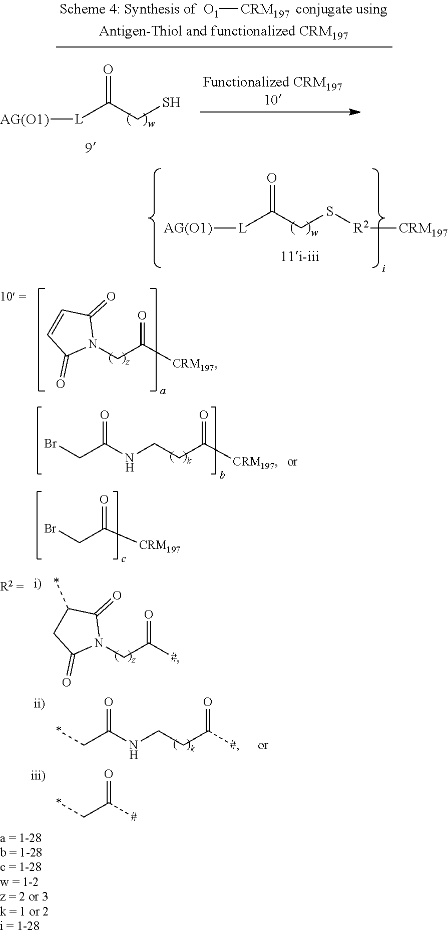

a) Synthesis of O₁-CRM₁₉₇ Conjugate Using Antigen-Thiol-Maleimide Method

The buffer solution containing Antigen-thiol 9′ (25-100 equiv.) and CRM₁₉₇ functionalized with Maleimide 10′ (which can for instance be prepared by treating CRM₁₉₇ with 3-maleimido-propionic acid succinimidyl ester or any other appropriate NHS ester equipped with maleimide, by person skilled in the art) (Robert M. F. van der Put et al, ACS Cent. Sci. 2022, 8, 4, 449-460) is stirred at rt for 20-24 h. Then excess maleimide moieties are quenched by adding L-cysteine in buffer to the RM and stirring for an hour at rt. The resulting O1-Antigen-CRM₁₉₇-thio-maleimide conjugate 11′ is washed, purified, and stored using appropriate buffer solution.

### b) Synthesis of O1-CRM₁₉₇ Conjugate Using Antigen-Thiol-Ether Method

The buffer solution containing Antigen-thiol 9′ (25-100 equiv.) and protein functionalized with α-bromoacetate 10′ (e.g., CRM₁₉₇-BAP, synthesized using CRM₁₉₇ and SBAP (N-succinimidyl 3-(2-bromoacetamido) propanoate) or any other appropriate NHS ester equipped with α-bromoacetate) (Schumann, B. et al, Chem. Sci., 2014, 5, 1992-2002) is stirred at rt for 24 h. Then excess α-bromoacetate moieties are quenched by adding L-cysteine in buffer to the RM and stirring for an hour at rt. The resulting O1-Antigen-CRM₁₉₇-thio-ether conjugate 11′ is washed, purified, and stored using appropriate buffer solution.

### General Retro Synthetic Approach to the O1-Antigen

The O1-antigen can be synthesized as shown in scheme 5 using functionalized building blocks. The completely deprotected antigen RS-1 is equipped with a linker L¹ at its reducing end which is essential for the conjugation with the protein carriers. RS-1 can be accessed from deprotection of the completely protected RS-2. The deprotection strategies may include removal of esters, amide, imide, carbamate via (acidic or basic) hydrolysis, hydrogenolysis, birch reduction, reduction of azide group to amine. The deprotection sequence depends on the protecting groups and their compatibility with reaction conditions. A person skilled in the art is able to accomplish this successfully. RS-2 can be obtained from glycosylation of either RS-3 (Gal II part) as a donor and RS-4 (Gal I part) as an acceptor, or by glycosylating RS-4 (Gal I) with a smaller repeating unit of Gal II part than RS-3, (e.g., disaccharide or tetrasaccharide donor). RS-3 (Gal II part) can be synthesized from the repeating unit RS-5, which in turn can be obtained from glycosylating RS-6 and RS-7.

Removal of LG₃ group of RS-8 yields RS-4 (Gal I part) which is equipped with the appropriate linker handle (Lx). RS-9 donor can be treated with a linker handle of the choice from various linker handles listed in Table 1 below in this reaction to get RS-8. RS-9 can be accessed from the repeating unit RS-10, which is in turn can be obtained from glycosylating RS-11 donor and RS-12 acceptor.

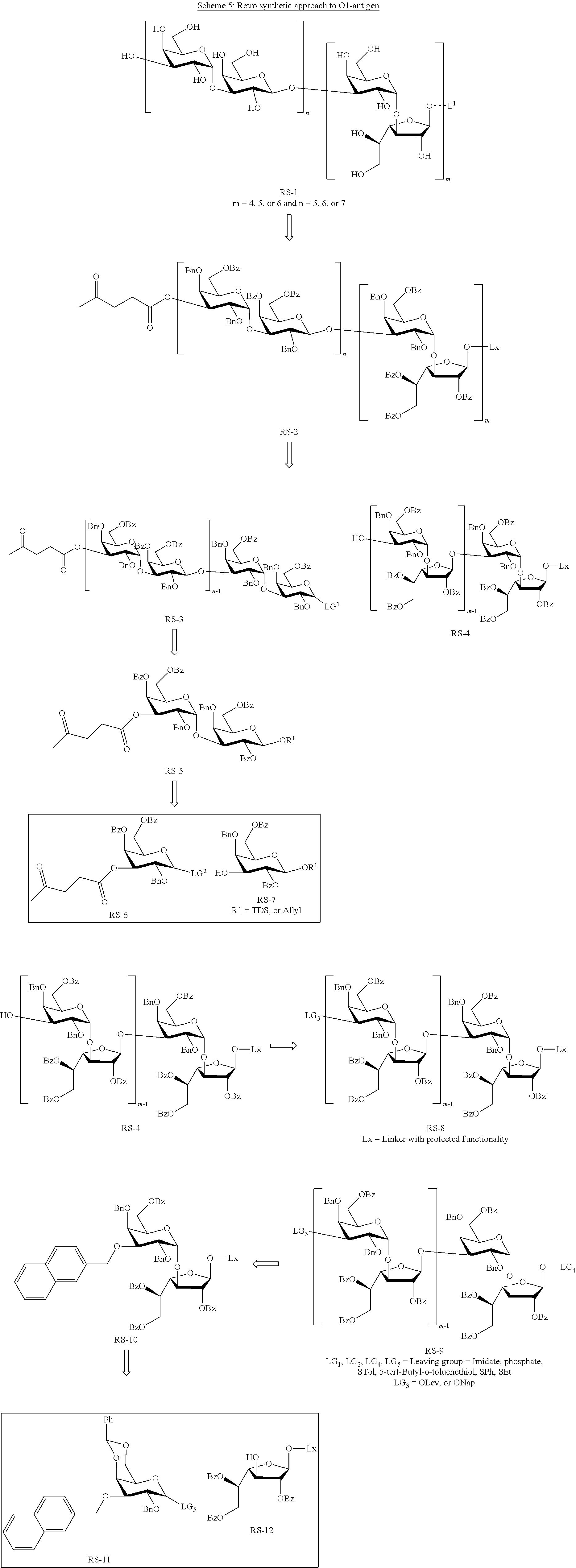

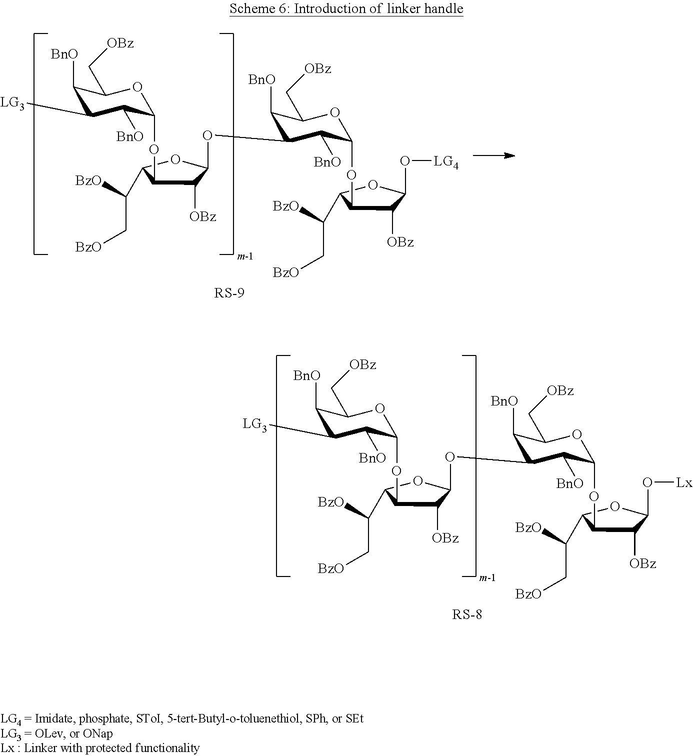

Linker nucleophile Ln (e.g., 5-azidopentan-1-ol) and the RS-9 donor are taken in a RBF and dried azeotropically using dry toluene in the vacuum. The mixture is taken in appropriate solvent (e.g., DCM) at rt, 4 Å molecular sieves is added and it is stirred for 30-45 min under N₂ atmosphere. The RM is cooled to appropriate temperature (e.g., 0° C. to −20° C.) and an activator (e.g., TMSOTf, TfOH) is added to the RM and stirred for 20 mins. The RM is then allowed to warm slowly to room temperature over one h. Reaction completion is monitored by TLC. The RM is quenched (e.g., with sat. NaHCO₃, Na₂S₂O₃ solution), and extracted with solvent (e.g., DCM, EtOAc). Combined organics are washed with water and brine, then dried and evaporated in vacuum to get the crude product. The crude product is purified by silica column chromatography using EA/cyclohexane as eluents. Fractions containing product are evaporated and dried in vacuum to get the product.

TABLE 1
List of nucleophilic linker Ln

HO—(C₂₋₁₀)alkylene-N₃ or HO—(C₂₋₁₀)alkylene-NBnCbz;
HO—(CH₂CH₂O)ₐ—CH₂CH₂—N₃ or HO—(CH₂CH₂O)ₐ—CH₂CH₂—NBnCbz, wherein a is 1, 2 or 3;
HO—CH₂CH₂S—CH₂CH₂N3 or HO—CH₂CH₂S—CH₂CH₂NBnCbz;
HO—(C₂₋₁₀)fluoroalkylene-N₃ or HO—(C₂₋₁₀)fluroalkylene-NBnCbz;
HO—(CH₂)_{c}NHC(O)(CH₂)_{d}—N₃ or HO—(CH₂)_{c}NHC(O)(CH₂)_{d}—NBnCbz, wherein c and d are
independently from each other from 2 to 6;
HO—(CH₂)ₑNHC(O)NH(CH₂)ₕ—N₃ or HO—(CH₂)ₑNHC(O)NH(CH₂)ₕ—NBnCbz, wherein e and
h are independently from each other from 2 to 6;
HO—(C₁₋₁₀)alkylene-C(O)—NH—(C₂₋₁₀)alkylene-N₃ or
HO—(C₁₋₁₀)alkylene-C(O)—NH—(C₂₋₁₀)alkylene-NBnCbz
HO—(C₁₋₁₀)alkylene-C(O)—OR where R is alkyl or benzyl;
HO—(C₂₋₁₀)alkylene-O—NH-^{t}Boc; or HO—(C₂₋₁₀)alkylene-O—N-pthalimide;
HO—(C₂₋₁₀)alkylene-S—Ac

The oligosaccharide part of the claimed compounds, i.e. the range of the claimed lengths thereof, may be prepared as exemplified below in the experimental part, or with analogous methods thereof.

### Experimental Section

Abbreviations (as used herein and in the description above):

| | |
|---|---|
| AcOH | Acetic acid |
| aq. | Aqueous |
| Bn | Benzyl |
| BSA | Bovine serum albumine |
| CDCl₃ | Deuterated chloroform |
| Cs₂CO₃ | Cesium carbonate |
| Cy | Cyclohexane |
| D₂O | Deuterium oxide |
| DCM | Dichloromethane |
| DDQ | 2,3-dichloro-5,6-dicyano-1,4-benzoquinone |
| DMAP | 4-(Dimethylamino)pyridine |
| DMF | N,N-dimethylformamide |
| DMSO | Dimethylsulfoxide |
| ELISA | Enzyme-linked immunosorbent assay |
| equiv | Equivalents |
| ESI | Electrospray ionization |
| Et₃N (TEA) | Triethylamine |
| EtOAc (EA) | Ethyl acetate |
| EtOH | Ethanol |
| EtSH | Ethanethiol |
| Fr | Fraction |
| h | Hours |
| H₂ | Hydrogen |
| H₂O | Water |
| H₂SO₄ | Sulfuric acid |
| HCl | Hydrochloric acid |
| HPLC | High-performance liquid chromatography |
| HPLC-SEC | High-performance liquid chromatography- |
| | size exclusion chromatography |
| I₂ | Iodine |
| ICU | Intensive Care Unit |
| IPA | Isopropanol |
| LPS | Lipopolysaccharide |
| M | Molar |
| MeOH | Methanol |
| Min | Minutes |
| MS | Molecular sieves |
| N₂ | Nitrogen |
| Na | Sodium |
| Na₂S₂O₃ | Sodium thiosulfate |
| Na₂SO₄ | Sodium sulfate |
| NaCl | Sodium chloride |
| NaHCO₃ | Sodium bicarbonate |
| NaOMe | Sodium methoxide |
| NaPi buffer | Sodium phosphate buffer |
| NH₂NH₂ | Hydrazine |
| NIS | N-Iodosuccinimide |
| NMR | Nuclear magnetic resonance spectroscopy |
| PBS | Phosphate-buffered saline |
| PBS-T | Phosphate-buffered saline with 0.1% |
| | (v/v) Tween-20 |
| Pd(OH)₂ | Palladium hydroxide |
| Pd/C | Palladium on carbon |
| py | Pyridine |
| RBF | Round bottom flask |
| RM | Reaction Mixture |
| rt | Room temperature |
| sat. | Saturated |
| SDS-PAGE | Sodium dodecyl sulfate-polyacrylamide |
| | gel electrophoresis |
| SM | Starting material |
| sol. | Solution |
| TBAF | Tetrabutylammonium fluoride |
| TBS | Tris-buffered saline |
| TDS | Dimethyl-Thexylsilylchlorid |
| TLC | Thin layer chromatography |
| TMB | 3,3′,5,5′-Tetramethylbenzidine |
| TMSOTf | Trimethylsilyl trifluoromethanesulfonate |
| UV | Ultraviolet |

### I. Chemistry

The following examples illustrate the preparation of biologically active compounds of the invention but do not at all limit the scope thereof.

### General Information:

All reagents and solvents were used as purchased and solvents used for the reactions were anhydrous. Except reactions containing water as solvent, all reactions were conducted under an atmosphere of N₂ in dried glassware (purchased from VWR and ROTH). Before glycosylation it is highly recommended to dry acceptor and donor by azeotrope with anhydrous toluene twice. Heidolph magnetic stirrer was used to carry out the experiments. Thin-layer chromatography (TLC) was performed on silica gel 60 F254 glass plates (Merck) or aluminium plates (VWR). Developed TLC plates were visualized under a short-wave UV lamp and by heating plates that were dipped in sugar stain solution (3-methoxy phenol (0.225 mL), H₂SO₄ (6 mL) and EtOH (200 mL)). All automated flash chromatography purifications on silica gel (FlashPure Silica 40 μm irregular: BUCHI columns) were carried out with Biotage Isolera and Biotage Select. BUCHI rotary evaporator was used to evaporate the solvent. Dry ice and acetone and ice/water combination were used for cooling the reaction mixture to get the desired temperatures. All NMR experiments were carried out on BRUKER 400 MHz instrument.

Temperatures are indicated in degrees Celsius (° C.). In mixtures, relations of parts of solvent or eluent or reagent mixtures in liquid form are given as volume relations (v/v), unless indicated otherwise.

### Characterisation Methods Used:

HPLC-SEC: The glycoconjugates used for immunizations were analyzed by HPLC-SEC to observe mass differences between conjugated and unconjugated CRM₁₉₇ proteins. The samples were diluted in 50 mM Tris, 20 mM NaCl, pH 7.2 and run on an Agilent 1100 HPLC system fitted with Tosoh TSK G2000 column (SWxl, 7.8 mm×30 cm, 5 μm) and a Tosoh TSK gel Guard column (SWxl 6.0 mm×4 cm, 7 μm). The flow rate was kept at 1 mL/min.

SDS-PAGE: The samples were diluted in Laemmli loading buffer and heated for 5 min at 95° C. After cooling at RT for 5 min, approximately 2-2.5 μg of the samples were loaded into the wells of a 10% polyacrylamide gel along with approx. 5 μL of the protein size marker. The samples were run at a constant voltage of 120 V for approximately 30-45 min. Staining was done using the Gel Code™ Blue Safe Protein Stain as per manufacturer's instructions. The gels were washed with deionized water overnight and scanned.

### Synthesis of thexyldimethylsilyl 4,6-O-benzylidene-2-O-benzyl-3-O-naphtylmethyl-α-D-galactopyranosyl-(1→3)-2,5,6-tri-O-benzoyl-β-D-galactofuranoside A3

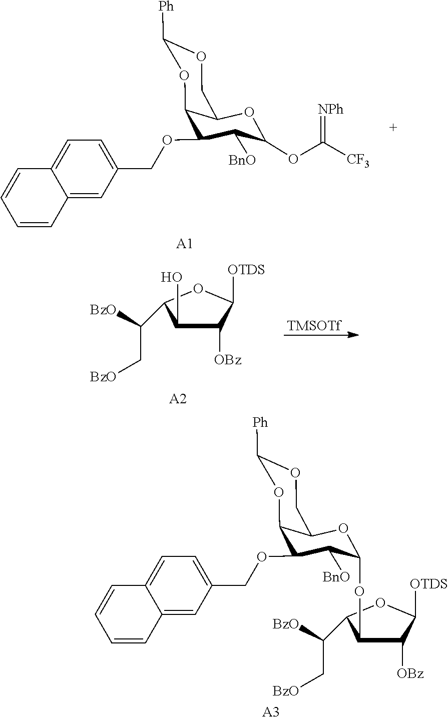

A2 (see WO2019106201, page 164) (51.0 g, 80 mmol) and A1 (see WO2019106201, page 162) (61.9 g, 92 mmol) were dissolved in anhydrous Toluene (3×100 mL), dried azeotropically and the residue dried at high vacuum for an hour. The dried mixture dissolved in anhydrous toluene (750 ml) and Dioxane (250 mL) at rt, added 4 Å molecular sieves and stirred at for 45 min under N₂ atmosphere. Cooled the RM to 0° C. using Ice water bath and added TMSOTf (1.45 mL, 8.03 mmol) to the RM and stirred the RM at 5° C. for 20 min. RM was then allowed to warm slowly to room temp over one hr. TLC analysis (20% EA/Cyclohexane) showed the completion of the reaction. RM was quenched with sat. NaHCO₃ (250 mL), stirred for 10 min. Extracted with EA (200 mL×3). Combined organics were washed with water (100 mL), brine (50 mL), dried (Na₂SO₄), evaporated in vacuum to get the crude product. Column purification on silica was done using EA/cyclohexane on biotage and the product was evaporated and dried in high vacuum to obtain white gummy solid A3 (68.4 g, 76%). HRMS (ESI+) Calcd for C₆₆H₇₀O₁₄NaSi⁺ [M+Na]⁺ 1137.4427, found 1337.4432.

### Synthesis of thexyldimethylsilyl 2,4-di-O-benzyl-3-O-naphtylmethyl-α-D-galactopyranosyl-(1→3)-2,5,6-tri-O-benzoyl-8-D-galactofuranoside A4

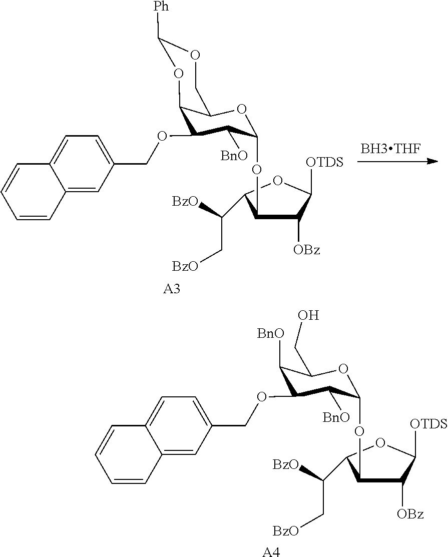

A3 (47 g, 42.1 mmol) was taken in THF (250 mL), added dried 4 Å molecular sieves to it and stirred at rt for 15 min. Added 1M BH₃-THF solution (169 mL, 169 mmol) to the RM and stirred for 5 min before the addition of the TMSOTf (0.76 mL, 4.21 mmol) and stirred at rt for 16 h. TLC analysis showed the completion of the reaction. RM was quenched with methanol (35 mL) slowly (careful, effervescence) at rt and stirred for 45 mins. and then diluted with sat. NaHCO₃ solution (250 mL) and EA (300 mL). Stirred the RM well for 2 hrs. Diluted with water (200 mL). Separated the layers. The aqueous layer was extracted with EA (100 mL×3). Combined organics were washed with brine solution (100 mL), dried (Na₂SO₄), filtered, and evaporated in vacuum to get colorless gummy liquid. Crude product was column purified using EtOAc/Cyclohexane and product eluted with 20-30% EtOAc/Cy, evaporation of fractions containing product spots in rotary evaporator yielded the colorless gummy liquid A4 (29.8 g, 63%). HRMS (ESI+) Calcd for C₆₆H₇₂O₁₄NaSi⁺ [M+Na]⁺ 1139.4584, found 1339.4583.

### Synthesis of thexyldimethylsilyl 6-O-benzoyl-2,4-di-O-benzyl-3-O-naphtylmethyl-α-D-galactopyranosyl-(1→3)-2,5,6-tri-O-benzoyl-β-D-galactofuranoside A5

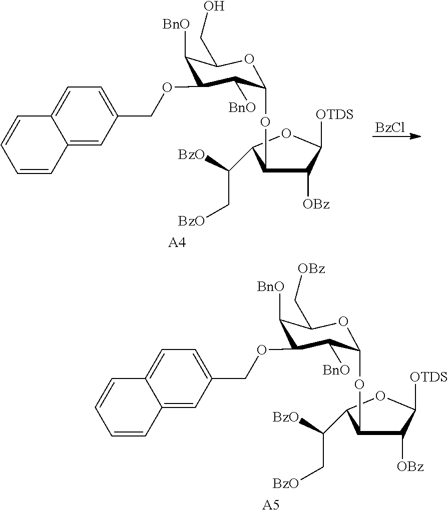

A4 (29.8 g, 26.7 mmol) was taken in DCM (300 mL) at rt, added pyridine (10.79 mL, 133 mmol) and DMAP (0.33 g, 2.67 mmol) to it and stirred for 5 min. Then added BzCl (6.19 mL, 53.3 mmol) to it and stirred for 18 h. TLC analysis (20% EA/Cy) showed completion of the reaction. RM was diluted with NaHCO₃ (100 mL), separated the layers. The aqeuous layer was extracted with DCM (100 mL×2). The combined organic layer was washed with brine solution (50 mL), dried (Na₂SO₄), filtered and evaporated in vacuum to get pale brown residue. Purified by biotage using silica column and EtOAc and cyclohexane as eluents, fractions containing product spots were collected and evaporated in vacuum and dried in high vacuum to get white gummy liquid A5 (28 g, 86%). HRMS (ESI+) Calcd for C₇₃H₇₆O₁₅NaSi⁺ [M+Na]⁺ 1243.4846, found 1243.4827.

### Synthesis of thexyldimethylsilyl 6-O-benzoyl-2,4-di-O-benzyl-α-D-galactopyranosyl-(1→3)-2,5,6-tri-O-benzoyl-β-D-galactofuranoside A6

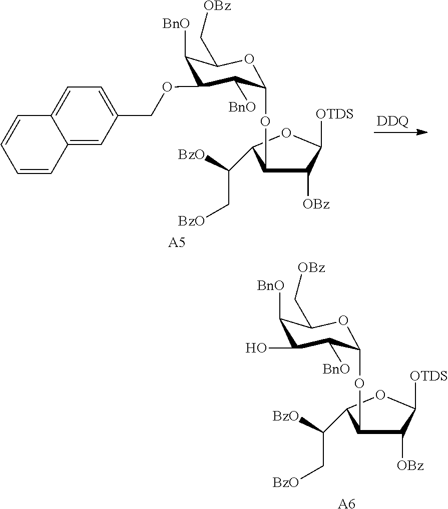

Substrate A5 (14 g, 11.46 mmol) was taken in DCM (150 mL) and PBS buffer solution (300 mL) at rt, added DDQ (5.2 g, 22.92 mmol) in portions over 1 h, RM became black then it turned to reddish brown colour and stirred for 3 h. TLC analysis (20% EA/Cy) showed the presence of polar spot and little SM. So, continued stirring for 2 h more. RM was quenched with NaHCO₃ solution (250 mL) and extracted with DCM (100 mL×3). Combined organics were washed with NaHCO₃ solution (250 mL), brine solution (100 mL), dried (Na₂SO₄), filtered, concentrated in vacuum to get crude. The crude solid was triturated with methanol and filtered. The residue was washed with methanol to get white solid which was dried in high vacuum (fr1, 7.5 g) and the mother liquor was evaporated in vacuum to get yellow solid and trituration with methanol yielded second crop as white solid (2.37 g). So, the total yield of A6 was 9.85 g (80%). HRMS (ESI+) Calcd for C₆₂H₆₈O₁₅NaSi⁺ [M+Na]⁺ 1103.4220, found 1103.4225.

### Synthesis of thexyldimethylsilyl 6-O-benzoyl-2,4-di-O-benzyl-3-O-levulinoyl-α-D-galactopyranosyl-(1→3)-2,5,6-tri-O-benzoyl-8-D-galactofuranoside A7

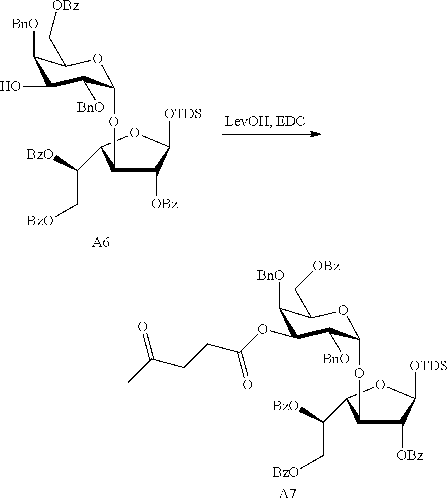

Substrate A6 (21 g, 19.17 mmol) was taken in DCM (200 mL) at rt, added LevOH (6.68 g, 57.5 mmol), DMAP (0.47 g, 3.83 mmol), DIPEA (50.2 mL, 288 mmol) to it and stirred for 5 min. Then added HOBt (2.94 g, 19.17 mmol) and EDC·HCl (18.38 g, 96 mmol) to it and stirred at rt overnight. TLC analysis (20% EA/Cy) showed the presence of polar spot. So, RM was diluted with DCM (500 mL) and washed with dil aq. HCl (250 mL×2), then with sat. NaHCO₃ solution (250 mL×2). Organic layer was washed with brine solution (200 mL), dried (Na₂SO₄), filtered, concentrated in vacuum to get crude product as brown oil. Purified by silica column chromatography using EA and cyclohexane as eluents, fractions containing pure product spots were collected separately and evaporated in vacuum and dried in high vacuum to get off-white fluffy solid A7 (20.3 g, 89%). HRMS (ESI+) Calcd for C₈₇H₇₄O₁₇NaSi⁺ [M+Na]⁺ 1201.4587, found 1201.4596.

### Synthesis of 6-O-benzoyl-2,4-di-O-benzyl-3-O-levulinoyl-α-D-galactopyranosyl-(1→3)-2,5,6-tri-O-benzoyl-β-D-galactofuranoside A8

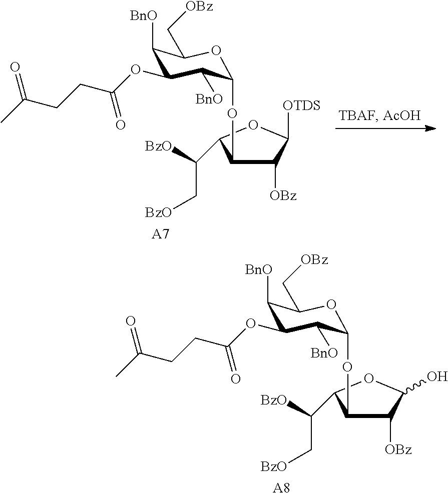

Substrate A7 (29.2 g, 24.76 mmol) was taken in DCM (250 mL) in 1 L RBF, at rt, added AcOH (22 mL, 384 mmol) to it and stirred for 5 mins. Then added 1M TBAF in THF (371 mL, 371 mmol) to the RM and stirred at rt for 24 h. TLC analysis showed the completion of the reaction. So, diluted with water (200 mL) and DCM (100 mL). Separated the layers. The aqueous layer was extracted with DCM (200 mL×2). The combined organic layer was washed with water (250 mL), sat. NaHCO₃ solution (250 mL), brine solution (250 mL), dried (Na₂SO₄), filtered, evaporated in vacuum. Purified by biotage using silica column and EA and cyclohexane as eluents, fractions containing product spots were collected and evaporated in vacuum and dried in high vacuum to get off-white gummy solid A8 (22 g, 86%). HRMS (ESI+) Calcd for C₅₉H₅₆O₁₇Na⁺ [M+Na]⁺ 1059.3410, found 1059.3411.

### Synthesis of 6-O-benzoyl-2,4-di-O-benzyl-3-O-levulinoyl-α-D-galactopyranosyl-(1→3)-2,5,6-tri-O-benzoyl-β-D-galactofuranosyl 2,2,2-trifluoro-N-phenylacetimidate A9

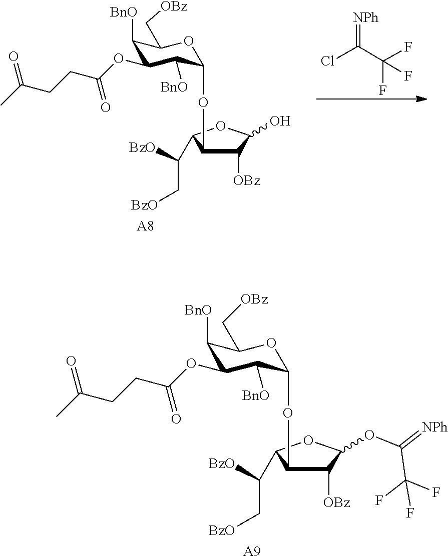

Hemiacetal A8 (21.1 g, 20.4 mmol) was taken in DCM (100 mL) at rt under N₂ atmosphere, added Cs₂CO₃ (26.5 g, 81 mmol) to it and stirred for 5 mins. Then added (E)-2,2,2-trifluoro-N-phenylacetimidoyl chloride (12.7 g, 9.7 mL, 61 mmol) to it and stirred overnight. TLC analysis showed that the reaction was complete, and no SM was present. So, RM was filtered through celite to remove the solid, washed the residue with DCM (100 mL×4). The Filtrate was concentrated in vacuum and co-evaporated with toluene (100 mL) thrice. On evaporation and drying under vacuum pale yellowish colored fluffy solid was obtained A9 (23.11 g, 94%). HRMS (ESI+) Calcd for C₈₇H₈₄F₃N₂O₁₇⁺ [M+NH₄]⁺ 1225.4152, found 1225.4132.

### Synthesis of thexyldimethylsilyl 6-O-benzoyl-2,4-di-O-benzyl-3-O-levulinoyl-α-D-galactopyranosyl-(1→3)-2,5,6-tri-O-benzoyl-β-D-galactofuranosyl-(1→3)-6-O-benzoyl-2,4-di-O-benzyl-3-O-levulinoyl-α-D-galactopyranosyl-(1→3)-2,5,6-tri-O-benzoyl-β-D-galactofuranoside A10

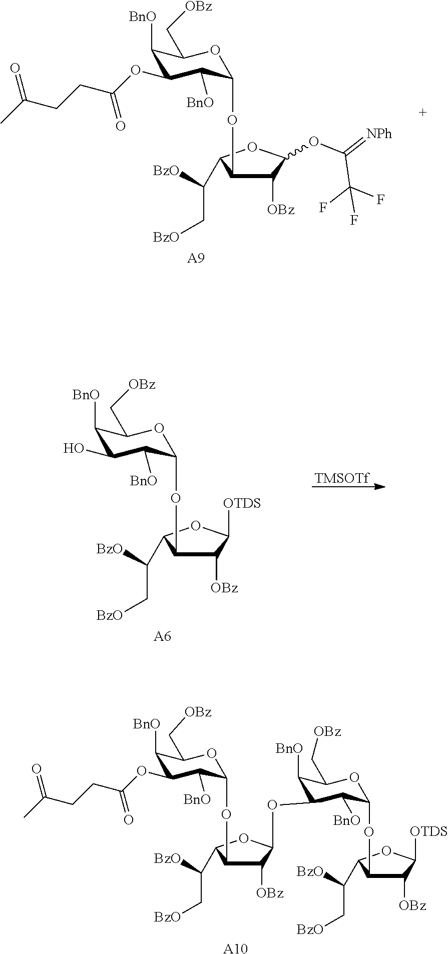

Acceptor A6 (17.5 g, 16.18 mmol) and Imidate donor A9 (23.46 g, 19.42 mmol) were taken in DCM (270 mL) at rt, added 4 A molecular sieves to it and stirred for stirred at rt for 45 min under N₂ atmosphere. Cooled the RM to −10° C. using ice-acetone bath and added TMSOTf (1.23 mL, 3.24 mmol) to the RM and stirred the RM at 0° C. for 15 mins slowly warmed to 5° C. over one h. TLC analysis (25% EA/Cy) showed that the reaction was complete, absence of the acceptor SM and presence of a slightly polar spot. RM was quenched with sat. NaHCO₃ solution (100 mL), separated the layers, extracted the aqueous layer with DCM (100 mL×2). Combined organic layer was washed with sat. NaHCO₃ solution (100 mL), brine solution (100 mL), dried (Na₂SO₄), filtered, and evaporated in vacuum. Purified by silica gel column chromatography using EA/Cy to get pure product as white fluffy solid A10 (28 g, 82%). MALDI-TOF Calcd for C₁₂₁H₁₂₂NaO₃₁Si⁺ [M+Na]⁺ 2121.7632, found 2121.883.

### Synthesis of Tetrasaccharide Acceptor A11

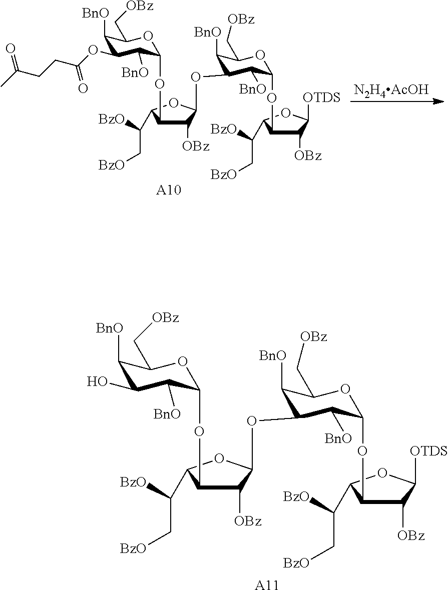

Lev-substrate A10 (28 g, 13.33 mmol) was taken in DCM (162 mL)-Pyridine (16 mL) at rt, added hydrazine acetate (6.14 g, 66.7 mmol) to it and stirred at rt for 18 h. TLC showed the presence of a sugar active spot slightly non-polar to the Rf value of the SM in 30% EA/Hexanes. RM was then quenched with acetone (10 mL) and stirred for 45 mins at rt. The RM was then evaporated to dryness in vacuum. The residue was purified using silica column chromatography with EA-Cy as eluents to get the sugar active spot, on evaporation and drying in the high vacuum the desired compound was obtained as white fluffy solid A11 (24.4 g, 91%). MALDI-TOF Calcd for C₁₁₆H₁₁₈NaO₂₉S⁺ [M+Na]⁺ 2023.7269, found 2024.259.

### Synthesis of Tetrasaccharide Hemiacetal A12

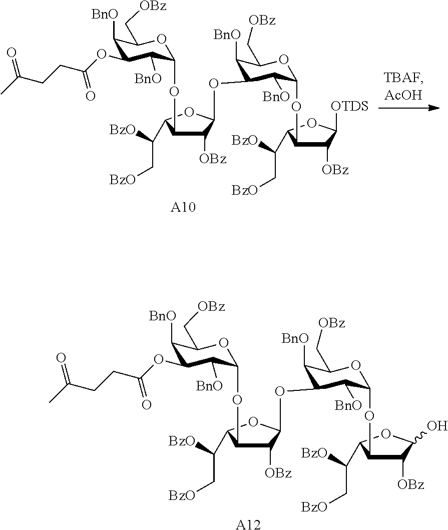

Substrate A10 (28 g, 13.33 mmol) was taken in DCM (135 mL) in a 1 L RBF, at rt, added AcOH (12.3 mL, 213 mmol) to it and stirred for 5 mins. Then added 1M TBAF in THF solution (200 mL, 200 mmol) to RM and stirred at rt for 20 h. TLC analysis showed almost completion of the reaction. So, diluted with water (200 mL) and DCM (100 mL). Separated the layers. The aqueous layer was extracted with DCM (50 mL×2). The combined organic layer was washed with water (100 mL), sat. NaHCO₃ solution (100 ml×2), brine solution (100 mL), dried (Na₂SO₄), filtered, evaporated in vacuum. Purified using silica column and EA and Cy as eluents, fractions containing product spot were evaporated in vacuum and dried in high vacuum to get white fluffy solid A12 (23 g, 88%). MALDI-TOF Calcd for C₁₁₃H₁₀₄NaO₃₁⁺ [M+Na]⁺ 1979.6454, found 1979.764.

### Synthesis of Tetrasaccharide Imidate Donor A13

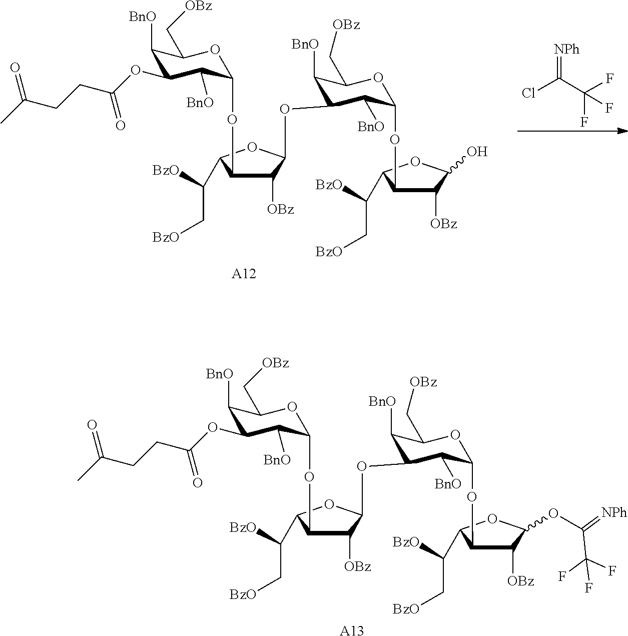

Hemiacetal A12 (25.7 g, 13.13 mmol) was taken in DCM (130 mL) at rt under N₂ atmosphere, added Cs₂CO₃ (17.11 g, 52.5 mmol) to it and stirred for 5 mins. Then added (E)-2,2,2-trifluoro-N-phenylacetimidoyl chloride (8.17 g, 6.2 mL, 39.4 mmol) to it and stirred overnight. TLC analysis showed that the reaction was complete and intense nonpolar spot was present and no SM was present. So, RM was filtered through celite to remove the solid, washed the residue with DCM (100 mL×4). The Filtrate was concentrated in vacuum and co-evaporated with toluene (100 mL) thrice. On evaporation and drying under vacuum pale yellowish colored fluffy solid was obtained A13 (27.9 g, quantitative).

### Synthesis of Octasaccharide A14

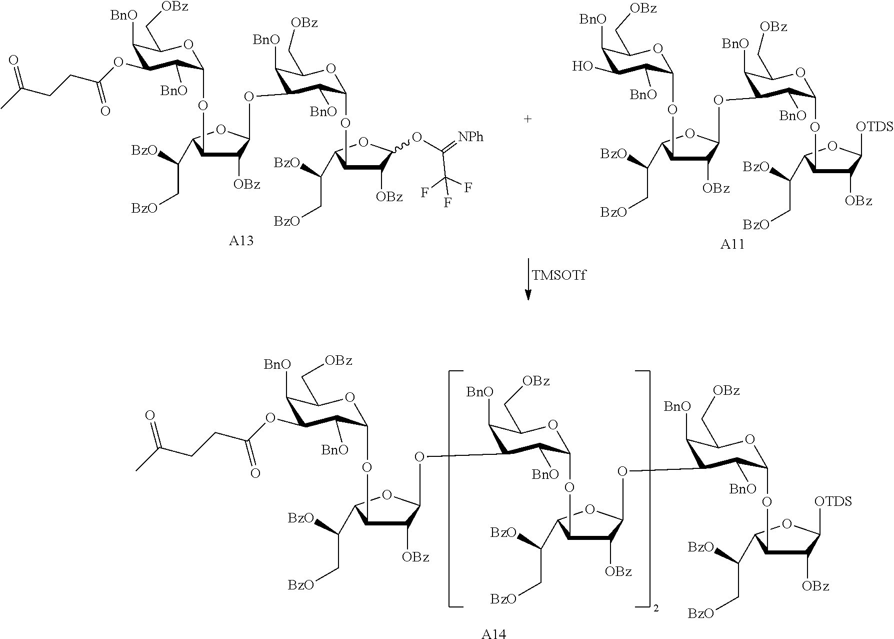

Both the acceptor A11 (11.9 g, 5.78 mmol) and the donor A13 (13.9 g, 6.36 mmol) were taken in RBF and dried azeotropically using dry toluene in the vacuum. Mixture was taken in DCM (130 mL) at rt, added 4 A molecular sieves to it and stirred for 45 min under N₂ atmosphere. Cooled the RM to −10° C. and added TMSOTf (0.2 mL, 1.16 mmol) to the RM and stirred the RM at −5° C. for 20 mins. RM was then allowed to warm slowly to room temp over one hr. TLC analysis (30% EA/Cy) showed that a slightly polar product intense spot was present. RM was quenched with sat. NaHCO₃ (250 mL), stirred for 10 mins and extracted with DCM (100 mL×3). Combined organics were washed with water (100 mL), brine (100 mL), dried (Na₂SO₄), evaporated in vacuum to get crude product. Column purification was done using EA/Cy on biotage using silica column. Fractions containing product were evaporated and dried in vacuum to get desired product as a white foamy solid A14 (21.8 g, 93%). MALDI-TOF Calcd for C₂₂₉H₂₁₈NaO₅₉Si⁺ [M+Na]⁺ 3962.3720, found 3963.910.

### Synthesis of Octasaccharide Hemiacetal A15

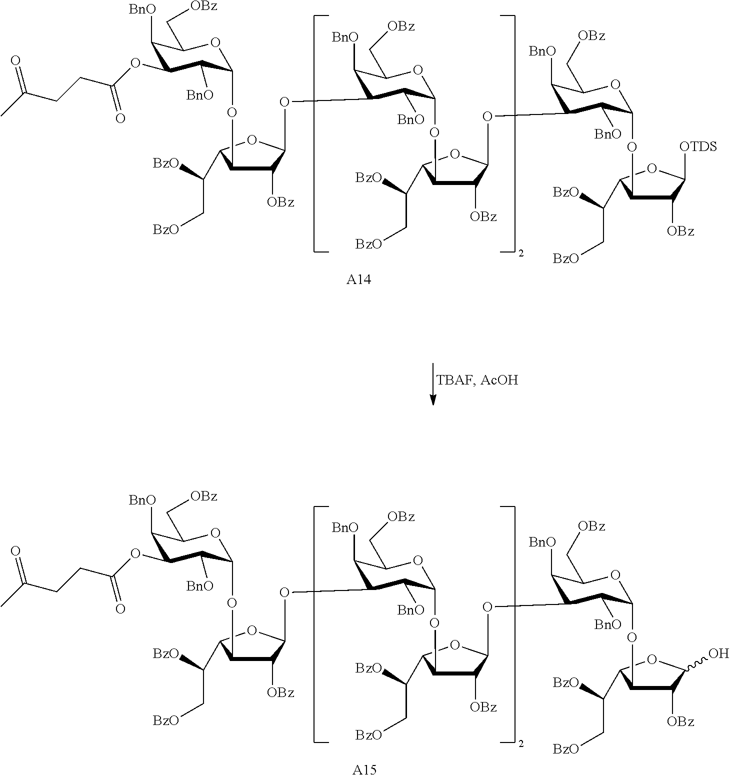

Substrate A14 (21.6 g, 5.48 mmol) was taken in DCM (55 mL) in 500 mL RBF, at rt, added AcOH (4.9 mL, 85 mmol) to it and stirred for 5 mins. Then added TBAF (82 mL, 82 mmol) to it. RM was stirred at rt for 24 h. TLC analysis showed the completion of the reaction. So, diluted with water (200 mL) and DCM (100 mL). Separated the layers. The aqueous layer was extracted with DCM (100 mL×2). The combined organic layer was washed with sat. NaHCO₃ solution (100 ml×2), brine solution (100 ml), dried (Na₂SO₄), filtered, evaporated in vacuum. Purified using silica column on Biotage using EA and Cy as eluents, fractions containing sugar stain active product spots were collected, evaporated in vacuum, and dried in high vacuum to get off-white solid as desired product A15 (17.8 g, 85%). MALDI-TOF Calcd for C₂₂₁H₂₀₀NaO₅₉⁺ [M+Na]⁺ 3820.2542, found 3821.537.

### Synthesis of Octasaccharide Imidate Donor A16

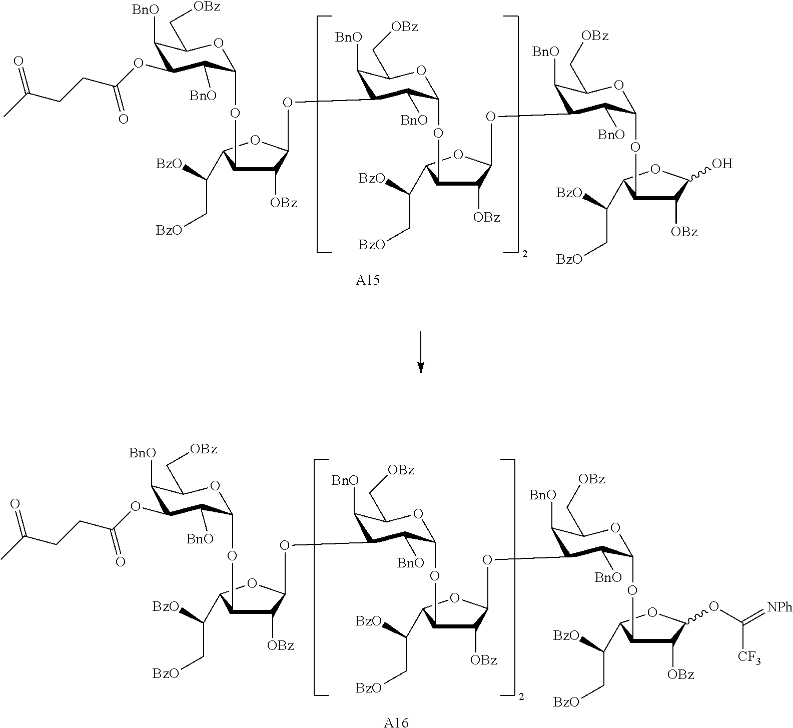

Hemiacetal A15 (17.6 g, 4.6 mmol) was taken in DCM (100 mL) at rt under N₂ atmosphere, added CS₂CO₃ (6.0 g, 18.5 mmol) to it and stirred for 5 mins. Then added (E)-2,2,2-trifluoro-N-phenylacetimidoyl chloride (2.9 g, 2.2 mL, 13.9 mmol) to it and stirred overnight. TLC analysis showed that the reaction was complete and intense nonpolar spot was present and no SM was present. So, RM was filtered through celite to remove the solid, washed the residue with DCM (100 mL×4). The Filtrate was concentrated in vacuum and co-evaporated with toluene (100 mL) thrice. On evaporation and drying under vacuum pale yellowish colored fluffy solid was obtained A16 (18.3 g, quantitative).

### Synthesis of Octasaccharide with Linker A17

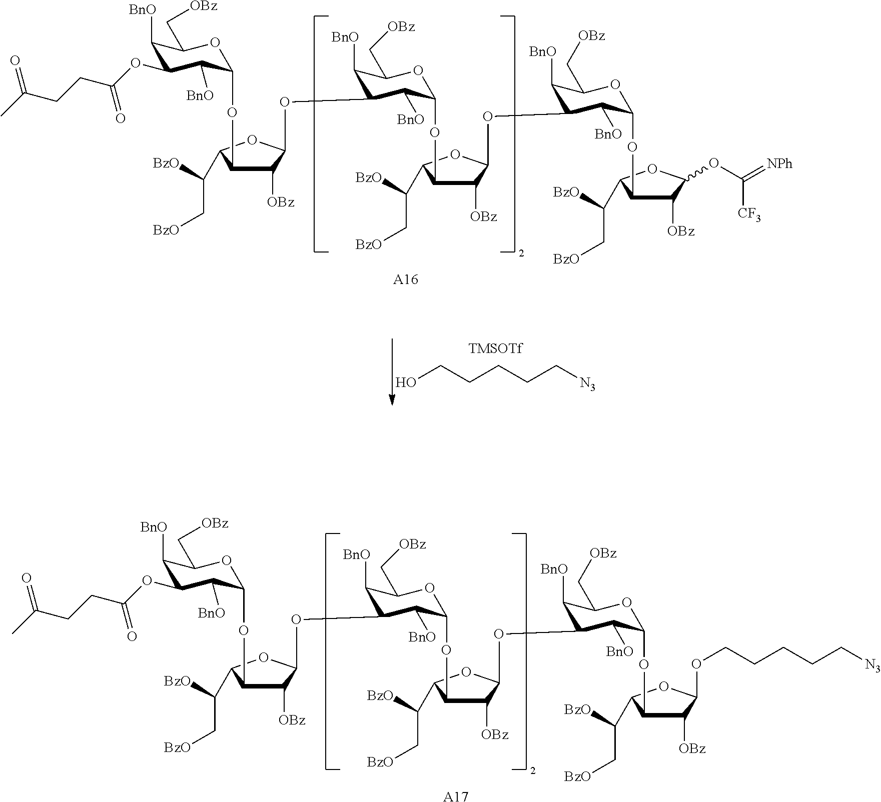

5-azidopentan-1-ol (1.661 g, 12.86 mmol) and the imidate donor A16 (17.02 g, 4.29 mmol) were taken in RBF and dried azeotropically using dry toluene in the vacuum. Mixture was taken in DCM (425 mL) at rt, added 4 A molecular sieves to it and stirred for 30 min under N₂ atmosphere. Cooled the RM to −7° C. and added TMSOTf (0.155 ml, 0.857 mmol) to the RM and stirred the RM at −5° C. for 20 mins. RM was then allowed to warm slowly to room temp over one hr. RM was quenched with sat. NaHCO₃ (250 mL), stirred for 10 mins and extracted with DCM (100 mL×3). Combined organics were washed with water (100 mL), brine (100 mL), dried (Na₂SO₄), evaporated in vacuum to get crude. Column purification was done using EA/cyclohexane on biotage using silica column. Fractions containing product were evaporated and dried in vacuum to get product as a white foamy solid A17 (13.14 g, 78%). MALDI-TOF Calcd for C₂₂₆H₂₁₀N₃O₅₉⁺ [M+H]⁺ 3909.3519, found 3910.123.

### Synthesis of Octasaccharide Acceptor A18

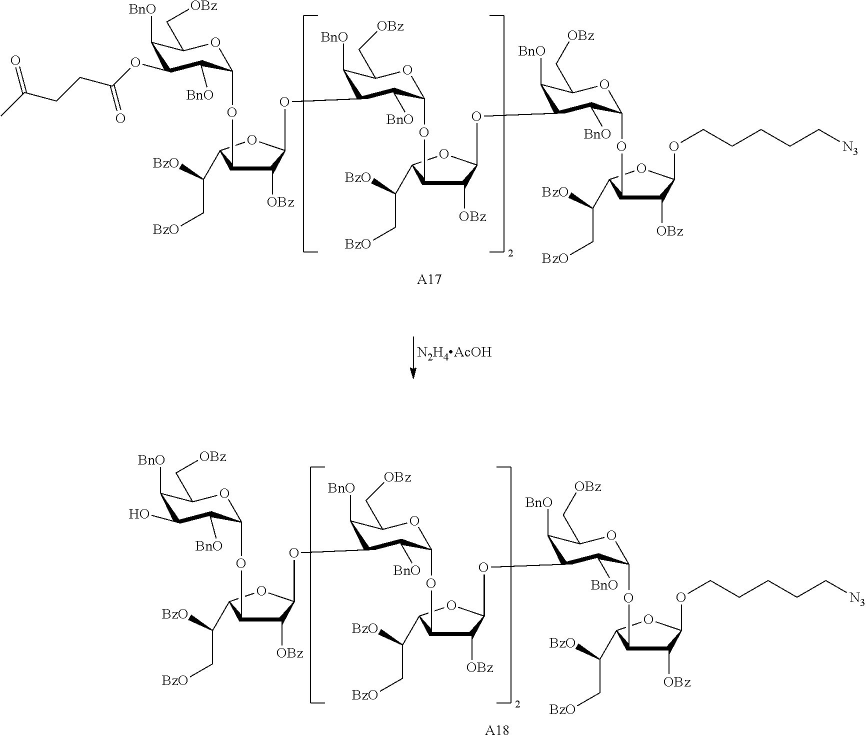

To a solution of the starting material A17 (7.8 g, 1.99 mmol) in DCM (50 mL), a solution of hydrazine hydrate ((0.26 g, 7.98 mmol), dissolved in acetic acid (4.34 mL, 76 mmol) and pyridine (6.45 mL, 80 mmol), was added. The resulting reaction mixture was stirred at rt for 18 h. The reaction was quenched by the addition of acetone (3 mL), stirred for 1 h and the solvent removed under vacuum to obtain the crude product. The crude product was purified on silica column using automated flash chromatography with EA-Cyclohexane as eluents to get the sugar active spot which on evaporation and drying in the high vacuum yielded the desired compound as white fluffy solid A18 (6.97 g, 92%). MALDI-TOF Calcd for C₂₂₁H₂₀₄N₃O₅₇⁺ [M+H]⁺ 3811.3151, found 3811.774.

### Synthesis of B2

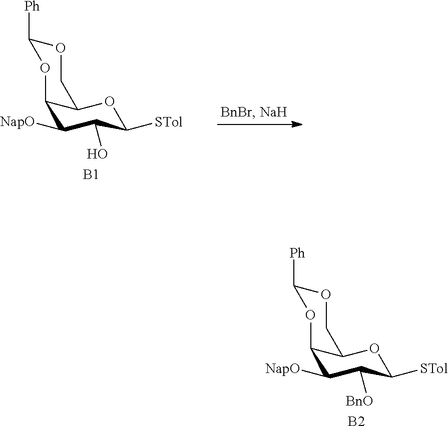

To a stirred solution of compound B1 (Angew. Chem. Int. Ed., 2011, 50. 7315) (72.4 g, 0.1406 mol) in DMF (724 mL), NaH (5.0 g, 0.2250 mol) was added portion wise at 0° C., followed by BnBr (38.50 g, 0.2110 mol) drop wise and the reaction mixture was allowed to stir rt for 14 h. After completion of the reaction was confirmed by TLC, the reaction mixture was poured into ice water, the precipitated solid was filtered, washed with 5% ethyl acetate in hexane and dried under vacuum to afford compound B2 (79.1 g, 93%) as colourless solid.

### Synthesis of B3

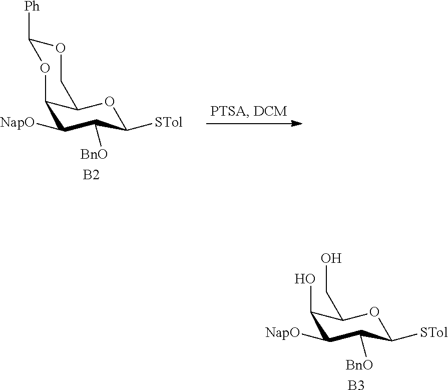

To a stirred solution of compound B2 (79.1 g, 0.1308 mol) in DCM:MeOH (1:1, 1.582 L) at RT, was added P-TSA (24.98 g, 0.1308 mol) and the reaction mixture was allowed to stir at rt for 12 h. After the completion of the reaction was confirmed by TLC and HPLC, the reaction mixture was quenched with ice cold water. The volatiles were removed under vacuum and the residue was diluted with water and was extracted with DCM (2×500 mL). The combined organic layer was dried over Na₂SO₄ and evaporated under reduced pressure. The crude compound thus obtained was purified by column chromatography over silica gel eluting with 35 to 40% of ethyl acetate in hexane to afford compound B3 as gummy liquid (50.6 g, 75%).

### Synthesis of B4

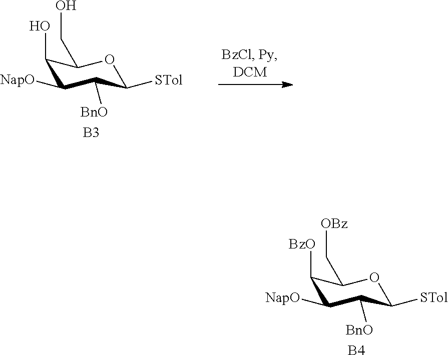

To the stirred solution of compound B3 (50.6 g, 0.098 mol) in DCM (506 mL) at rt were added pyridine (27.11 g, 0.3432 mol) followed by BzCl (48.24 g, 0.3432 mol) drop wise and DMAP (2.39 g, 0.0196 mol) at 0° C. The resultant reaction mixture was allowed to stir at rt for 14 h. After the completion of the reaction was monitored by TLC, the reaction mixture was quenched with ice cold water, extracted with DCM (2×400 mL). The combined organic layer was washed with 5% citric acid solution, water and dried over Na₂SO₄ and was evaporated under reduced pressure. The crude compound thus obtained was washed with 20% EtOAC in Hexane to afford compound B4 (57.5 g, 81%) as off-white solid.

### Synthesis of B5

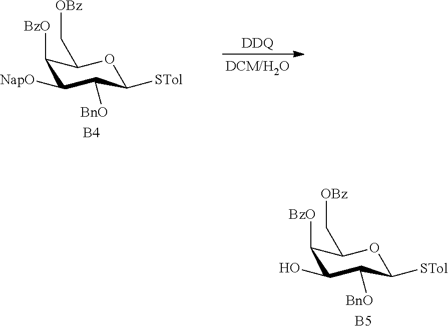

To a stirred solution of Compound B4 (57.5 g, 0.0794 mol) in DCM:Water (9:1, 1.15 L) was added DDQ (36 g, 0.1588 mol) portion wise at 0° C. Then reaction mixture was allowed to stir at rt for 6 h. After the completion of the reaction was confirmed by TLC and LCMS, the reaction mixture was filtered over celite bed. The filtrate was diluted with DCM and washed with sodium thiosulphate and saturated NaHCO₃ solution. The organic layer was dried over Na₂SO₄ and evaporated under reduced pressure. The crude thus obtained was purified by column chromatography over silica gel eluting with 15-20% of ethyl acetate in hexane to afford compound B5 (40.3 g, 87%) as gummy liquid.

### Synthesis of B6

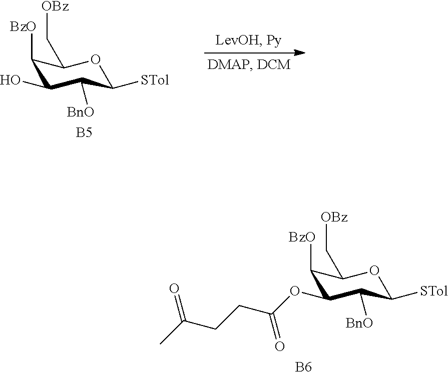

To a stirred solution of compound B5 (40.3 g, 0690 mol) in DCM (806 mL) at 0° C., was added LevOH (9.6 g, 0.0828 mol) was drop wise added, followed by DIPC (11.31 g, 0.0897 mol) and DMAP (2.53 g, 0.0207 mol) at same temperature. The reaction mixture was allowed to stir at rt for 5 h. After the completion of the reaction was confirmed by TLC, the reaction mixture was quenched with ice cold water and extracted with DCM. The organic layer was dried over Na₂SO₄ and evaporated under reduced pressure. The crude compound thus obtained was purified by column chromatography over silica gel eluting with 10-15% of ethyl acetate in hexane to afford compound B6 (36.27 g, 77%) as colourless gum. 1H NMR (400 MHz, Chloroform-d) δ 7.99 (ddd, J=8.5, 3.1, 1.4 Hz, 4H), 7.74-7.24 (m, 14H), 7.07 (d, J=8.0 Hz, 2H), 5.74 (dd, J=3.4, 1.1 Hz, 1H), 5.17 (dd, J=9.6, 3.3 Hz, 1H), 4.84 (d, J=10.8 Hz, 1H), 4.74 (d, J=9.7 Hz, 1H), 4.63 (d, J=10.8 Hz, 1H), 4.57 (dd, J=11.4, 7.0 Hz, 1H), 4.34 (dd, J=11.4, 5.8 Hz, 1H), 4.14 (ddd, J=6.9, 5.6, 1.1 Hz, 1H), 3.80 (t, J=9.6 Hz, 1H), 2.88-2.36 (m, 4H), 2.34 (s, 3H), 2.07 (s, 3H).

### Synthesis of B7

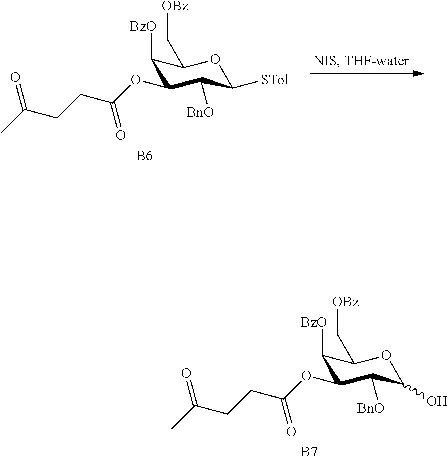

To a stirred solution of compound B6 (36.27 g, 0.0531 mol) in DCM; Water (9:1, 725.4 mL) at 0° C., was added NIS (35.85 g, 0.1593 mol) portion wise and was allowed to stir at rt for 3 h. After the completion of the reaction was confirmed by TLC, the reaction mixture was evaporated under reduced pressure to remove the volatiles and the crude thus obtained was diluted DCM, washed with sodium thiosulphate and brine solution, the organic layer was dried over Na₂SO₄ and evaporated under vacuum. The crude compound was purified by column chromatography over silica gel eluting with 20-25% of ethyl acetate in hexane to afford compound B7 (25.4 g, 83%) as pale-yellow gummy liquid.

### Synthesis of B8

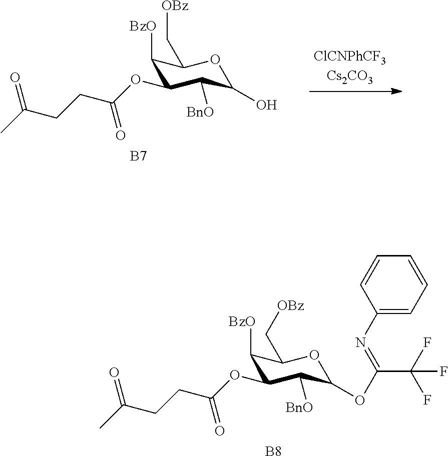

To a stirred solution of compound B7 (25.4 g, 0.0440 mol) in DCM (508 mL) at 0° C. was added DSK-457E (22.88 g, 0.1101 mol) followed by Cesium Carbonate (42.8 g, 0.1321 mol) and was allowed to stir at rt for 8 h. After the completion of the reaction was confirmed by TLC, the rm was filtered through a celite bed, the filtrate was evaporated under reduced pressure and the crude thus obtained was washed with hexane at −20° C. and dried to afford compound B8 as pale-yellow gummy liquid (23.38 g, 71%).

### Synthesis of B9

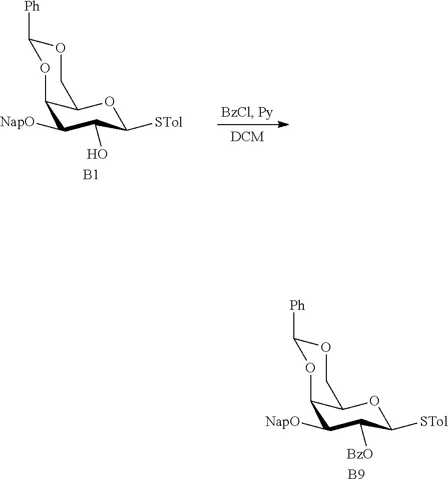

To a stirred solution of compound B1 (100 g, 0.1943 mol) in DCM (1 L) was sequentially added Pyridine (20.38 mL, 0.2526 mol), BzCl (28.98 mL, 0.2526 mol) drop wise at 0° C. followed by DMAP (14.22 g, 0.1165 mol), then the reaction mixture was allowed to stir rt for 14 h. After completion of the reaction was confirmed by TLC, the reaction mixture was poured into ice water. The organic layer was separated, washed with 5% cold citric acid solution (2×250 mL), brine solution (250 mL), dried over Na₂SO₄ and was evaporated under reduced pressure. The residue thus obtained was triturated with 5% ethyl acetate in hexane to afford compound B9 as a colourless solid (100.8 g, 84%).

### Synthesis of B10

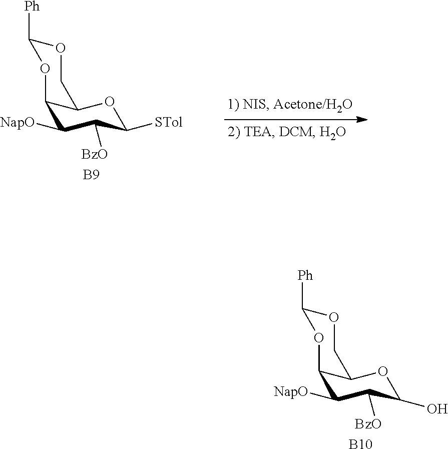

To the stirred solution of compound B9 (100.8 g, 0.1629 mol) in DCM:water (9:1, 2 L) was cooled to 5-10° C. and was added NIS (109.95 g, 0.4887 mol) portion-wise and was warmed to rt and stirred for 4 h. After the complete consumption of the SM was confirmed by TLC, the reaction mixture was quenched with 10% sodium thiosulfate solution, layers were separated. The organic layer was washed with brine, dried over anhydrous sodium sulphate, and evaporated under reduced pressure. The crude compound thus obtained was dissolved in DCM (2 L) and was cooled to 0° C. TEA (227 mL, 1.6291 mol) was added drop wise and the reaction mixture was allowed to stir at rt for 12 h. After the completion of the reaction was confirmed by TLC, the reaction mixture was evaporated under reduced pressure to afford the crude compound, which was purified by column chromatography over silica gel eluting with 35-40% of ethyl acetate in hexane to afford compound B10 as off-white solid (64.6 g, 77%).

### Synthesis of B11

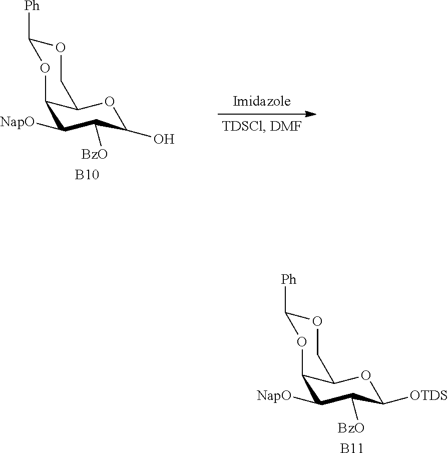

To a stirred solution of Compound B10 (64.6 g, 0.1260 mol) in DMF (517 mL) at rt, followed by imidazole (21.45 g, 0.315 mol), followed by TDS-CI (44.86 g, 0.2520 mol) drop wise at 0° C. . . . The reaction mixture was stirred at rt for 12 h. After the completion of the reaction was confirmed by TLC, the reaction mixture was poured into ice cold water and extracted with ethyl acetate (2×250 mL). The combined organic layer was washed with water, dried over Na₂SO₄. And was evaporated under reduced pressure. The crude compound thus obtained was purified by column chromatography over silica gel eluting with 12 to 14% EtOAc in Hexane to afford compound B11 as off-white solid (61.8 g, 75%).

### Synthesis of B12

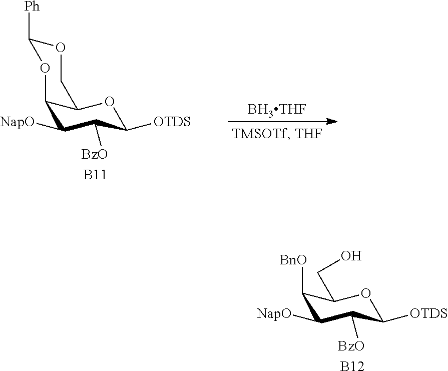

To a stirred solution of Compound B11 (61.8 g, 0.0944 mol) in THF (1.23 L) at rt was added BH₃ in THF (283 mL, 0.2834 mol) was drop wise added at −10° C., followed by TMSO-Tf (4.2 g, 0.0188 mol) and stirred at same temperature for 3 h. After the completion of the reaction was confirmed by TLC and LCMS, the reaction mixture was quenched with methanol, followed by TEA drop wise (effervescence observed), until the rm attains basic (PH ˜8) and stirred at RT for 14 h. The reaction mixture was evaporated under reduced pressure, the crude was diluted with DCM and washed with water, dried over Na₂SO₄, and evaporated under reduced pressure. The crude compound thus obtained was purified by column chromatography over silica gel eluting with 15 to 20% of ethyl acetate in hexane to afford compound B12 as colourless gummy liquid (47.73 g, 77%).

### Synthesis of B13

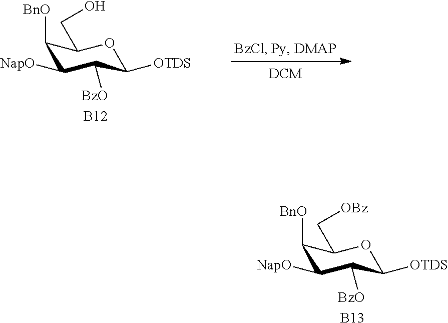

To the stirred solution of compound B12 (47.73 g, 0.0727 mol) in DCM (477 mL) at RT, was added by pyridine (11.51 g, 0.1455 mol) followed by BzCl (15.28 g, 0.1091 mol) drop wise added at 0° C. DMAP (0.88 g, 0.0072 mol) was added to the reaction mixture and was allowed to stir at RT for 12 h. After the completion of the reaction was confirmed by TLC, the reaction mixture was quenched with ice cold water, extracted with DCM (2×400 mL). The combined organic layer was washed with 5% acetic acid solution, dried over Na₂SO₄, and evaporated under reduced pressure. The crude compound thus obtained was purified by column chromatography over silica gel eluting with 10 to 15% of ethyl acetate in hexane to afford compound B13 as colourless solid (43.13 g, 78%).

### Synthesis of B14

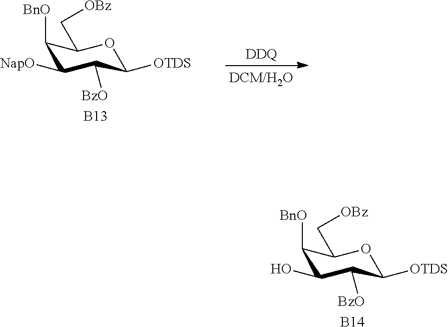

To a stirred solution of Compound B13 (43.13 g, 0.0566 mol) in DCM:Water (9:1, 862.6 mL) at RT, DDQ (38.59 g, 0.17 mol) was portion wise added at 0° C. The reaction mixture was allowed to stir at RT for 6 h. After the completion of the reaction was confirmed by TLC and LCMS, the reaction mixture was filtered over a celite bed. The filtrate was diluted with DCM and washed with sodium thiosulphate and saturated NaHCO₃ solution. The organic layer was dried over Na₂SO₄ and evaporated under reduced pressure. The crude compound thus obtained was purified by column chromatography over silica gel eluting with 10 to 15% of EtOAc in Hexane to afford B13 pale yellow gummy liquid (25.68 g, 73%). 1H NMR (400 MHz, Chloroform-d) δ 8.24-7.78 (m, 4H), 7.82-7.27 (m, 11H), 5.26 (dd, J=10.1, 7.6 Hz, 1H), 5.05-4.71 (m, 3H), 4.54 (dd, J=11.2, 7.3 Hz, 1H), 4.41 (dd, J=11.2, 5.7 Hz, 1H), 3.93 (dd, J=3.6, 1.2 Hz, 1H), 3.90-3.80 (m, 2H), 1.49 (hept, J=6.9 Hz, 1H), 0.80-0.60 (m, 12H), 0.14 (s, 3H), 0.08 (s, 3H).

### Synthesis of B16

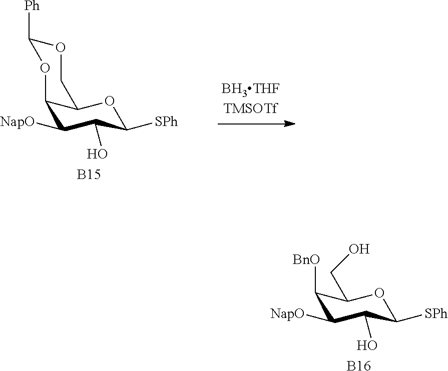

BH₃·THF (63 mL, 63.9 mmol) was added dropwise under nitrogen atmosphere at 0° C. to a stirred solution of compound B15 (see WO2019106201, page 147) (8.0 g, 15.9 mmol) in DCM (80 mL) followed by TMSOTf (1.5 mL, 7.9 mmol) was added at the same temperature over a period of 30 min. The reaction mixture was allowed to bring to room temperature and stirred at rt for 5 h. After completion of the reaction was confirmed by TLC, the reaction mixture was quenched with methanol (500 mL) and TEA (20 mL) and evaporated to dryness to get crude residue. The crude residue was quenched with NaHCO₃ (500 mL) and extracted with DCM (3×500 mL) and the combined organic layer was washed with water (2×500 mL), saturated brine (1×500 mL), dried over anhydrous sodium sulphate, filtered, and evaporated under reduced pressure. The crude compound thus obtained was washed with hexane to afford compound B16 as off-white solid (5.0 g, 62%).

### Synthesis of B17

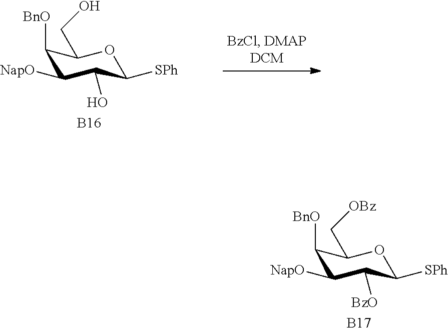

TEA (6.7 mL, 47.7 mmol) was added dropwise at 0° C. under nitrogen atmosphere to a stirred solution of compound B16 (3.0 g, 5.9 mmol) in DCM (30 mL) and BzCl (2.8 mL, 23.8 mmol) followed by DMAP (145 mg, 1.9 mmol) was added at the same temperature. The reaction mixture was allowed to bring to room temperature and stirred at rt for 13 h. After completion of the reaction was confirmed by TLC, the reaction mixture was quenched with ice water and extracted with DCM (3×500 mL) and separated the layers. The combined organic layer was washed with water (2×500 mL), saturated brine (1×500 mL), dried over anhydrous sodium sulphate and evaporated under reduced pressure. The crude compound thus obtained was further re-crystallized using hexanes to afford compound B17 as off-white solid (3.5 g, 83%).

### Synthesis of B18

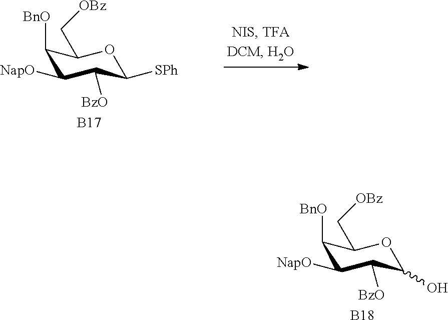

NIS (2.3 g, 105.0 mmol) was added at 0° C. under nitrogen atmosphere to a stirred solution of compound B17 (5.0 g, 7.0 mmol) in DCM:Water (25 mL: 5 mL) and TFA (0.27 mL, 3.5 mmol) was added dropwise at the same temperature over a period of 30 min. The reaction mixture was allowed to bring to room temperature and stirred at rt for 1 h. After completion of the reaction was confirmed by TLC, the reaction mixture was quenched with saturated NaHCO₃ (200 mL) and extracted with DCM (2×500 mL) and separated the layers. The combined organic layer was washed with water (2×500 mL), saturated brine (1×500 mL), dried over anhydrous sodium sulphate and evaporated under reduced pressure. The crude compound thus obtained was purified by column chromatography over silica gel (60-120 mesh) eluted with 10-15% EtOAc in hexane to afford compound B18 as pale-yellow liquid (3.4 g, 79%).

### Synthesis of B19

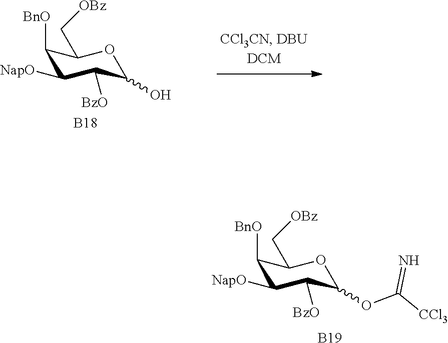

CCl₃CN (3.2 mL, 32.0 mmol) was added dropwise at −10° C. under nitrogen atmosphere to a stirred solution of compound B18 (2.0 g, 3.2 mmol) in DCM (20 mL), followed by DBU (0.049 mL, 0.032 mmol) was added dropwise at the same temperature over a period of 30 minutes. The reaction mixture was allowed to stir at 0° C. for 1 h. After completion of the reaction was confirmed by TLC, the reaction mixture was concentrated under reduced pressure. The crude residue thus obtained was purified by column chromatography over silica gel (60-120 mesh) eluted with 30-40% EtOAc in hexane to afford compound B19 as pale-yellow liquid (2.3 g, 95%).

### Synthesis of B20

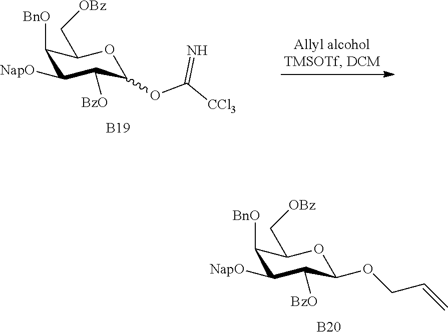

To a stirred solution of compound B19 (2.3 g, 3.0 mmol) in DCM (25 mL), 4 A° Molecular sieves were added and allowed to stir for 15 minutes. Then allyl alcohol (0.65 mL, 9.0 mmol) was added dropwise at 0° C. under nitrogen atmosphere followed by TMSOTf (0.05 mL, 0.3 mmol) dissolved in DCM (25 mL) also added in dropwise. The reaction mixture was allowed to bring to room temperature and stirred at rt for 10 h. After completion of the reaction was confirmed by TLC, the reaction mixture was quenched with saturated NaHCO₃ solution (50 mL) and extracted with DCM (3×50 mL) and separated the layers. The combined organic layer was washed with water (2×50 mL), saturated brine (1×50 mL), dried over anhydrous sodium sulphate and evaporated under reduced pressure. The crude thus obtained was further re-crystallized using hexanes to get compound B20 as off-white solid (800 mg, 42%). HRMS (ESI+) Calcd for C₄₁H₃₈O₈Na⁺ [M+Na]⁺ 681.2459, found 681.2512.

### Synthesis of B21

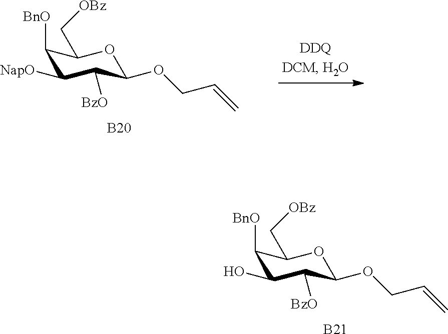

DDQ (0.69 g, 3.04 mmol) was added over 2.5 h to a stirred solution of compound B20 (800 mg, 1.21 mmol) in DCM:PBS buffer pH7.4 (12 mL: 12 mL) and allowed to stir at rt for 4 h. After completion of the reaction was confirmed by TLC, the reaction mixture was filtered using celite bed and the filtrate was washed with saturated NaHCO₃ solution (50 mL) and extracted with DCM (2×50 mL) and separated the layers. The combined organic layer was washed with water (2×50 mL), saturated brine (1×50 mL), dried over anhydrous sodium sulphate and evaporated under reduced pressure to get crude compound. The crude compound thus obtained was purified by column chromatography over silica gel eluted with EtOAc in hexane to afford compound B21 as off-white solid (0.57 g, 91%). HRMS (ESI+) Calcd for C₄₁H₃₈O₈Na⁺ [M+Na]⁺ 541.1833, found 541.1883.

### Synthesis of thexyldimethylsilyl 4,6-di-O-benzoyl-2-O-benzyl-3-O-levulinoyl-α-D-galactopyranosyl-(1→3)-2,6-di-O-benzoyl-4-O-benzyl-β-D-galactopyranoside B22

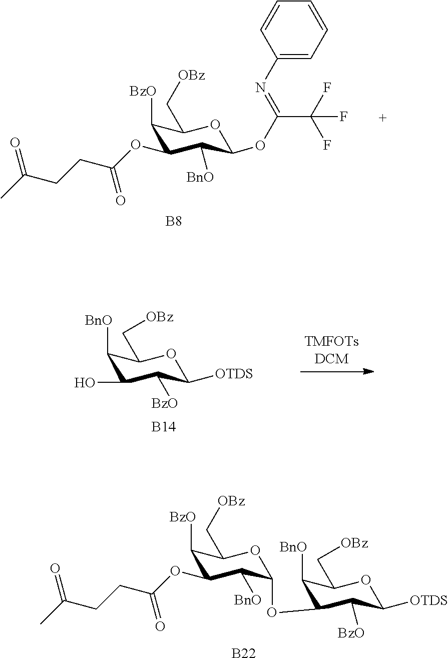

A mixture of donor B8 (11.91 g, 15.93 mmol) and acceptor B14 (8.24 g, 13.27 mmol) was dissolved in anhydrous Toluene (3×50 mL), dried azeotropically and the residue dried at high vacuum for 20 min. The residue was dissolved in anhydrous DCM (180 mL), microwave-dried MS 4 Å added and the resulting yellow solution stirred at RT for 1 h. The solution was then cooled to 0° C. and TMSOTf (0.48 mL, 2.65 mmol) added dropwise. After stirring the reaction mixture at 0° C. for 40 min, TLC reaction control indicated completion of the reaction. The reaction mixture was allowed to reach RT, quenched by addition of TEA (0.37 mL) stirred for additional 5 min and then filtered over a cotton-plug covered with sea sand and plug washed with DCM (3×100 mL). The filtrate was concentrated under reduced pressure to get the crude. Purified by flash column chromatography on silica column using EA/Cyclohexane as eluents to yield the desired compound B22 as white foam (13.46 g, 86%). ¹H NMR (400 MHZ, CDCl₃) δ 8.17-7.84 (m, 8H), 7.74-7.12 (m, 22H), 5.68 (dd, J=10.2, 7.5 Hz, 1H), 5.51-5.36 (m, 2H), 5.23-5.09 (m, 2H), 4.89-4.57 (m, 4H), 4.46 (dd, J=11.3, 7.2 Hz, 1H), 4.38-4.26 (m, 2H), 4.11-3.98 (m, 3H), 3.96-3.87 (m, 2H), 3.71 (t, J=6.3 Hz, 1H), 2.97-2.29 (m, 4H), 2.10 (s, 3H), 1.53-1.43 (m, 1H), 0.82-0.60 (m, 12H), 0.13 (s, 3H), 0.02 (s, 3H). HRMS-QTOF Calcd for C₆₇H₇₄NaO₁₇Si⁺ [M+Na]⁺ 1201.4587, found 1201.4351.

### Synthesis of allyl 4,6-di-O-benzoyl-2-O-benzyl-3-O-levulinoyl-α-D-galactopyranosyl-(1→3)-2,6-di-O-benzoyl-4-O-benzyl-β-D-galactopyranoside B23

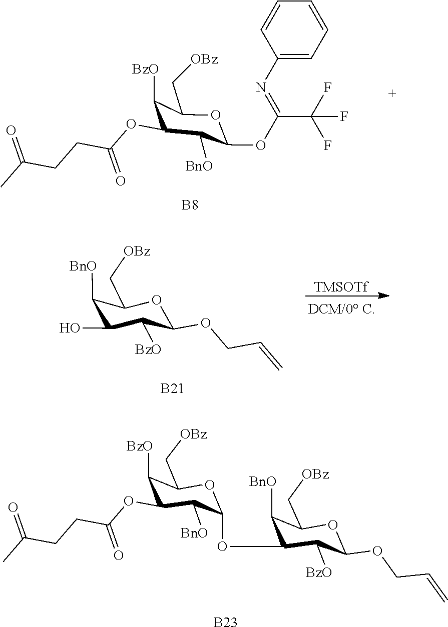

A mixture of donor B8 (39.8 g, 53.2 mmol) and acceptor B21 (23 g, 44.4 mmol) were taken in 1 L RBF, added dried 4 Å molecular sieves (MS) and anhydrous DCM (590 mL). It was stirred at rt for 45 min and then cooled to 0° C. TMSOTf (1.6 mL, 8.87 mmol) was added to the reaction mixture and stirred for 30 min. Then it was slowly warmed to 10° C. for 0.5 h and then stirred at rt for 1 h. The reaction was quenched at 5° C. by the addition of TEA (1.2 mL, 8.87 mmol). The crude product was purified by a flash chromatography system using EtOAc/Cy gradient system. The collected fraction was concentrated in vacuum and dried under high vacuum for 16 h to afford the product B23 as off-white fluffy solid (45.1 g, 94%). ¹H NMR (400 MHZ, CDCl₃) δ 8.23-7.81 (m, 8H), 7.80-6.81 (m, 22H), 5.88-5.63 (m, 2H), 5.53-5.33 (m, 2H), 5.25-4.97 (m, 4H), 4.84-4.46 (m, 5H), 4.45-4.26 (m, 3H), 4.22-3.85 (m, 6H), 3.78-3.67 (m, 1H), 2.94-2.24 (m, 4H), 2.08 (s, 3H). HRMS-QTOF Calcd for C₆₂H₆₀NaO₁₇⁺ [M+Na]⁺ 1099.3723, found 1099.3817.

### Synthesis of Disaccharide Acceptor B24

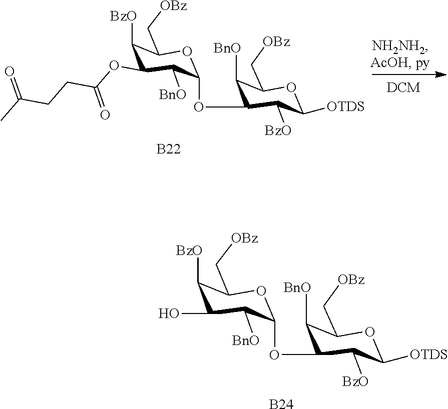

B22 (13.46 g, 11.41 mmol) was dissolved in DCM (285 mL) and to this stirred colorless solution at RT, a mixture of hydrazine monohydrate (1.42 mL, 45.70 mmol), pyridine (36.9 mL, 457 mmol) and AcOH (24.8 mL, 434 mmol) was added and stirred for 2 h. The reaction was quenched by addition of acetone (50 mL) and stirred for additional 45 min. The reaction mixture was concentrated under reduced pressure to yield the crude as an oily residue, which was purified by automated flash column chromatography using EA/cyclohexane to yield the desired compound B24 as colorless foam (12.16 g, 99%). HRMS-QTOF Calcd for C₆₂H₇₂NO₁₅Si⁺ [M+NH₄]⁺1098.4666, found 1098.4429.

### Synthesis of Disaccharide Acceptor B25

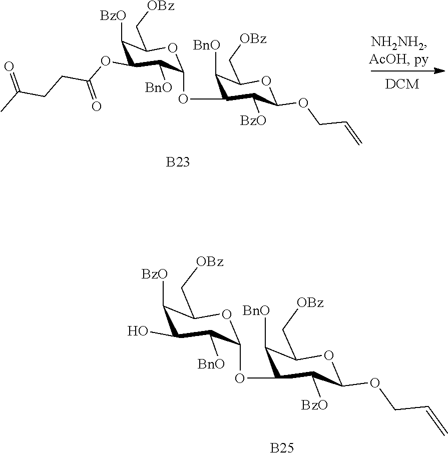

To a solution of B23 (15 g, 13.93 mmol) in DCM, a solution of hydrazine hydrate (1.7 mL, 55.7 mmol) dissolved in AcOH (30.3 mL, 529 mmol) and py (45 mL, 557 mmol) was added. The resulting reaction mixture was stirred at rt for 2 h. The reaction was quenched by the addition of acetone (50 mL) and the solvent removed under vacuum to obtain the crude product. The crude product was purified by automated flash chromatography using EtOAc/Cy gradient system as the eluent. Concentration of solvent from test tubes containing the product in vacuum resulted in white solid B25 (12.93 g, 95%). HRMS-QTOF Calcd for C₅₇H₅₄NaO₁₅⁺ [M+Na]⁺ 1001.3355, found 1001.3309.

### Synthesis of Disaccharide Hemiacetal B26 from B22

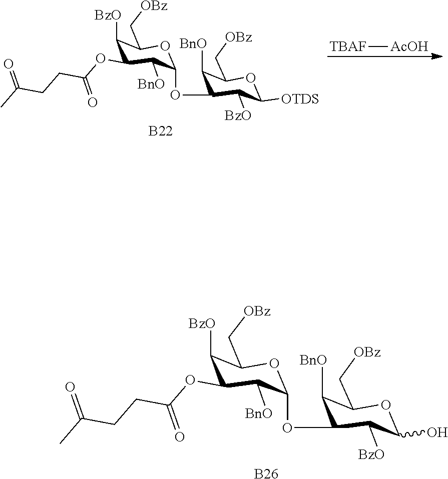

B22 (16.14 g, 13.68 mmol) dried azeotropically using dry toluene and the residue dried at high vacuum for 30 min. Then the material was dissolved in anhydrous DCM (140 mL) and added AcOH (12.53 mL, 219 mmol) to it and stirred 5 min. Added 1M solution of TBAF in anhydrous THF (205 mL, 205 mmol) to the RM and stirred the reaction mixture at RT for 24 h, till the completion. The reaction was quenched by addition of water (100 mL), the phases were separated, and the aq. phase extracted with DCM (2×100 mL) The combined org. phases were washed with sat. aq. NaHCO₃ (200 mL) and brine (200 mL), dried over anhydrous Na₂SO₄, filtered and concentrated in vacuum to yield a crude colorless oil. The crude was purified by automated flash column chromatography using DCM/cyclohexane as eluents and fractions containing product were concentrated to yield the desired product as white foam B26 (14.11 g, 99%). HRMS-QTOF Calcd for C₅₉H₆₀NO₁₇⁺ [M+NH₄]⁺ 1054.3856, found 1054.3628.

### Synthesis of Disaccharide Hemiacetal B26 from B23

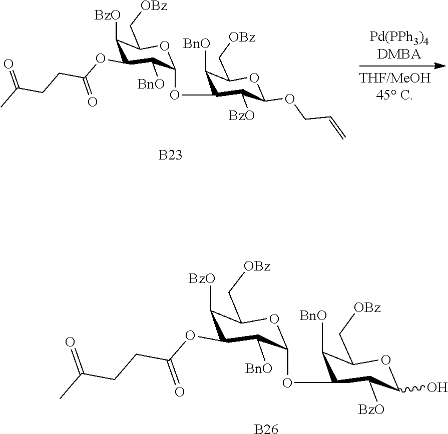

B23 (26 g, 24.1 mmol) and N,N-Dimethyl barbituric acid (DMBA) (9.42 g, 60.3 mmol) were taken in MeOH (290 mL) under nitrogen (suspension) and added THF (190 mL) to it (became clear solution), heated to 45° C. for 2 min. The RM was degasified and filled with N₂ three times followed by the addition of 5 mol % Pd(PPh₃)₄ (1.40 g, 1.2 mmol). Then, the RM was again degasified, and the reaction vessel filled with N₂ (twice). Continued heating at 50° C. of yellow colored solution for 16 h. RM was cooled to rt and diluted with EtOAc (500 mL) and washed with NaHCO₃ solution (200 mL), brine (150 mL), dried over Na₂SO₄, filtered and concentrated in vacuum. Purification by a flash chromatography system using EtOAC/Cy gradient system yielded product B26 as yellowish fluffy solid (21.52 g, 86%). HRMS-QTOF Calcd for C₅₉H₅₆NaO₁₇⁺ [M+Na]⁺ 1059.3410, found 1059.3384.

### Synthesis of Disaccharide Donor B27

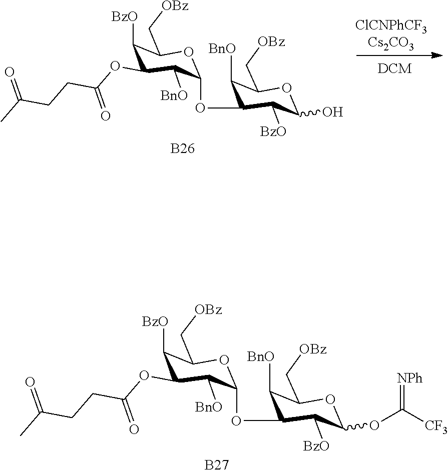

B26 (21.52 g, 20.75 mmol) was dissolved in anhydrous DCM (260 mL). Cs₂CO₃ (13.52 g, 41.5 mmol) and 2,2,2-trifluoro-N-phenylacetimidoyl chloride (9.9 mL, 62.3 mmol) were added to the solution. The reaction mixture was stirred at rt overnight. The reaction mixture was filtrated through a Celite pad. The pad was washed with DCM (250 mL), the filtrate concentrated under reduced pressure, co-evaporated with toluene and dried under high vacuum to give pale yellow solid B27 (25 g, 100%). HRMS-QTOF Calcd for C₆₇H₆₀F₃NNaO₁₇⁺ [M+Na]⁺ 1230.3706, found 1230.3672.

### Synthesis of thexyldimethylsilyl 4,6-di-O-benzoyl-2-O-benzyl-3-O-levulinoyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranoside B28

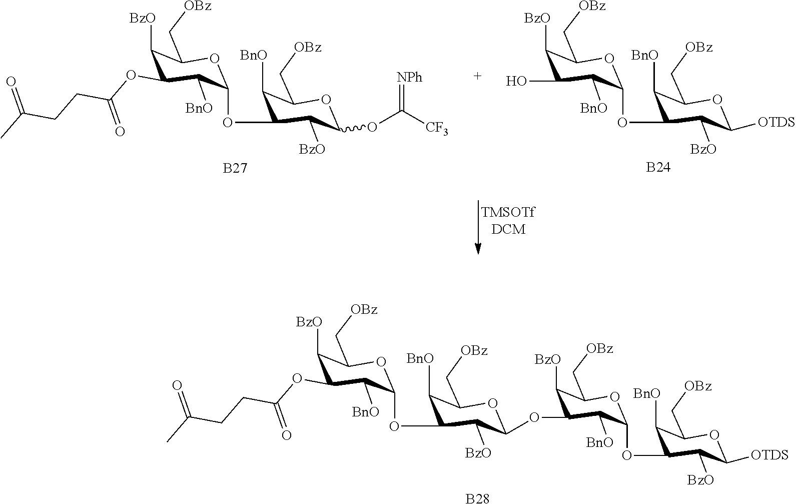

Both the acceptor B24 (6.62 g, 6.12 mmol) and the donor B27 (8.51 g, 7.04 mmol) were taken in RBF and dried azeotropically using dry toluene in the vacuum. Mixture was taken in anhydrous DCM (120 mL) at rt, added 4 Å molecular sieves to it and stirred at for 30 min under N₂ atmosphere. Cooled the RM to −2 deg using ice water bath and added TMSOTf (0.22 mL, 1.224 mmol) to the RM and stirred the RM at 5 deg for 20 mins. RM was then allowed to warm slowly to room temp over one hr. TLC analysis was carried out to monitor the completion of the reaction. RM was quenched by addition of TEA (0.2 mL) stirred for additional 5 min and then filtered over a cotton-plug covered with sea sand and plug washed with DCM (3×100 mL) and concentrated in vacuum. Column purification of crude product was done on silica using EA/cyclohexane on Biotage®. Fractions containing product were evaporated and dried in vacuum to yield the desired compound as white foam B28 (10.95 g, 85%). HRMS-QTOF Calcd for C₁₂₁H₁₂₂NaO₃₁Si⁺ [M+Na]⁺ 2121.7632, found 2121.6391.

### Synthesis of allyl 4,6-di-O-benzoyl-2-O-benzyl-3-O-levulinoyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranoside B29

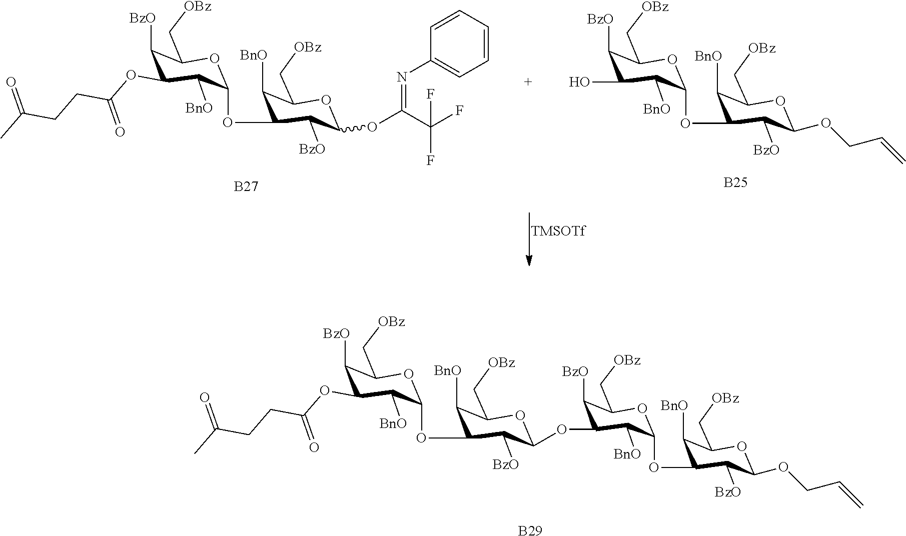

B27 (19.55 g, 16.18 mmol) and B25 (13.2 g, 13.48 mmol) were taken in a RBF, added dried 4 Å molecular sieves (MS) and anhydrous DCM (270 mL). It was stirred at rt for 45 min and then cooled to 0° C. TMSOTf (0.49 mL, 2.70 mmol) was added to the reaction mixture and stirred for 30 min. Then it was slowly warmed to 10° C. for 0.5 h and then stirred at rt for 1 h. The reaction was quenched at 5° C. by the addition of TEA (0.38 mL, 2.70 mmol). The crude product was purified by a flash chromatography system using EtOAc/Cy Gradient system. The collected fraction was concentrated in vacuum and dried under high vacuum for 16 h to afford the product B29 as off-white fluffy solid (25 g, 93%). HRMS-QTOF Calcd for C₁₁₆H₁₀₈NaO₃₁⁺ [M+Na]⁺ 2019.6767, found 2019.6693.

### Synthesis of Tetrasaccharide Hemiacetal B30 from B28

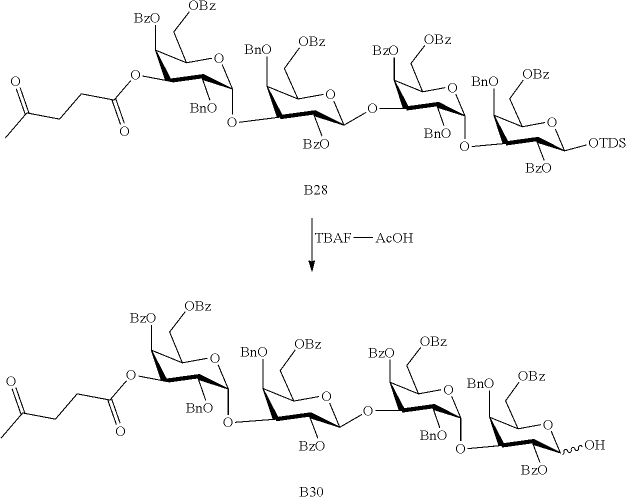

Substrate B28 (13 g, 6.19 mmol) dried azeotropically using dry toluene and the residue dried at high vacuum for 30 min. Then the material was dissolved in anhydrous DCM (60 mL) and added AcOH (5.67 mL, 99 mmol) to it and stirred 5 min. Added 1M solution of TBAF in anhydrous THF (93 mL, 93 mmol) to the RM and stirred the reaction mixture at RT for 24 h, till the completion. The reaction was quenched by addition of water (50 mL) and vortexed for 5 min. The phases were separated, and the aq. phase was extracted with DCM (2×50 mL). The combined org. phases were washed with sat. aq. NaHCO₃ (50 ml×2) and brine (50 mL). The combined org. phase dried over anhydrous Na₂SO₄, filtered and concentrated in vacuum to yield a crude colorless oil. The crude was purified by automated flash column chromatography using EA/Cy as eluents to yield the desired product as white foam B30 (11.3 g, 93%). HRMS-QTOF Calcd for C₁₁₃H₁₀₈NO₃₁⁺ [M+NH₄]⁺ 1974.6900, found 1974.7003.

### Synthesis of Tetrasaccharide Hemiacetal B30 from B29

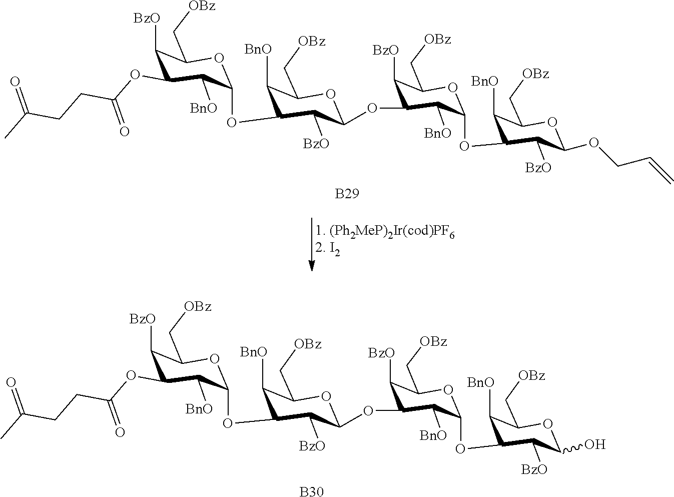

(1,5-Cyclooctadiene) (pyridine) (tricyclohexylphosphine)-Ir(I)]PF₆ (0.56 g, 0.661 mmol) was dissolved in THF (65 mL) and N₂ was bubbled through the solution for two min at rt while the red colored catalyst dissolved. The solution was then purged with H₂ for 5 min, by which time the red solution changed to colorless and the solution was stirred for 15 min under hydrogen. The solution of the active catalyst was then added to a solution of the B29 (13.2 g, 6.61 mmol) in THF (130 mL) under N₂ via a syringe and stirred for 16 h at rt. The reaction mixture was quenched with sat. aq. NaHCO₃ solution (100 mL) and extracted with EtOAc (3×100 mL). Combined organic layers were washed with brine (100 mL), dried over Na₂SO₄, filtered and evaporated to get the allyl isomerized compound (isomerization confirmed by 1H NMR). The vinyl substrate was then taken up in a mixture of THF:H₂O (2:1, 130 mL:65 mL)) and |2 (3.35 g, 13.21 mmol) was added at rt. The brown colored solution was stirred for 2 h before quenching with 10% solution of Na₂S₂O₃ solution (250 mL). The aqueous phase was extracted with EtOAc (3×100 mL) and the combined organic layers were dried over Na₂SO₄, filtered and the solvent evaporated. Flash column chromatography (EtOAc/Cy gradient system) afforded the product as yellow solid B30 (12 g, 93%). HRMS-QTOF Calcd for C₁₁₃H₁₀₄NaO₃₁⁺ [M+Na]⁺ 1979.6454, found 1979.6497.

### Synthesis Tetrasaccharide Donor B31

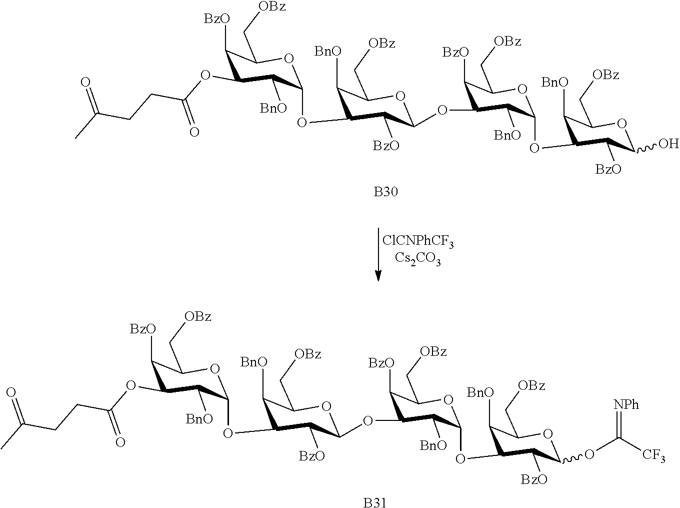

B30 (7.4 g, 3.78 mmol) was dissolved in anhydrous DCM (47 mL). Cs₂CO₃ (2.46 g, 7.56 mmol) and 2,2,2-trifluoro-N-phenylacetimidoyl chloride (1.80 mL, 11.34 mmol) were added to the solution. The reaction mixture was stirred at rt overnight. The reaction mixture was filtrated through a Celite pad. The pad was washed with DCM (150 ml×2), the filtrate concentrated under reduced pressure, co-evaporated with toluene and dried under high vacuum to give pale yellow solid B31 (8.0 g, 99%). HRMS-QTOF Calcd for C₁₂₁H₁₀₈F₃NNaO₃₁⁺ [M+Na]⁺ 2150.6750, found 2150.6774.

### Synthesis of Tetrasaccharide Acceptor B32

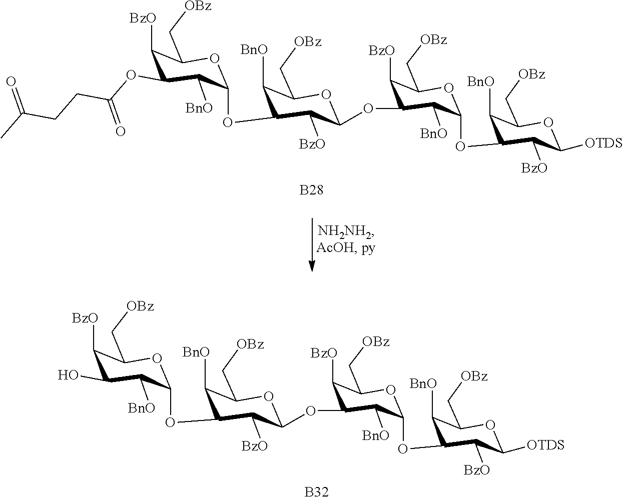

To a solution of the B28 (3 g, 1.428 mmol) in DCM (35 mL), a solution of hydrazine hydrate (0.18 mL, 5.71 mmol) dissolved in acetic acid (3.11 mL, 54.3 mmol) and pyridine (4.62 mL, 57.1 mmol) was added. The resulting reaction mixture was stirred at rt for 2 h. The reaction was quenched by the addition of acetone (20 mL) and the solvent removed under vacuum to obtain the crude product. The crude product was purified by automated flash chromatography using EtOAc/Cy gradient system as the eluent. Concentration of solvent from test tubes containing the product (based on TLC) in vacuum resulted in white solid B32 (2.5 g, 87%). HRMS-QTOF Calcd for C₁₁₆H₁₁₆NaO₂₉Si⁺ [M+Na]⁺ 2023.7264, found 2023.6054.

### Synthesis of Tetrasaccharide Acceptor B33

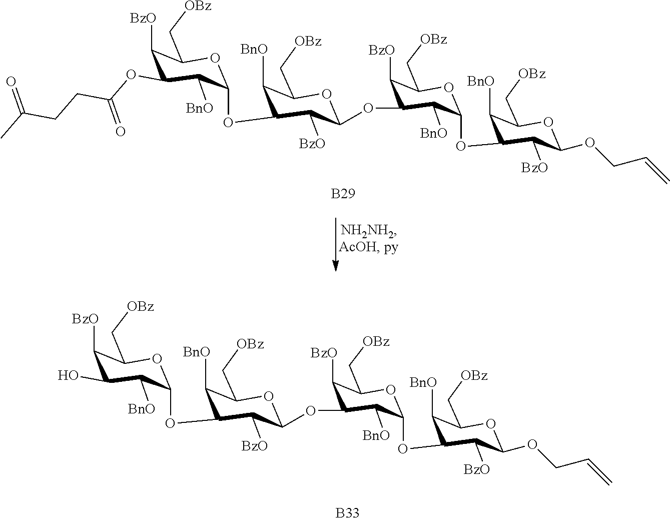

To a solution of the B29 (11 g, 5.51 mmol) in DCM (140 mL), a solution of hydrazine hydrate (0.68 mL, 22.02 mmol) dissolved in acetic acid (12.0 mL, 209 mmol) and pyridine (17.81 mL, 220 mmol) was added. The resulting reaction mixture was stirred at rt for 2 h. The reaction was quenched by the addition of acetone (20 mL) and the solvent removed under vacuum to obtain the crude product. The crude product was purified by automated flash chromatography using EtOAc/Cy gradient system as the eluent. Concentration of solvent from test tubes containing the product (based on TLC) in vacuum resulted in white solid B33 (10.1 g, 97%). HRMS-QTOF Calcd for C₁₁₁H₁₀₂NaO₂₉⁺ [M+Na]⁺ 1921.6399, found 1921.6449.

### Synthesis of Octasaccharide B34

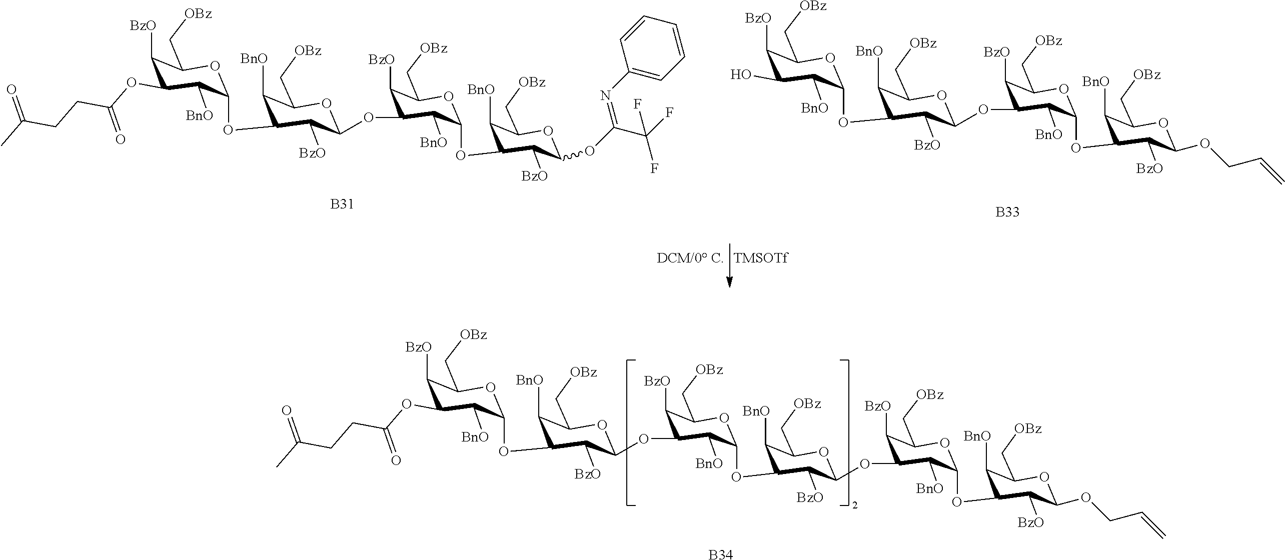

B31 (8.07 g, 3.79 mmol) and B33 (6.0 g, 3.16 mmol) were taken in a RBF, added dried 4 Å molecular sieves (MS) and anhydrous. DCM (65 mL). It was stirred at rt for 45 min and then cooled to 0° C. TMS-OTf (0.12 mL, 0.63 mmol) was added to the reaction mixture and stirred for 30 min. Then it was slowly warmed to 10° C. for 0.5 h and then stirred at rt for 1 h. The reaction was quenched at 5° C. by the addition of triethylamine (0.09 mL, 0.63 mmol). The crude product was purified by a flash chromatography system using EtOAc/Cy gradient system. The collected fraction was concentrated in vacuum and dried under high vacuum for 16 h to afford the product B34 as off-white fluffy solid (9.96 g, 82%). MALDI-TOF Calcd for C₂₂₄H₂₀₄NaO₅₉⁺ [M+Na]⁺ 3860.2855, found 3861.337.

### Synthesis of Octasaccharide Acceptor B35

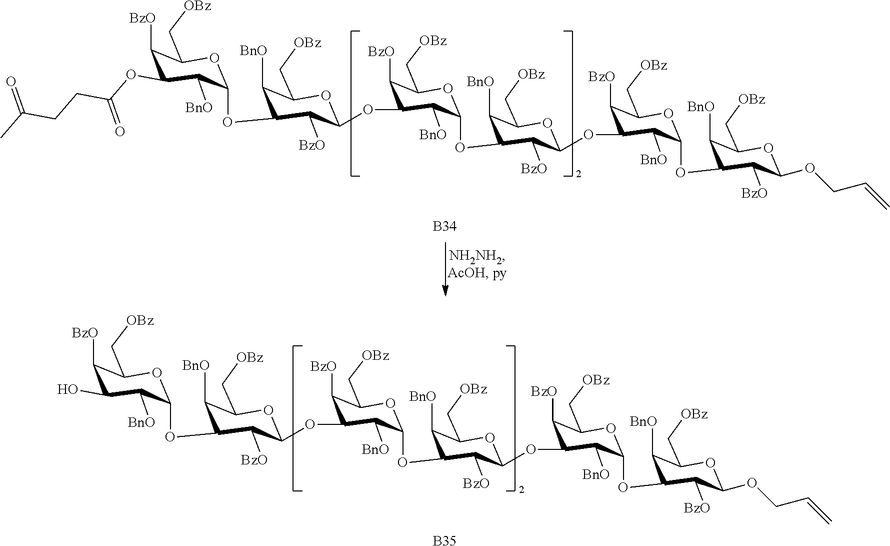

To a solution of B34 (12 g, 3.12 mmol) in DCM (78 mL), a solution of NH₂NH₂ (0.40 mL, 12.50 mmol) dissolved in AcOH (6.80 mL, 119 mmol) and py (10.10 mL, 125 mmol) was added. The resulting reaction mixture was stirred at rt for 2 h. The reaction was quenched by the addition of acetone (10 mL) and the solvent removed under vacuum to obtain the crude product. The crude product was purified by automated flash chromatography using EtOAc/Cy gradient system as the eluent. Concentration of solvent from test tubes containing the product (based on TLC) in vacuum resulted in white solid B35 (10.52 g, 90%). MALDI-TOF Calcd for C₂₁₉H₁₉₈NaO₅₇⁺ [M+Na]⁺ 3762.2487, found 3764.559.

### Synthesis of Octasaccharide Hemiacetal B36

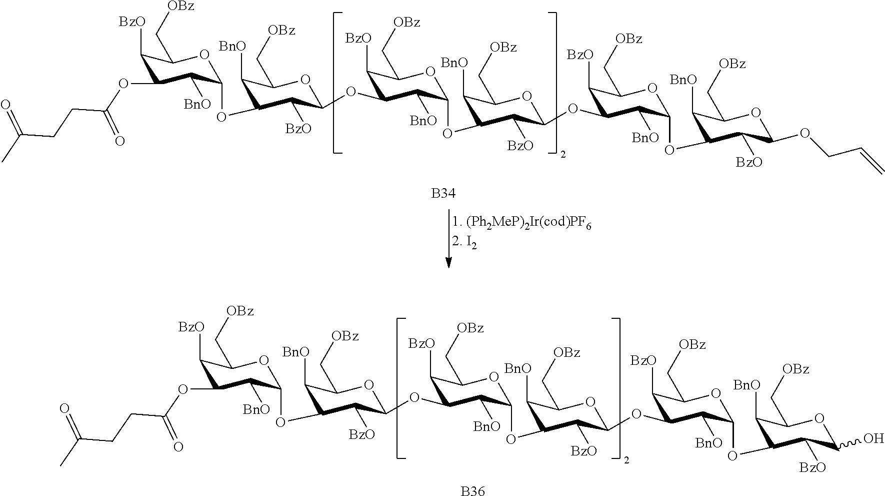

(1,5-Cyclooctadiene)(pyridine)(tricyclohexylphosphine)-Ir(I)]PF₆ (0.22 g, 0.258 mmol) was dissolved in THF (25 mL) and N₂ was bubbled through the solution for two min at rt while the red colored catalyst dissolved. The solution was then purged with Hz for two min, by which time the red solution changed to colorless, and the solution was stirred for 15 min under hydrogen. The solution of the active catalyst was then added to a solution of the B34 (9.9 g, 2.58 mmol) in THF (50 mL) under N₂ via a syringe and stirred for 16 h at rt. The reaction mixture was quenched with sat. aq. NaHCO₃ solution (100 mL) and extracted with EtOAc (3×100 mL). Combined organic layers were washed with brine (100 mL), dried over Na₂SO₄, filtered, and evaporated to get the allyl isomerized compound (isomerization confirmed by ¹H NMR). The vinyl substrate was then taken up in a mixture of THF:H₂O (2:1, 50 mL:25 mL)) and |2 (1.31 g, 5.16 mmol) was added at rt. The brown colored solution was stirred for 2 h before quenching with 10% solution of Na₂S₂O₃ solution (100 mL). The aqueous phase was extracted with EtOAc (3×50 mL) and the combined organic layers were dried over Na₂SO₄, filtered and the solvent evaporated. Flash column chromatography (EtOAc/Cy gradient system) afforded the product as yellow solid B36 (8.0 g, 82%). MALDI-TOF Calcd for C₂₂₁H₂₀₀NaO₅₉⁺ [M+Na]⁺ 3820.2542, found 3821.524.

### Synthesis of Octasaccharide Donor B37

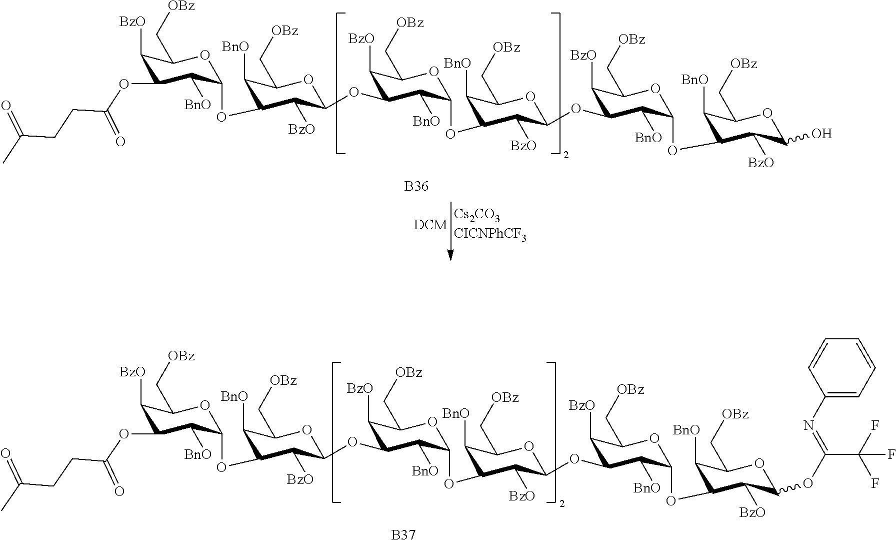

B36 (5 g, 1.316 mmol) was dissolved in anhydrous DCM (17 mL). Cs₂CO₃ (0.86 g, 2.63 mmol) and 2,2,2-trifluoro-N-phenylacetimidoyl chloride (0.82 g, 3.95 mmol) were added to the solution. The reaction mixture was stirred at rt overnight. The reaction mixture was filtered through a Celite pad. The pad was washed with DCM (250 mL), the filtrate concentrated under reduced pressure and dried azeotropically using toluene. The residue was dried under high vacuum for 16 h to afford the product B37 as off-white fluffy solid (5.1 g, 98%).

### Synthesis of Dodecasaccharide B38 Using (8+4) Approach

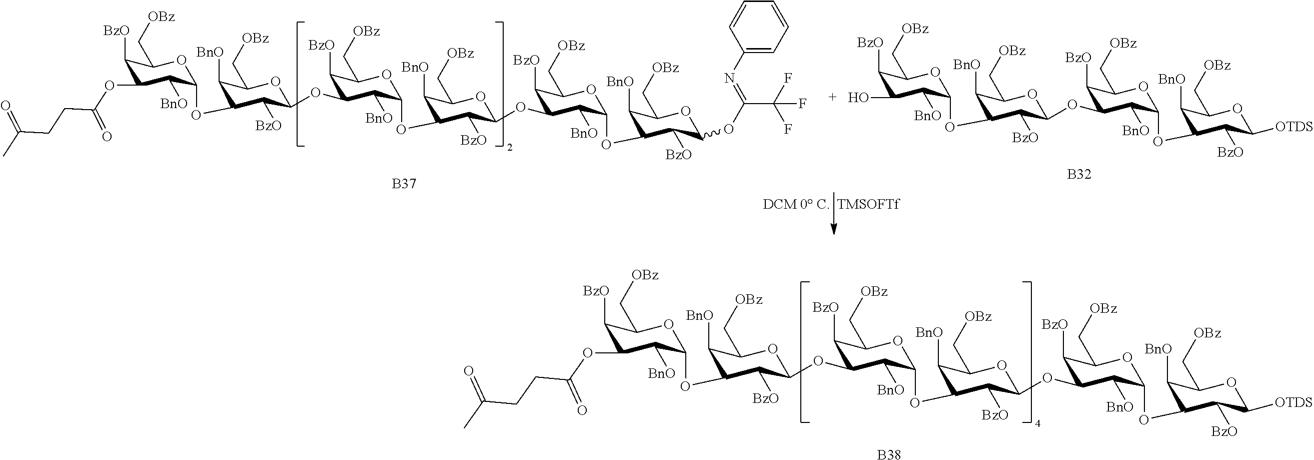

B37 (1.6 g, 0.403 mmol) and B32 (0.81 g, 0.403 mmol) were taken in a RBF, added dried 4 Å MS and anhydrous DCM (6 mL). It was stirred at rt for 45 min and then cooled to 0° C. TMSOTf (15 μL, 0.081 mmol) was added to the reaction mixture and stirred for 30 min. Then it was slowly warmed to 10° C. for 0.5 h and then stirred at rt for 1 h. The reaction was quenched at 5° C. by the addition sat. aq. NaHCO₃ solution (25) and extracted with DCM (3×50 mL). Combined organic layers were washed with brine (100 mL), dried over Na₂SO₄, filtered, and evaporated to get crude. The crude product was purified by flash chromatography using EtOAc/Cy gradient system. The collected fraction was concentrated in vacuum and dried under high vacuum for 16 h to afford the product B38 as white fluffy solid (1.63 g, 70%).

### Synthesis of Dodecasaccharide B39 Using (4+8) Approach

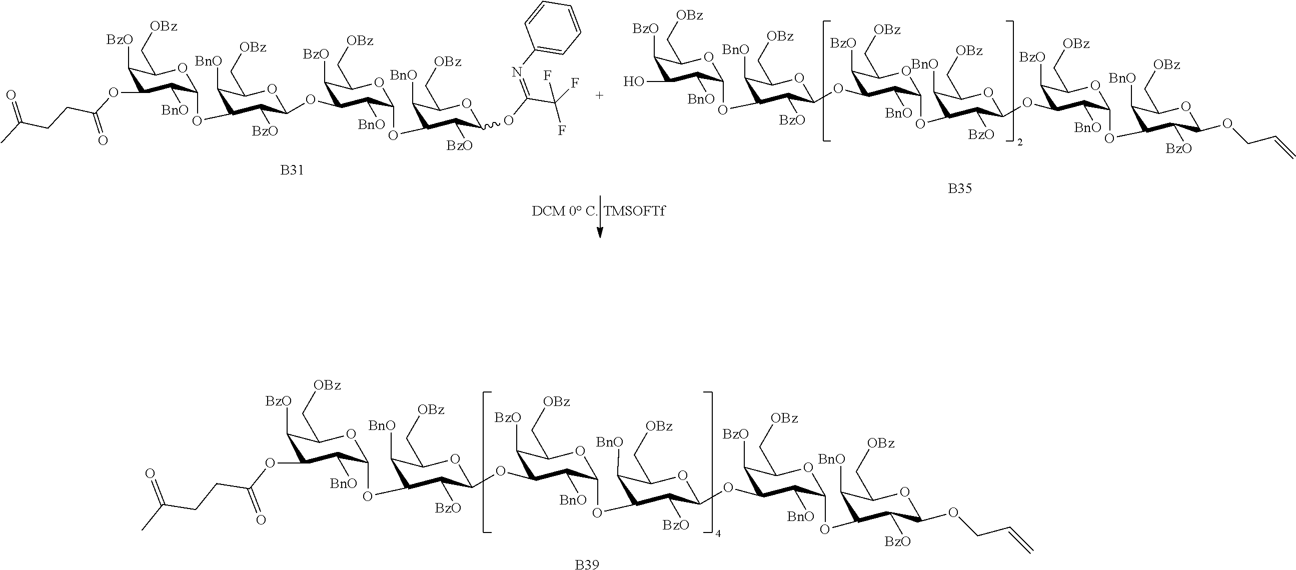

B31 (7.17 g, 3.37 mmol) and B35 (10.5 g, 2.81 mmol) were taken in a RBF, added dried 4 Å MS and anhydrous DCM (56 mL). It was stirred at rt for 45 min and then cooled to 0° C. TMSOTf (0.10 mL, 0.56 mmol) was added to the reaction mixture and stirred for 30 min. Then it was slowly warmed to 10° C. for 0.5 h and then stirred at rt for 1 h. The reaction was quenched at 5° C. by the addition of TEA (0.08 mL, 0.56 mmol). The crude product was purified by flash chromatography using EtOAc/Cy gradient system. The collected fraction was concentrated in vacuum and dried under high vacuum for 16 h to afford the product B39 as white fluffy solid (12.10 g, 76%). MALDI-TOF Calcd for C₃₃₂H₃₀₀KO₈₇⁺ [M+K]⁺ 5716.8682, found 5714.291.

### Synthesis of Dodecasaccharide Hemiacetal B40 from B38

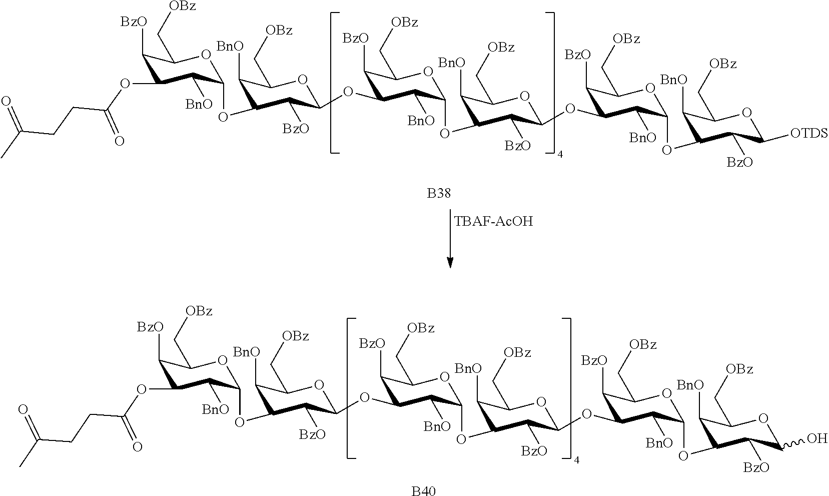

B38 (1.63 g, 0.282 mmol) dried azeotropically using dry toluene and the residue dried at high vacuum for 30 min. Then the material was dissolved in anhydrous DCM (3 mL) and added AcOH (0.25 mL, 4.37 mmol) to it and stirred 5 min. Added 1M solution of TBAF in anhydrous THF (4.23 mL, 4.23 mmol) to the RM and stirred the reaction mixture at RT for 24 h, till the completion. The reaction was quenched by addition of water (100 mL) and extracted with DCM (2×50 mL). The combined org. phases were washed with sat. aq. NaHCO₃ (200 mL) and brine (200 mL), dried over anhydrous Na₂SO₄, filtered and concentrated in vacuum to yield a crude colorless oil. The crude was purified by automated flash column chromatography using EtOAc/cyclohexane as eluents and fractions containing product were concentrated to yield the desired product as white foam B40 (1.26 g, 79%).

### Synthesis of Dodecasaccharide Hemiacetal B40 from B39

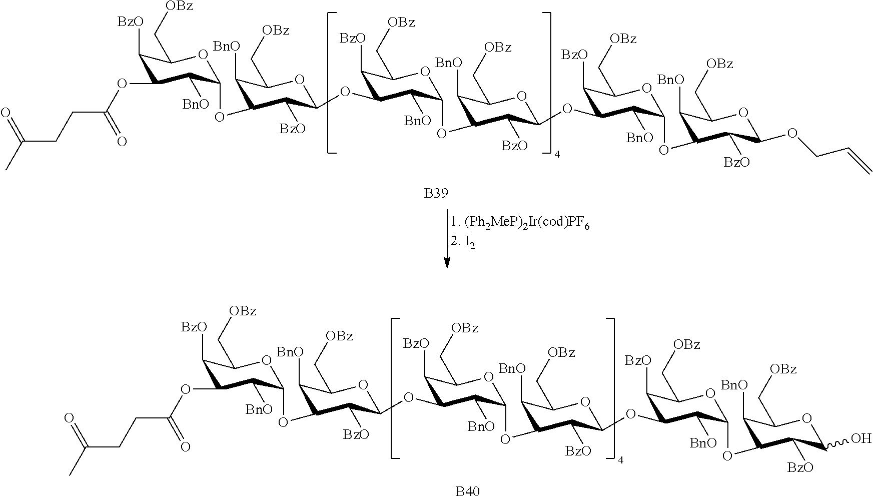

(1,5-Cyclooctadiene) (pyridine) (tricyclohexylphosphine)-Ir(I)]PF₆ (0.12 g, 0.141 mmol) was dissolved in THF (15 mL) and N₂ was bubbled through the solution for two min at rt while the red colored catalyst dissolved. The solution was then purged with H₂ for two min, by which time the red solution changed to colorless and the solution was stirred for 15 min under hydrogen. The solution of the active catalyst was then added to a solution of the B39 (8.0 g, 1.408 mmol) in THF (30 mL) under N₂ via a syringe and stirred for 16 h at rt. The reaction mixture was quenched with sat. aq. NaHCO₃ solution (50 mL) and extracted with EtOAc (3×50 mL). Combined organic layers were washed with brine (50 mL), dried over Na₂SO₄, filtered and evaporated to get the allyl isomerized compound (isomerization confirmed by 1H NMR). The vinyl substrate was then taken up in a mixture of THF:H₂O (2:1, 28 mL: 14 mL)) and 12 (0.71 g, 2.82 mmol) was added at rt. The brown colored solution was stirred for 2 h before quenching with 10% solution of Na₂S₂O₃ solution (50 mL). The aqueous phase was extracted with EtOAc (3×5 mL) and the combined organic layers were dried over Na₂SO₄, filtered and the solvent evaporated. Flash column chromatography (EtOAc/Cy gradient system) afforded the product as yellow solid B40 (5.7 g, 72%). MALDI-TOF Calcd for C₃₂₉H₂₉₆NaO₈₇⁺ [M+Na]⁺ 5660.8630, found 5665.367.

### Synthesis of Dodecasaccharide Donor B41

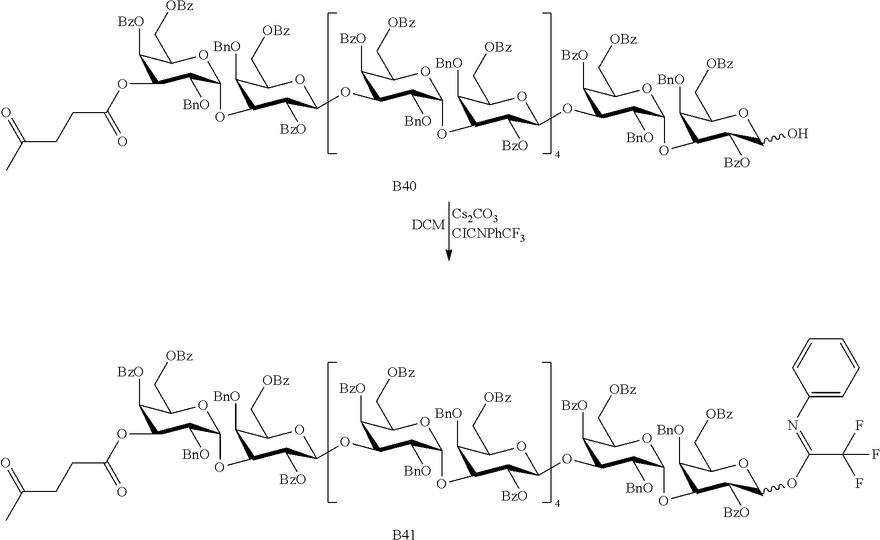

B40 (2.0 g, 0.354 mmol) was dissolved in anhydrous DCM (5 mL). Cs₂CO₃ (0.231 g, 0.709 mmol) and 2,2,2-trifluoro-N-phenylacetimidoyl chloride (0.17 mL, 1.063 mmol) were added to the solution. The reaction mixture was stirred at rt overnight. The reaction mixture was filtered through a Celite pad. The pad was washed with DCM (50 ml×2), the filtrate concentrated under reduced pressure. The collected fraction was concentrated and dried azeotropically using toluene under vacuum to afford the product B41 as off-white fluffy solid (2.0 g, 99%).

### Synthesis of 5-azido-pentyl 4,6-di-O-benzoyl-2-O-benzyl-3-O-levulinoyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)]-6-O-benzoyl-2,4-di-O-benzyl-α-D-galactopyranosyl-(1→3)-2,5,6-tri-O-benzoyl-β-D-galactofuranoside C1

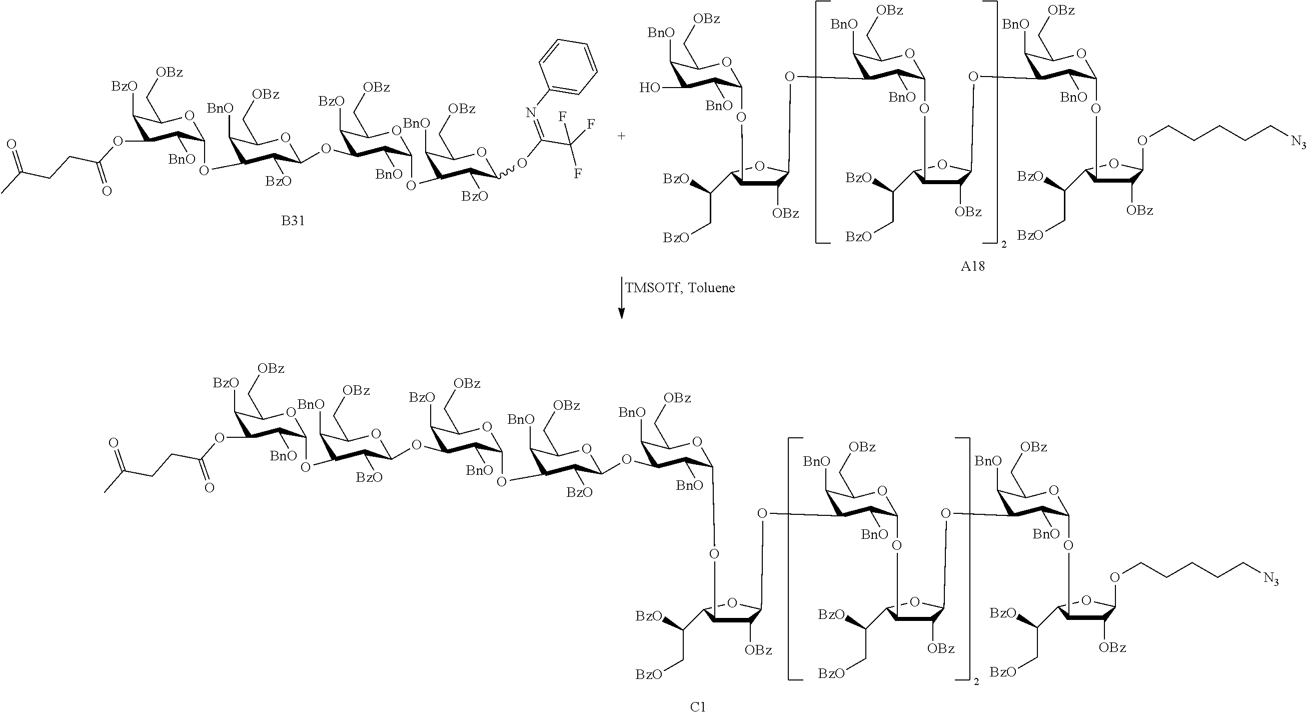

Both the Acceptor A18 (2 g, 0.525 mmol) and the donor B31 (1.23 g, 0.577 mmol) were taken in RBF and dried azeotropically using dry toluene in the vacuum. Mixture was taken in anhydrous toluene (15 mL) at rt, added 4 Å molecular sieves to it and stirred at for 30 min under N₂ atmosphere. Cooled the RM to −2 deg using Ice water bath and added TMSOTf (19 μL, 0.1 mmol) to the RM and stirred the RM at 5 deg for 20 mins. RM was then allowed to warm slowly to room temp over one hr. TLC analysis was carried out to monitor the completion of the reaction. RM was quenched with sat. NaHCO₃, stirred for 10 mins and extracted with EA. Combined organics were washed with water, brine, dried (Na₂SO₄), evaporated in vacuum to get crude product. Column purification on silica was done using EA/cyclohexane on Biotage®. Fractions containing product were evaporated and dried in vacuum to get desired product as fluffy white solid C1 (2.15 g, 71%). ¹H NMR (400 MHz, CDCl₃) δ 8.27-6.54 (m, 180H), 5.92-5.42 (m, 13H), 5.40-4.84 (m, 17H), 4.77 (d, J=3.8 Hz, 1H), 4.73-3.45 (m, 87H), 3.30 (dt, J=9.6, 6.4 Hz, 1H), 3.11 (t, J=6.9 Hz, 3H), 2.80-2.24 (m, 4H), 2.05 (s, 3H), 1.57-1.39 (m, 4H), 1.36-1.17 (m, 2H). MALDI-TOF Calcd for C₃₃₄H₃₀₆N₃O₈₇⁺ [M+H]⁺ 5749.9607, found 5749.992.

### Synthesis of Dodecasaccharide Acceptor C2

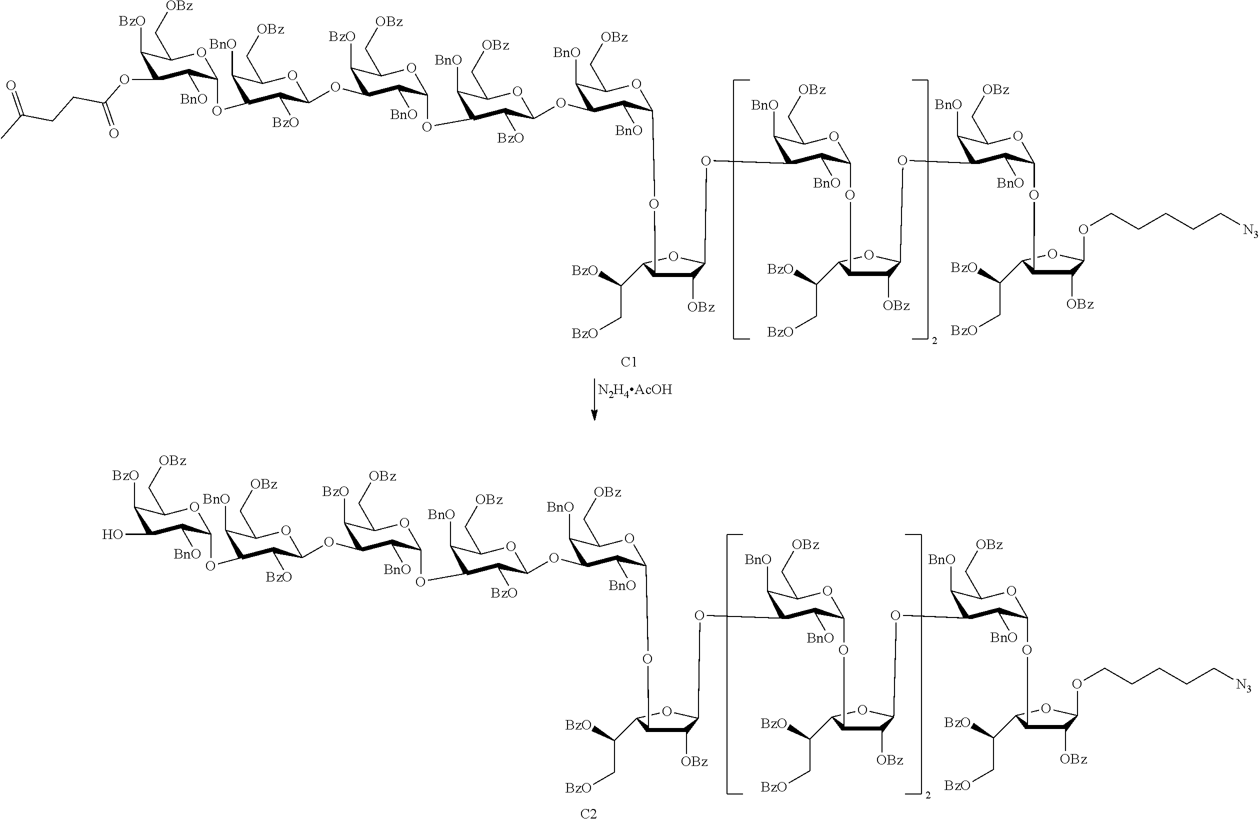

C1 Lev-substrate (2.1 g, 0.365 mmol) was taken in DCM-Pyridine (25 mL-0.5 mL) at rt, added hydrazine acetate (0.5 g, 5.48 mmol) to it and stirred at rt for 20 h. TLC analysis showed the completion of the reaction. RM was quenched with acetone (5 mL) and stirred for an hour before evaporated in vacuum to get crude. Purified by silica column on Biotage® using EA/Cy as eluents. Fractions containing product were collected and evaporated in vacuum to get desired product as fluffy white solid. The product fraction was taken for SEC column on LH-20 using 60% CHCl₃/Methanol as eluent to get product SEC fractions, evaporated in vacuum and dried to afford white fluffy solid C2 (1.9 g, 92%). MALDI-TOF Calcd for C₃₂₉H₃₀₀N₃O₈₅⁺ [M+H]⁺ 5651.9239, found 5651.881.

### Synthesis of 5-azido-pentyl 4,6-di-O-benzoyl-2-O-benzyl-3-O-levulinoyl-α-D-galactopyranosyl-(1→3)-2,6-di-O-benzoyl-4-O-benzyl-β-D-galactopyranosyl-(1-3)-4,6-di-O-benzoyl-2-O-benzyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1-+3)-2,5,6-tri-O-benzoyl-β-D-galactofuranosyl-(1-+3)-6-O-benzoyl-2,4-di-O-benzyl-α-D-galactopyranosyl-(1→3)-2,5,6-tri-O-benzoyl-β-D-galactofuranosyl-(1→3)]-6-O-benzoyl-2,4-di-O-benzyl-α-D-galactopyranosyl-(1→3)-2,5,6-tri-O-benzoyl-β-D-galactofuranoside C3

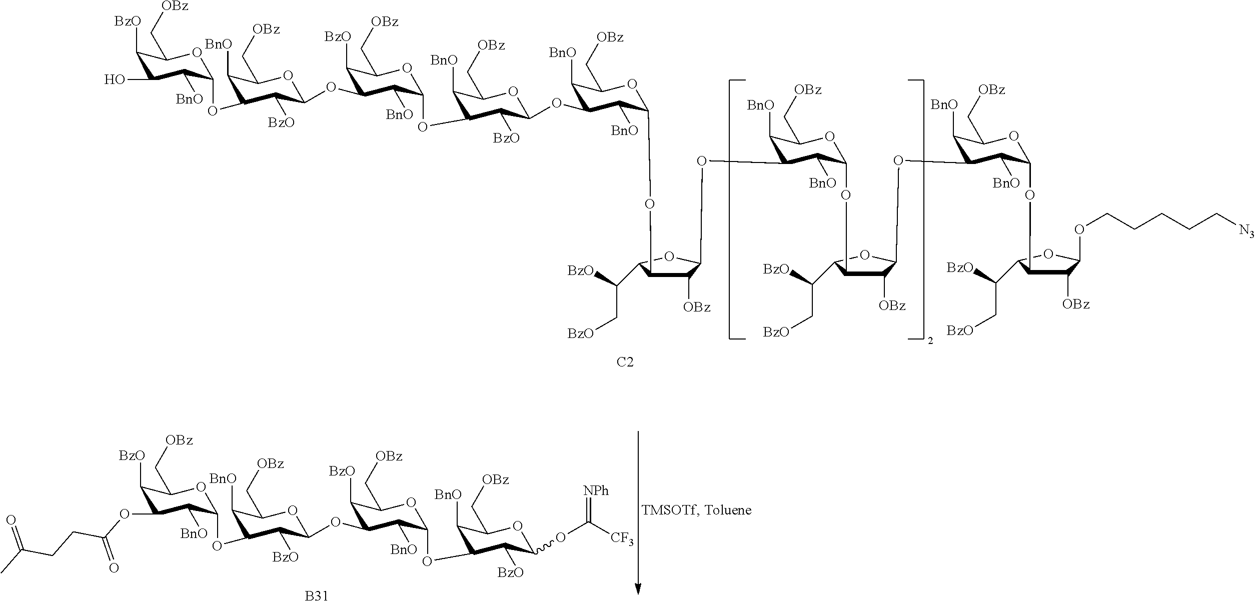

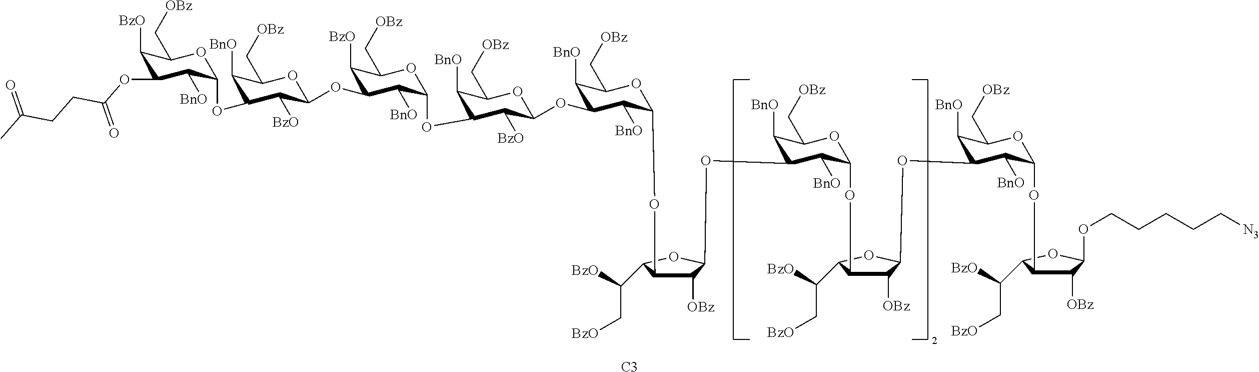

Both the Acceptor C2 (1.9 g, 0.336 mmol) and the donor B31 (0.83 g, 0.386 mmol) were taken in RBF and dried azeotropically using dry toluene in the vacuum. Mixture was taken in anhydrous toluene (20 mL) at rt, added 4 Å molecular sieves to it and stirred at for 30 min under N₂ atmosphere. Cooled the RM to −2 deg using ice water bath and added TMSOTf (12 μL, 0.067 mmol) to the RM and stirred the RM at 5 deg for 20 mins. RM was then allowed to warm slowly to room temp over one hr. TLC analysis was carried out to monitor the completion of the reaction. RM was quenched with sat. NaHCO₃, stirred for 10 mins and extracted with EA. Combined organics were washed with water, brine, dried (Na₂SO₄), evaporated in vacuum to get crude product. Column purification on silica was done using EA/cyclohexane on Biotage®. Fractions containing product were evaporated and dried in vacuum to get desired product as fluffy solid C3 (2.15 g, 84%). ¹H NMR (400 MHz, CDCl₃) δ 8.29-6.55 (m, 240H), 5.96-5.30 (m, 16H), 5.29-5.12 (m, 4H), 5.12-4.82 (m, 16H), 4.75 (d, J=3.8 Hz, 1H), 4.72-3.03 (m, 111H), 2.77-2.23 (m, 4H), 2.05 (s, 3H), 1.55-1.38 (m, 4H), 1.37-1.19 (m, 2H). MALDI-TOF Calcd for C₄₄₂H₄₀₂N₃O₁₁₅⁺ [M+H]⁺ 7590.5695, found 7590.489.

### Synthesis of Hexadecasaccharide Acceptor C4

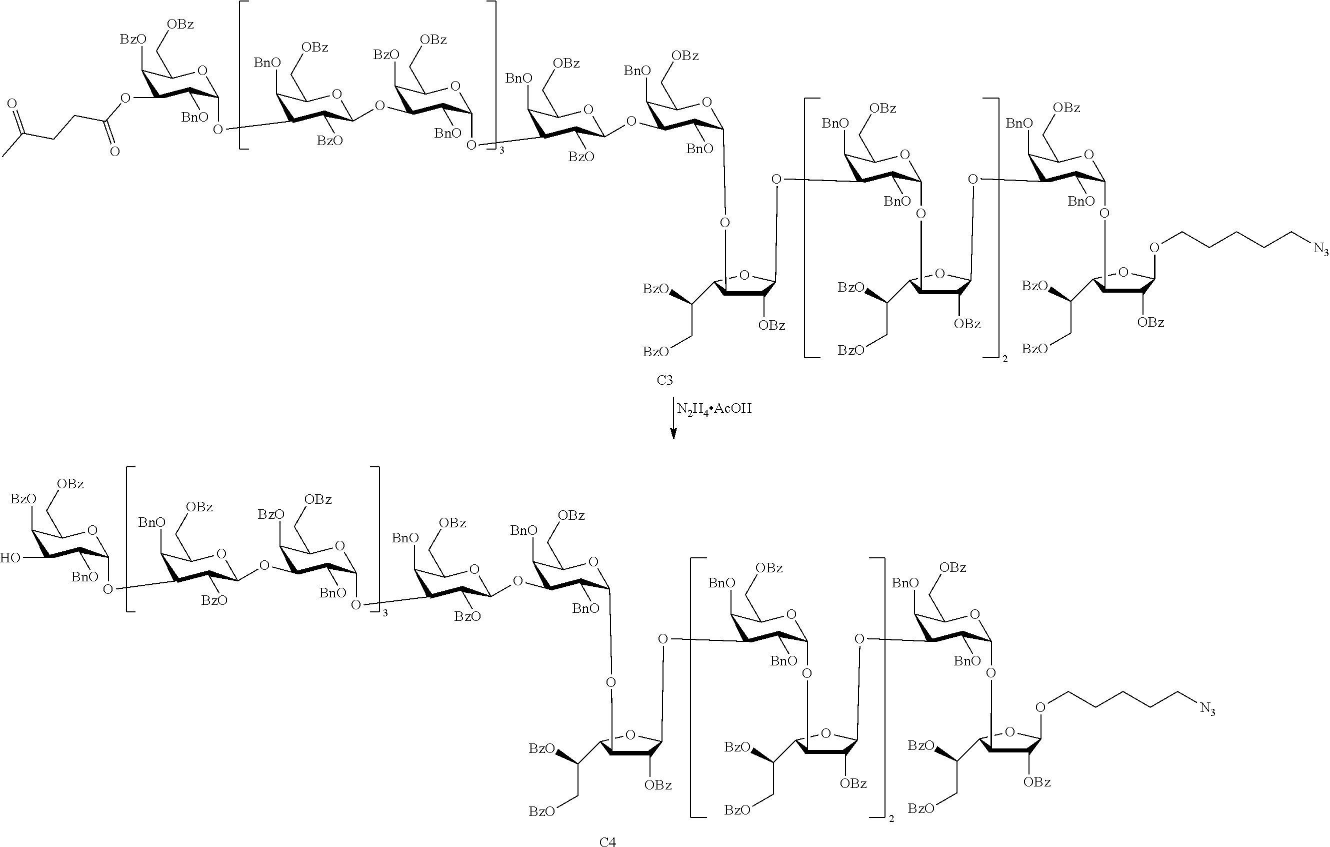

Lev-substrate C3 (2.1 g, 0.276 mmol) was taken in DCM-Pyridine (25 mL-0.5 mL) at rt, added hydrazine acetate (0.38 g, 4.15 mmol) to it and stirred at rt for 20 h. RM was quenched with acetone (5 mL) and stirred for an hour before evaporated in vacuum to get crude. Purified by silica column on Biotage® using EA/Cy as eluents. Fractions containing product were collected and evaporated in vacuum to get desired product as fluffy white solid C4 (1.91 g, 92%). MALDI-TOF Calcd for C₄₃₇H₃₉₆N₃O₁₁₃⁺ [M+H]⁺ 7492.5327, found 7492.520.

### Synthesis of 5-azido-pentyl 4,6-di-O-benzoyl-2-O-benzyl-3-O-levulinoyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1-3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-4,6-di-O-benzoyl-2-O-benzyl-α-D-galactopyranosyl-(1→3)-2,6-di-O-benzoyl-4-O-benzyl-β-D-galactopyranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1-+3)-6-O-benzoyl-2,4-di-O-benzyl-α-D-galactopyranosyl-(1-3)-2,5,6-tri-O-benzoyl-β-D-galactofuranosyl-(1→3)]-6-O-benzoyl-2,4-di-O-benzyl-α-D-galactopyranosyl-(1→3)-2,5,6-tri-O-benzoyl-β-D-galactofuranoside C5 using (4+16) approach

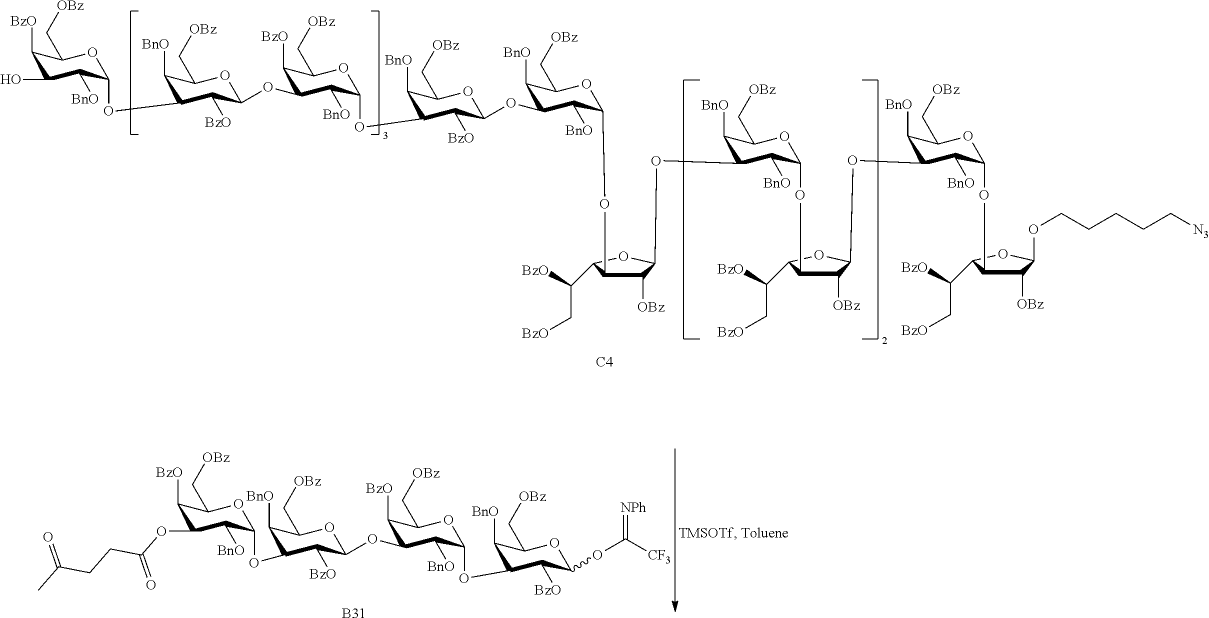

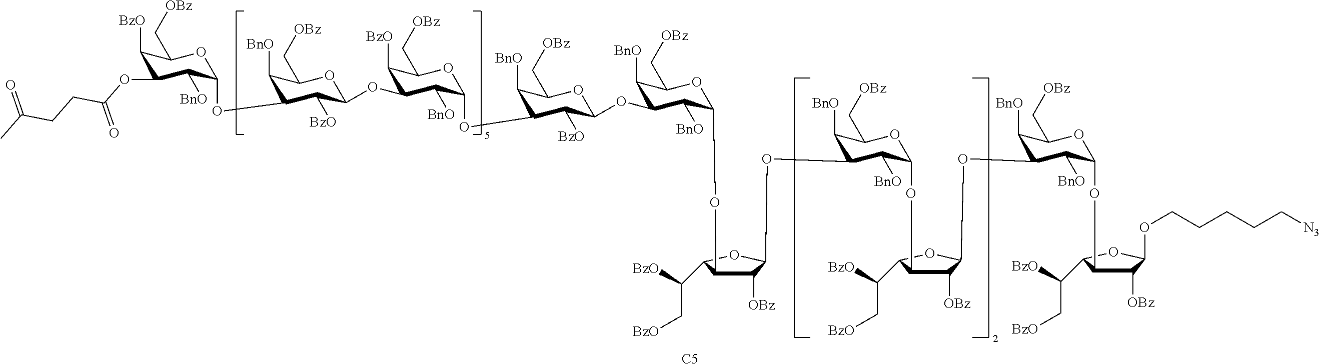

Both the Acceptor C4 (1.85 g, 0.245 mmol) and the donor B31 (0.6 g, 0.282 mmol) were taken in RBF and dried azeotropically using dry toluene in the vacuum. Mixture was taken in anhydrous toluene (20 mL) at rt, added 4 Å molecular sieves to it and stirred at for 30 min under N₂ atmosphere. Cooled the RM to −2 deg using Ice water bath and added TMSOTf (9 UL, 0.05 mmol) to the RM and stirred the RM at 5 deg for 20 mins. RM was then allowed to warm slowly to room temp over one hr. TLC analysis was carried out to monitor the completion of the reaction. RM was quenched with sat. NaHCO₃, stirred for 10 mins and extracted with EA. Combined organics were washed with water, brine, dried (Na₂SO₄), evaporated in vacuum to get crude product. Column purification on silica was done using EA/cyclohexane on Biotage®. Fractions containing product were evaporated and dried in vacuum to get desired product. Which was further purified by SEC column using LH-20 resin and 60% CHCl3 in MeOH as eluents. Fractions containing product were evaporated and dried in vacuum to get desired product C5 (1.35 g, 58%). MALDI-TOF Calcd for C₅₅₀H₄₉₇N₃NaO₁₄₃⁺ [M+Na]⁺ 9453.1603, found 9453.779.

### Synthesis of C5 Using (12+8) Approach

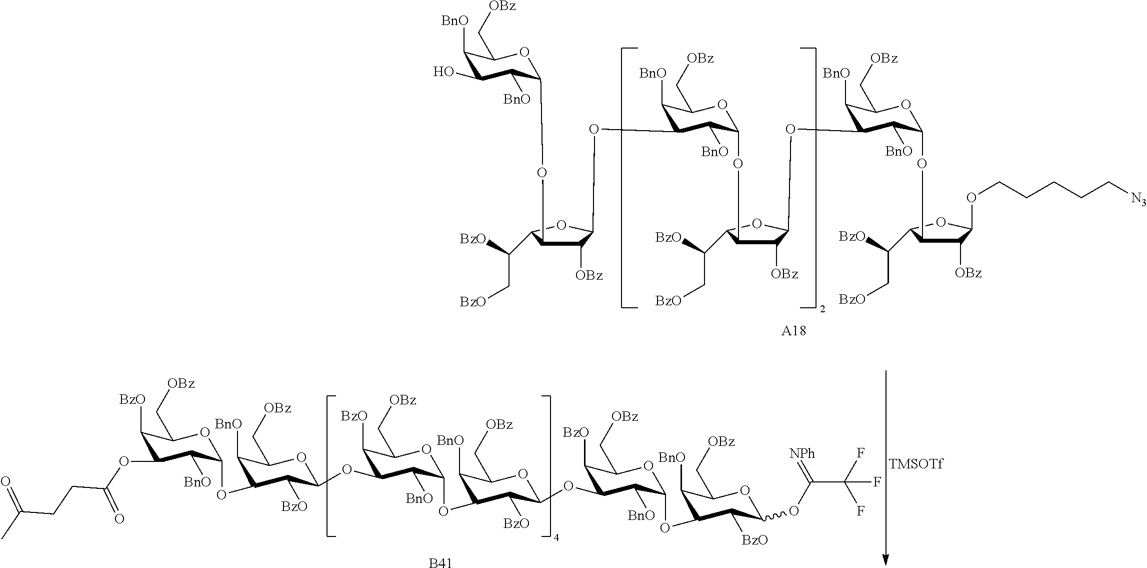

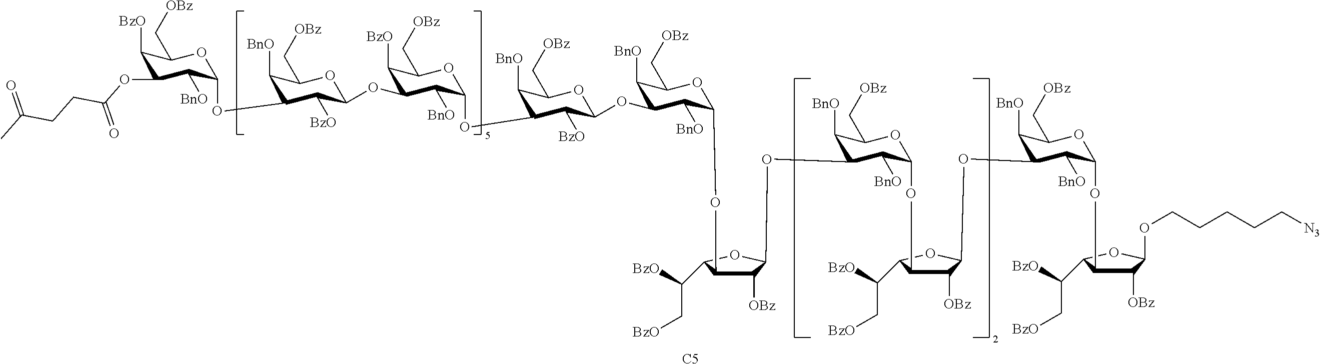

A18 (500 mg, 0.131 mmol) and B41 (991 mg, 0.170 mmol) were taken in DCM (25 mL) at rt, added 4 Å MS to it and stirred for 45 min. Cooled the RM to −10° C. using ice-acetone bath and added TMSOTf (5 μL, 0.028 μmol). The RM was stirred at −10° C. for 5 min and slowly warmed to 5° C. over 1 h. Reaction completion was monitored using TLC analysis (45% EtOAc/Cy). RM was quenched with NaHCO₃ solution (30 mL), stirred for 10 min, separated the layers. The aqueous layer was extracted with DCM (10 mL×2). Combined organic layer was washed with water (20 mL), brine solution (20 mL), dried over Na₂SO₄, filtered, and evaporated in vacuum. Crude material was purified by the silica column chromatography using EtOAc/Cy to get fractions containing product, on evaporation under vacuum yielded desired product C5 (880 mg, 71%). ¹H NMR (400 MHz, CDCl₃) δ 8.30-6.52 (m, 300H), 5.95-5.31 (m, 18H), 5.29-5.13 (m, 6H), 5.09-4.79 (m, 20H), 4.75 (d, J=3.7 Hz, 1H), 4.69-2.98 (m, 139H), 2.77-2.24 (m, 4H), 2.05 (s, 3H), 1.58-1.39 (m, 4H), 1.37-1.20 (m, 2H).

### Synthesis of Partially Protected Icosasaccharide C6

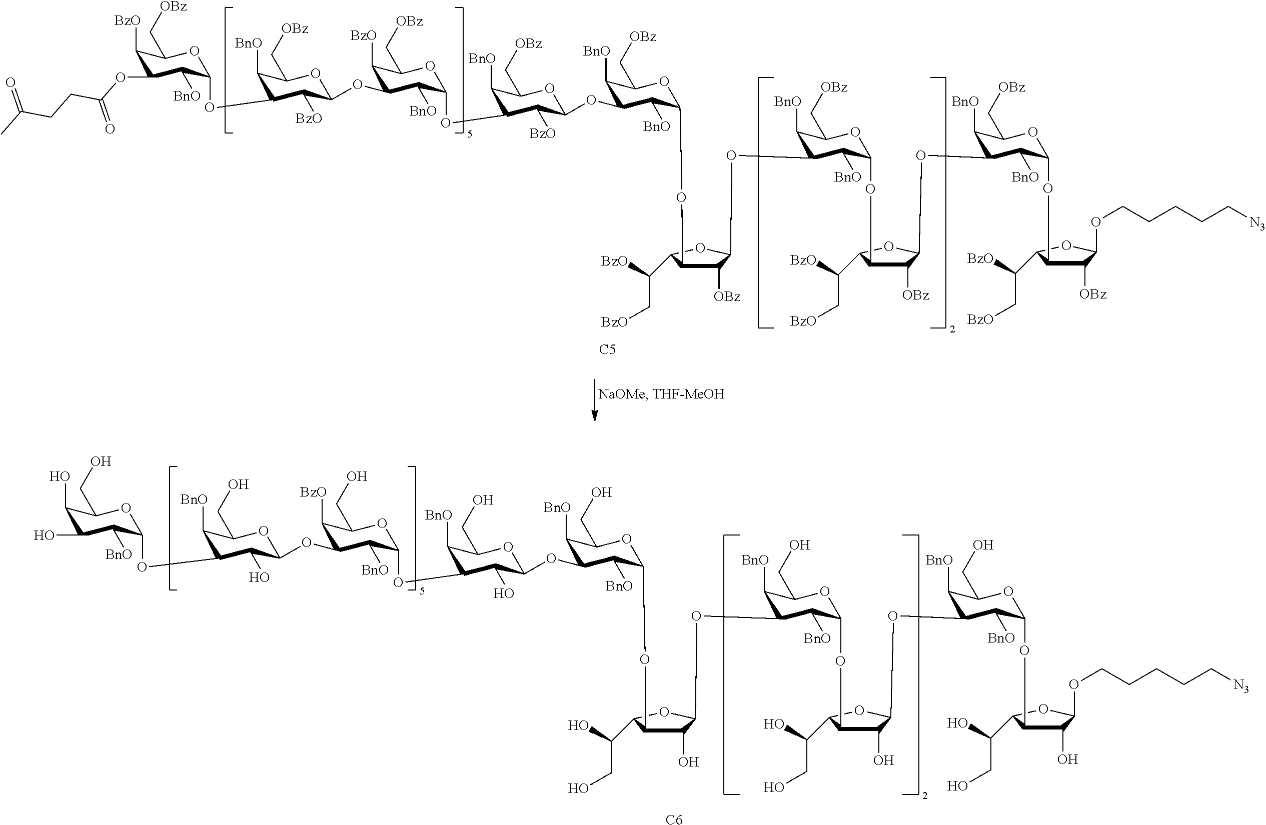

Substrate C5 (1.8 g, 0.191 mmol) was taken in 15 mL THF at rt, added excess 0.5 M NaOMe methanolic solution (28.6 mL, 14.31 mmol) to it and continued stirring at 55° C. for 20 h. Then added 0.5 ml of water to it and continued stirring for one more day. RM was cooled down to rt and evaporated to dryness. Water added to the residue and mixed very well. All colored solid dissolved except some off-white colored solid. So, filtered through syringe filters equipped with PTFE bed, washed the residue with warm water (10 mL×5). The solid was washed with very dilute acetic acid in water (5 drops of AcOH in 25 mL of water) and then washed with hot water (10 mL×3). The solid was dried in rotary evaporator to get pale yellow solid, analyzed by nmr and MALDI. The product was further purified on LH-20 SEC column using Methanol-CHCl₃ as eluents and collected product fractions, evaporated, and dried in vacuum to yield desired product as pale yellowish solid C6 (834 mg, 84%). MALDI-TOF Calcd for C₂₆₅H₃₃₂N₃O₁₀₁⁺ [M+H]⁺ 5172.0930, found 5172.072.

### Synthesis of 5-amino-pentyl α-D-galactopyranosyl-(1→3)-β-D-galactopyranosyl-(1-+3)-α-D-galactopyranosyl-(1→3)-β-D-galactopyranosyl-(1→3)-α-D-galactopyranosyl-(1-3)-β-D-galactopyranosyl-(1→3)-α-D-galactopyranosyl-(1→3)-β-D-galactopyranosyl-(1→3)-α-D-galactopyranosyl-(1→3)-β-D-galactopyranosyl-(1→3)-α-D-galactopyranosyl-(1→3)-β-D-galactopyranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranoside C7

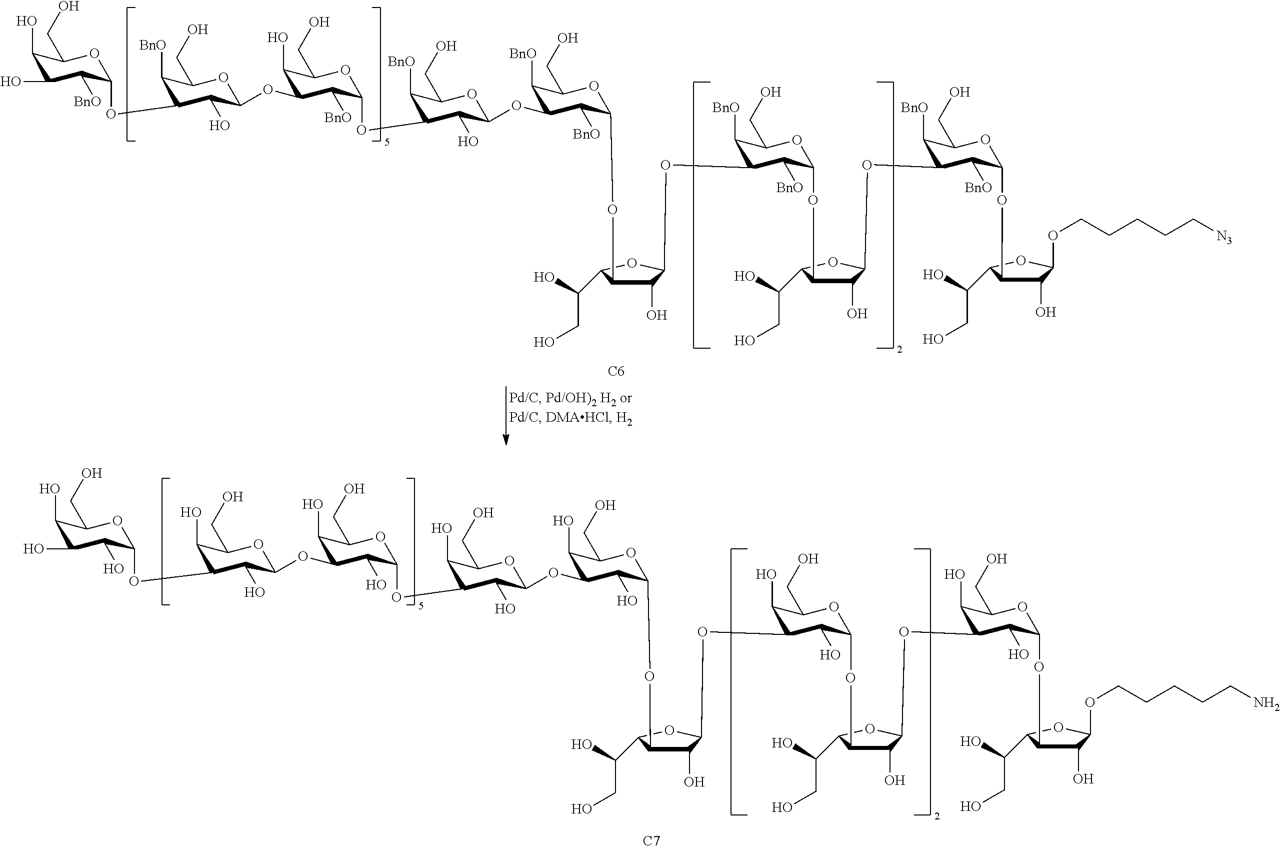

Substrate C6 (60 mg) was taken in mixture of IPA:EA:water (1.5:1.25:1) as a hazy mixture, added AcOH (25 μL), Pd/C (30 mg) and Pd(OH)₂ (30 mg) to it and hydrogenated under ˜11 bar H₂ atmosphere for 20 h. RM was filtered through the PTFE filter, washed with methanol and 50% methanol in water. The filtrate was concentrated under vacuum to get crude product which was purified using C18-sepak column with water-acetonitrile as eluents. All the fractions were frozen and lyophilized to dryness and analyzed by nmr and Maldi. Product fraction was further purified using SEC on G-25 resin using water as the eluent. Product fractions from SEC were collected, frozen, lyophilized to dryness to yield fluffy white solid as the desired product C7 (13.23 mg, 34%). Or

Substrate C6 (50 mg) was taken in mixture of IPA:EA:water:PBS (3:1:1:0.5) mL as a hazy mixture, added AcOH (10 μL), and stirred for 5 min. Pd/C (100 mg) was taken in IPA:water (1:0.5) mL solvent mixture, added dimethylamine hydrochloride (4 mg) to it and mixed well and kept at rt for 15 min. Transferred this Pd/C suspension to the vial containing substrate in solvent mixture and hydrogenated under ˜5 bar H₂ atmosphere for 20-24 h. RM was filtered through the PTFE filter, washed with methanol and water. The filtrate was concentrated under vacuum to get crude product which was purified using C18-sepak column with water-acetonitrile as eluents. All the fractions were frozen and lyophilized to dryness and analyzed by nmr and Maldi. Product fraction was further purified using SEC on G-25 resin using water as the eluent. Product fractions from SEC were collected, frozen, lyophilized to dryness to yield fluffy white solid as the desired product C7 (12.9 mg, 40%). ¹H NMR (400 MHz, D₂O) δ 5.18 (s, 3H), 5.16 (d, J=3.9 Hz, 5H), 5.12 (d, J=4.0 Hz, 1H), 5.06 (s, 4H), 5.01 (s, 1H), 4.70-4.56 (m, 6H), 4.39 (s, 3H), 4.32-3.53 (m, 119H), 2.98 (t, J=7.6 Hz, 2H), 1.72-1.59 (m, 4H), 1.48-1.37 (m, 2H). MALDI-TOF Calcd for C₁₂₅H₂₁₃NNaO₁₀₁⁺ [M+Na]⁺ 3367.1454, found 3367.167.

### Synthesis of Comparison Compound “Compar”

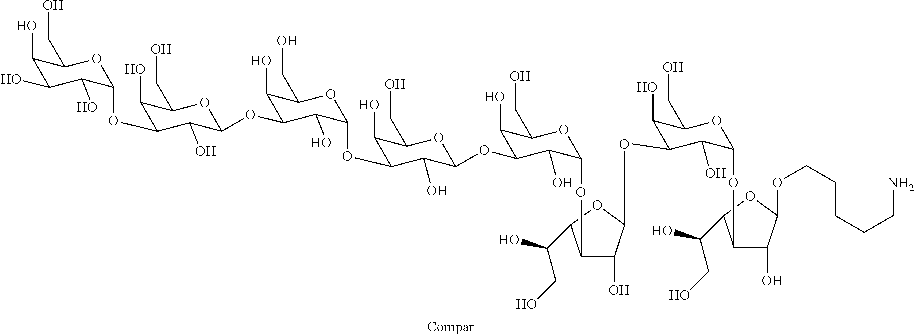 [SMILES: NCCCCCOC1OC([C@H](O)CO)C(OC2OC(CO)C(O)C(OC3OC([C@H](O)CO)C(OC4OC(CO)C(O)C(OC5OC(CO)C(O)C(OC6OC(CO)C(O)C(OC7OC(CO)C(O)C(OC8OC(CO)C(O)C(O)C8O)C7O)C6O)C5O)C4O)C3O)C2O)C1O]

The synthesis of the octasaccharide compound Compar is described in WO2019106201 on page 178 as compound 61*.

### Synthesis of the Conjugates

### Synthesis of the NHS Ester of Compound C7 (C7-Adipate-NHS)

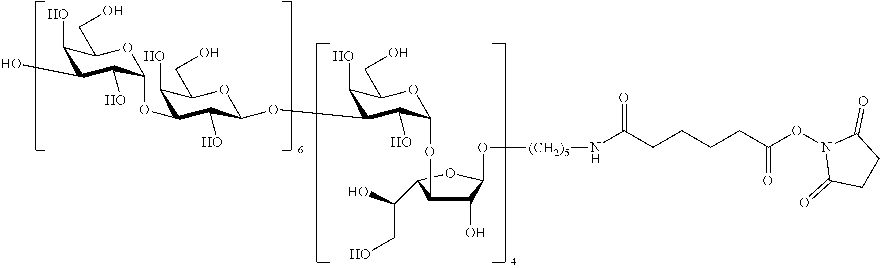

Compound C7 (8 mg, 2.391 μmol) was dissolved in DMSO-H₂O (400 μL-10 μL) at rt in a 15 mL falcon tube. Triethylamine (12 μL, 0.084 mmol) was added to it. The activated adipate-NHS ester bis(2,5-dioxopyrrolidin-1-yl) adipate (16.3 mg, 0.048 mmol) in DMSO (350 μL) was added and stirred for 2 h at rt. Compound C7-adipate-NHS was precipitated by adding EtOAc (9 mL) and centrifuged, washed the precipitate with EtOAc (5 mL×2), dried in vacuum to get white solid (8 mg, 94%) and taken for the next step.

### Synthesis of the C7 Conjugate with CRM₁₉₇ (C7-Adipate-CRM₁₉₇ or C7-CRM₁₉₇*)

Compound C7-adipate-NHS was dissolved in (8 mg, 2.24 μmol) in 0.1 M NaPi buffer (pH 7.0, 100 μL) in a 15 mL falcon tube. Freshly washed CRM₁₉₇ (obtained from EirGenix, Inc., Taiwan, expression system E. coli) (3 mg, 0.051 μmol) in 0.1 M NaPi buffer (pH 7.0, 200 μL) in a vial was added to it dropwise. The vial was rinsed with 0.1 M NaPi buffer (pH 7.0, 100 μL) and transferred to the reaction mixture in falcon tube and stirred at rt for 20 h. Obtained C7-adipate-CRM₁₉₇ solution was transferred to the Amicon Ultra vial (10 kDa, MWCO), centrifuged for 5 minutes at 2-8° C. temperature. Added 300 μL of 0.1 M NaPi to the reaction falcon tube, rinsed and transferred to the filter and centrifuged again. Additional washings were done using 1×PBS solution for five more times. After the final wash the conjugate was sterile-filtered and stored in PBS (1.5 mL) (pH 7.4) at 2-8° C. The loading found was 13.47 using MALDI-TOF MS. The conjugate was analysed using, SDS-PAGE, BCA protein determination, endotoxin content and SEC-HPLC.

### Synthesis of the Conjugate with BSA (C7-Adipate-BSA or C7-BSA*)

Compound C7-adipate-NHS was dissolved in (5.96 mg, 1.67 μmol) in 0.1 M NaPi buffer (pH 7.0, 200 μL) in a 15 mL falcon tube. Freshly washed BSA (obtained from Sigma Aldrich, heat shock fraction, pH 7, ≥98%; product no. A7906) (3 mg, 0.045 μmol) in 0.1 M NaPi buffer (pH 7.0, 200 μL) in a vial was added to it dropwise. The vial was rinsed with 0.1 M NaPi buffer (pH 7.0, 50 μL) and transferred to the reaction mixture in falcon tube and stirred at rt for 20 h. Obtained C7-adipate-BSA solution was transferred to the Amicon Ultra vial (10 kDa, MWCO), centrifuged for 5 minutes at 2-8° C. temperature. Added 300 μL of 0.1 M NaPi to the reaction falcon tube, rinsed and transferred to the filter and centrifuged again. Additional washings were done using 1×PBS solution for five more times. After the final wash the conjugate was sterile-filtered and stored in PBS (1.35 mL) (PH 7.4) at 2-8° C. The loading found was 11.86 using MALDI-TOF MS. The conjugate was analysed using SDS-PAGE and SEC-HPLC.

### Synthesis of the PNP Ester of Compound Compar (Compar-Adipate-PNP)

The synthesis of Compar-adipate-PNP is described in WO2019106201 on page 253 as PNP-ester synthesis of compound 61*.

### Synthesis of the Compar Conjugate with CRM₁₉₇ and BSA (Compar-Adipate-CRM₁₉₇ or Compar-CRM₁₉₇+, Compar-Adipate-BSA or Compar-BSA*)

The synthesis of Compar-CRM₁₉₇* is described in WO2019106201 on page 253-254 as conjugation of compound 61* to either CRM₁₉₇ or BSA.

### II. Biology

### Materials

ELISA plates (high-binding, EIA/RIA Plate, 96 well, flat bottom with low evaporation lid, company: Costar® 3361).
Detection antibody: Goat anti rabbit IgG peroxidase conjugate (Sigma, #A4914) and Goat anti-Mouse IgG (H+L) peroxidase conjugate (Dianova Code: 115-035-068).
Blocking solution: Commercial blocking reagent (Roche, cat. no. 11112589001)
Antibody diluent: PBS+1% BSA (w/v).
Wash Buffer: PBS+0.1% Tween 20 (PBS-T)
Developing solution: 1 Step™ Ultra TMB-ELISA developer. (ThermoScientific, Cat #: 34028)
Stop solution—2M sulphuric acid (H₂SO₄).
Plate reader: Anthos HT 2 or FLUOstar Omega (BMG LABTECH).
Software: GraphPad Prism 7 for data plotting and analysis.
Alum: Aluminium Hydroxide Gel Adjuvant (Alhydrogel® 2%), Brenntag, Batch #:5447 Exp Dt: February 2020.
QuantiPro™ BCA Assay Kit (SIGMA) Product: QPBCA-1KT; Lot #: SLBR7451V; Pcode: 1002296464.
Mini-PROTEAN® TGX™ Gels-10%, 10 well (30 μL/well) Control Nr: 64175708,
GelCode™ Blue Safe Protein Stain; ThermoScientific; Ref: 1860957; Lot #: TA260266

### Methods

### Bacterial Strains and LPS.

Klebsiella pneumoniae (KPC) strains differing in their LPS (O-antigen) with/without the capsule were used to isolate and purify the corresponding LPS. The purified LPS were used as coating antigen in Enzyme Linked Immunosorbent Assay (ELISA). LPS was isolated using a commercial LPS extraction kit (JH Science) according to the manufacturer's protocol.

TABLE 1
Klebsiella pneumoniae strains used for LPS isolation.

| # | LPS/O-antigen |
|---|---|
| 1 Friedländer or PCM12 | O1 |
| 2 NCTC 9148 | O2a |
| 3 PCM27 | Galactan-III |

Formulation of vaccine candidates for immunization. All formulations were prepared under sterile conditions. Drug substance (DS) and buffer (PBS or TBS) were mixed in the appropriate pre-calculated dilution factor (see below) for the required glycan dose leaving out the required volume of aluminum hydroxide adjuvant (0.25 mg/mL). The DS-buffer mixture was gently mixed and aluminum hydroxide adjuvant (“aluminum”) stock was added for a final aluminum concentration of 0.250 mg/ml of aluminum. The mixture was immediately mixed by gentle pipetting and then mixed on a horizontal shaker at 250 rpm for 2 h at RT. Aliquots were stored in type 1 glass vials at 4° C. until further use.

The vaccines described above are prepared to contain the intended glycan dose (e.g., 2 μg glycan per injection) as follows. The average loading factor of the glycan antigen (in moles of antigen per mol carrier protein) is determined via MALDI-TOF MS by subtracting the determined molecular weight (m/z=1) of CRM₁₉₇ from the determined molecular weight of the DS (m/z=1), then this mass difference is divided by the theoretical molecular weight of the glycan antigen including the linker (here: alkyl) and spacer (here: adipoyl) moieties. The resulting loading factor is multiplied by the theoretical molecular weight of the glycan antigen excluding the linker and spacer moieties, providing the total mass of glycan attached on average per DS molecule. This total mass of glycan is divided by the determined molecular weight of the CRM₁₉₇ protein to yield the glycan-to-protein mass ratio of the DS. This ratio is multiplied by the determined protein concentration of the DS, as determined by the BCA Assay Kit (Sigma) according to the manufacturer's protocol, to yield the glycan concentration of the DS. To obtain the dilution factor necessary to dilute the DS to obtain the intended glycan dose per immunization, the glycan concentration of the DS is divided by the required glycan concentration (e.g., 20 μg/mL glycan concentration for a 2 μg glycan dose for mice with an injection volume of 100 μL). The DS is then diluted with this dilution factor relative to the final volume of the vaccine preparation.

Immunizations: Female Zika rabbits were immunized via the intramuscular (i.m.) route with an injection volume of 500 μL per dose. Female mice were immunized via the subcutaneous (s.c.) route with an injection volume of 100 μL per dose. Animals were kept under specific pathogen-free conditions and were provided with water and food ad libitum.

ELISA: Coating of plates with antigen: Antigen-BSA glycoconjugates (C7-BSA* and Compar-BSA*) and isolated LPS were used for coating. LPS was dissolved in isopropanol to a concentration of 10 μg/mL and 100 μL was used for coating so that each well was coated with 1 μg of LPS. LPS solutions were subjected to overnight evaporation at RT inside the biosafety cabinet. The antigen-BSA glycoconjugates were diluted to 2 μg/mL in PBS and 50-100 μL (0.1-0.2 μg) were coated per well and incubated overnight at 4° C. Blocking: The plates were blocked using 100 μL of commercial blocking solution and incubated for 1 h at RT. After blocking, the plates were washed 3× with PBS with 0.1% (v/v) Tween-20 (PBS-T). Incubation with diluted sera: Pooled or individual sera from different timepoints were diluted to their respective dilutions using 1% BSA (w/v) in PBS. 50-100 μL of the diluted sera were added in duplicates to the ELISA wells and incubated for 1 h at RT. 100 μL/well of 1% BSA (w/v) in PBS served as blank. After incubation with sera, the plates were washed 3× with PBS-T. Incubation with detection antibody: Anti-mouse or anti-rabbit IgG HRP conjugate was diluted 1:10,000 in 1% BSA (w/v) in PBS and 100 μL/well were added and incubated for 30 minutes at RT. After the incubation with detection antibody, the plates were washed 3× with PBS-T. Substrate addition: To each well, 100 μL of TMB substrate were added and incubated for approx. 15 min. The reaction was stopped by adding 50 μL/well of 2M H₂SO₄. Absorption was measured at 450 nm using a plate reader. The absorption values were analyzed with the GraphPad Prism software.

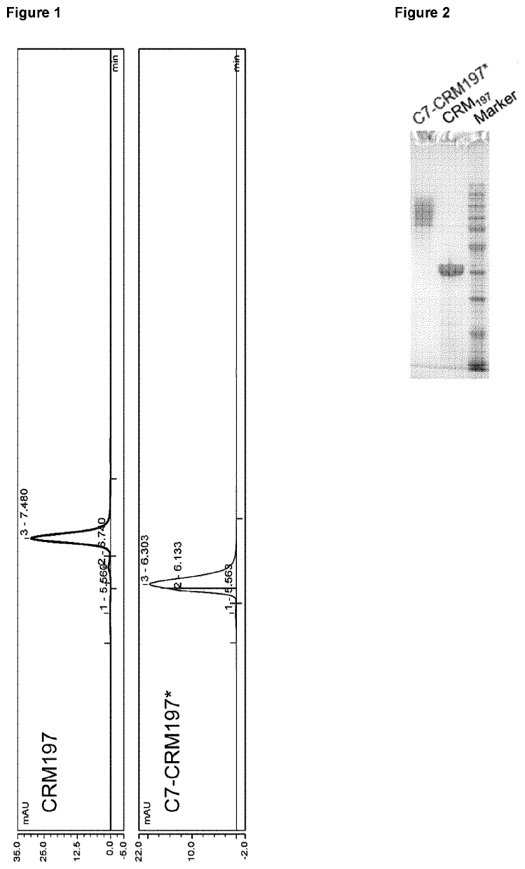

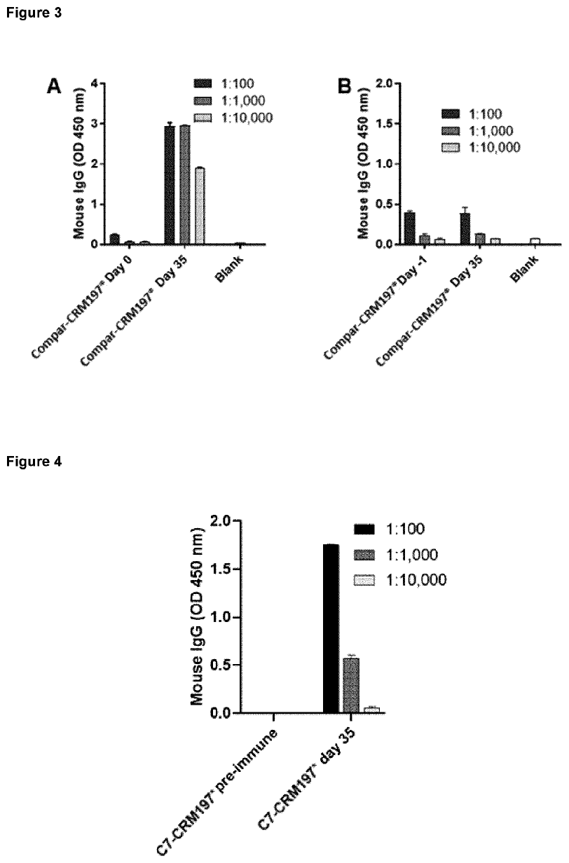

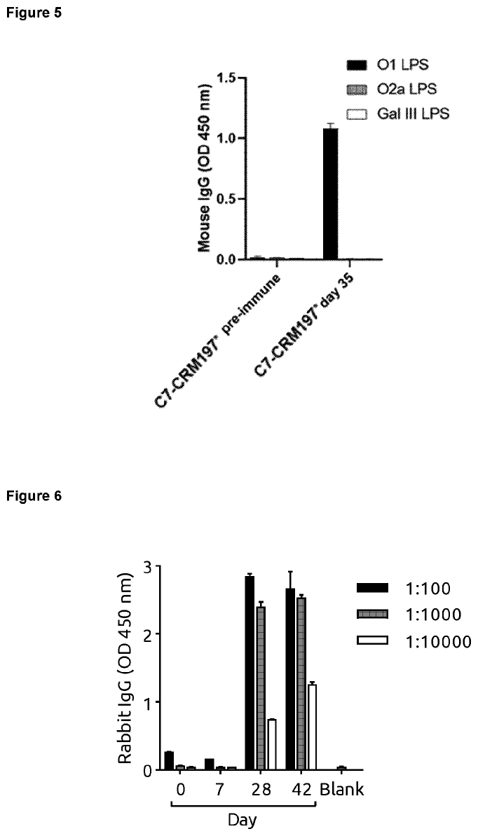

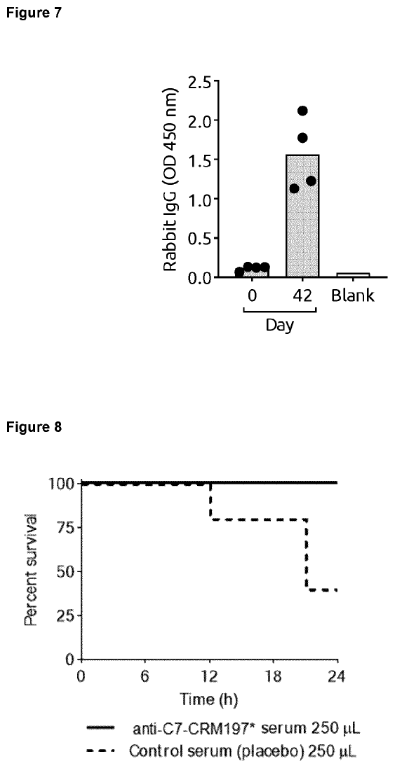

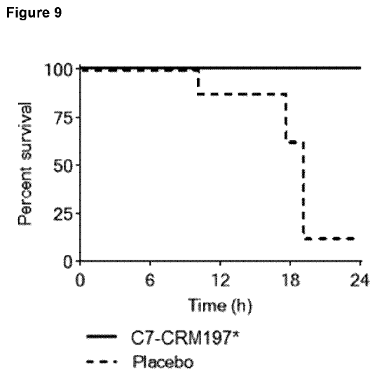

Results: The compound C7 was successfully conjugated to the carrier protein CRM₁₉₇, as shown by HPLC-SEC (FIG. 1) and SDS-PAGE (FIG. 2). A comparison conjugate compound (Compar-CRM₁₉₇* bearing octasaccharide antigens) was immunogenic as CRM₁₉₇-conjugate in mice (FIG. 3A) but the induced IgGs did not bind to isolated 01 LPS (FIG. 3B). C7-CRM₁₉₇* was also immunogenic in mice (FIG. 4) but in contrast to the comparison conjugate Compar-CRM₁₉₇* the induced IgGs recognized the natural antigen, isolated LPS of 01 (FIG. 5). C7-CRM₁₉₇* was also immunogenic in rabbits (FIG. 6) and the IgGs recognized the natural LPS antigen (FIG. 7). The C7-CRM197* induced antibodies were protective, as shown in a challenge experiment in which rabbit antisera were transferred into mice (passive immunization) followed by lethal infection with an O1-expressing K. pneumoniae strain (PCM12) (FIG. 8). Similarly, mice actively vaccinated with C7-CRM197* were significantly protected from O1 K. pneumoniae infection (PCM12) compared to placebo-immunized mice (FIG. 9).

SEQ ID No. 1:
amino acid sequence of CRM₁₉₇:
GADDVVDSSK SFVMENFSSY HGTKPGYVDS 30
IQKGIQKPKS GTQGNYDDDW KEFYSTDNKY 60
DAAGYSVDNE NPLSGKAGGV VKVTYPGLTK 90
VLALKVDNAE TIKKELGLSL TEPLMEQVGT 120
EEFIKRFGDG ASRVVLSLPF AEGSSSVEYI 150
NNWEQAKALS VELEINFETR GKRGQDAMYE 180
YMAQACAGNR VRRSVGSSLS CINLDWDVIR 210
DKTKTKIESL KEHGPIKNKM SESPNKTVSE 240
EKAKQYLEEF HQTALEHPEL SELKTVTGTN 270
PVFAGANYAA WAVNVAQVID SETADNLEKT 300
TAALSILPGI GSVMGIADGA VHHNTEEIVA 330
QSIALSSLMV AQAIPLVGEL VDIGFAAYNF 360
VESIINLFQV VHNSYNRPAY SPGHKTQPFL 390
HDGYAVSWNT VEDSIIRTGF QGESGHDIKI 420
TAENTPLPIA GVLLPTIPGK LDVNKSKTHI 450
SVNGRKIRMR CRAIDGDVTF CRPKSPVYVG 480
NGVHANLHVA FHRSSSEKIH SNEISSDSIG 510
VLGYQKTVDH TKVNSKLSLF FEIKS 535

1. An oligosaccharide-carrier protein conjugate of formula (I) 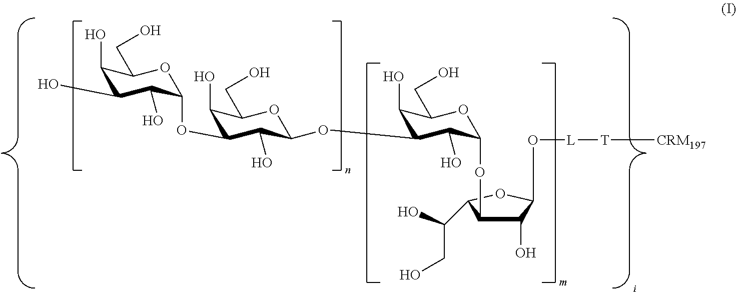
wherein
m is 4, 5 or 6;
n is 5, 6 or 7;
i is from 1 to 28; and
-L-T-represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, wherein the atoms of the backbone are selected from the group consisting of carbon, nitrogen, oxygen and sulphur;
or a pharmaceutically acceptable salt thereof.

2. The oligosaccharide-carrier protein conjugate according to claim 1, or a pharmaceutically acceptable salt thereof, wherein m is 4, and n is 6.

3. The oligosaccharide-carrier protein conjugate according to claim 1, or a pharmaceutically acceptable salt thereof, wherein the bridge does not contain an aromatic or heteroaromatic ring.

4. The oligosaccharide-carrier protein conjugate according claim 1, or a pharmaceutically acceptable salt thereof,
wherein
-L-T-represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, bearing at most one double bond,
wherein the atoms of the backbone are selected from the group consisting of carbon, nitrogen, oxygen and sulphur, and
wherein the backbone may be substituted with one or more substituents independently selected from oxo, (C₁₋₄)alkyl, fluoro, and (C₁₋₂)alkoxy, and
wherein a part of the backbone optionally may be part of a 4-, 5- or 6-membered ring selected from:

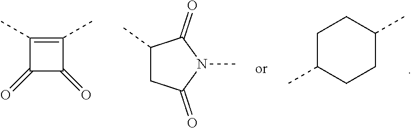

5. The oligosaccharide-carrier protein conjugate according to claim 1, or a pharmaceutically acceptable salt thereof, wherein
-L-T-represents a linker L and a spacer T which together form a bridge which consists of a backbone which is a saturated chain counting from 5 to 25 atoms selected from the group consisting of carbon, nitrogen, oxygen and sulphur, which chain may be unsubstituted or substituted with one or more substituents independently selected from oxo, (C₁₋₄)alkyl, fluoro and (C₁₋₂)alkoxy.

6. The oligosaccharide-carrier protein conjugate according to claim 1, or a pharmaceutically acceptable salt thereof, wherein the backbone of the bridge has a length of 8 to 20 atoms covalently linked together that forms the shortest distance between the oxygen at C1 of the reducing end of the oligosaccharide and the nitrogen of the amino group of a lysine residue at the carrier protein CRM₁₉₇.

7. The oligosaccharide-carrier protein conjugate according to claim 1, or a pharmaceutically acceptable salt thereof, wherein
L represents
*—(C₂₋₁₀)alkylene-NH—;
*—(CH₂CH₂O)ₐ—CH₂CH₂NH—, wherein a is 1, 2 or 3;
*—CH₂CH₂S—CH₂CH₂NH—;
*—(C₂₋₁₀)fluoroalkylene-NH—;
*—(CH₂)_{c}NHC(O)(CH₂)_{d}—NH—, wherein c and d are independently from each other from 2 to 6;
*—(CH₂)ₑNHC(O)NH(CH₂)ₕ—NH—, wherein e and h are independently from each other from 2 to 6;
*—(C₁₋₁₀)alkylene-C(O)—NH—(C₂₋₁₀)alkylene-NH—; or
*—(C₂₋₁₀)alkylene-O—NH—;
T represents
—C(O)—(C₀₋₁₀)alkylene-C(O)—;
—C(O)—CH₂CH₂—(OCH₂CH₂)ᵣ—C(O)—, wherein r is from 1 to 5;
—C(O)—CH₂(CH₂)_{f}—(SCH₂(CH₂)_{f′})_{f″}—C(O)—, wherein f is 0 or 1, f′ is 0 or 1, and f″ is 1, 2, or 3; 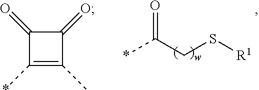
wherein w is 1 or 2; or 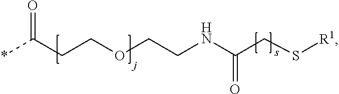
wherein j is from 1 to 4, and s is 1 or 2; or
L-T represents
*—(C₂₋₁₀)alkylene-S—R¹; and
R¹ represents 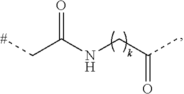
wherein k is 2 or 3; 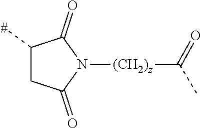
wherein z is 2 or 3; or

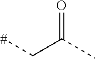

8. The oligosaccharide-carrier protein conjugate according to claim 7, or a pharmaceutically acceptable salt thereof, wherein
L represents
*—(CH₂)ₗ—NH—; wherein l is from 2 to 10;
*—(CH₂CH₂O)ₐ—CH₂CH₂NH—, wherein a is 1, 2 or 3;
*—CH₂CH₂S—CH₂CH₂NH—;
*—(C₂₋₁₀)fluoroalkylene-NH— with fluoroalkylene being a saturated straight chain;
*—(CH₂)_{c}NHC(O)(CH₂)_{d}—NH—, wherein c and d are independently from each other from 2 to 6;
*—(CH₂)ₑNHC(O)NH(CH₂)ₕ—NH—, wherein e and h are independently from each other from 2 to 6;
*—(CH₂)ᵤ—C(O)—NH—(CH₂)_{u′}—NH—; wherein u is from 1 to 10 and u′ is from 2 to 10; or
*—(CH₂)_{g}—O—NH—, wherein g is from 2 to 10; or
L-T represents
*—(CH₂)_{q}S—R¹, wherein q is from 2 to 10; and
T represents
—C(O)—(CH₂)ₚ—C(O)—, wherein p is from 0 to 10;
—C(O)—CH₂CH₂—(OCH₂CH₂)ᵣ—C(O)—, wherein r is from 1 to 5;
—C(O)—CH₂ (CH₂)_{f}—(SCH₂(CH₂)_{f′})_{f″}—C(O)—, wherein f is 0 or 1, f′ is 0 or 1, and f″ is
1, 2, or 3; 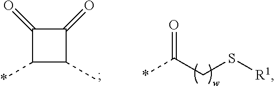
wherein w is 1 or 2; or 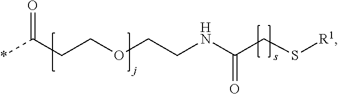
wherein J is from 1 to 4, and s is 1 or 2; and
R¹ represents 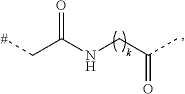
wherein k is 2 or 3; 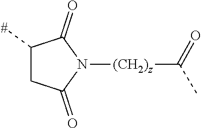
wherein z is 2 or 3; or

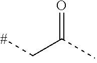

9. The oligosaccharide-carrier protein conjugate according to claim 7, or a pharmaceutically acceptable salt thereof, wherein
L represents
—(CH₂)ₗ—NH—; wherein l is from 2 to 10;
*—(CH₂CH₂O)ₐ—CH₂CH₂NH—, wherein a is 1, 2 or 3; or
*—(CH₂)_{g}—O—NH—, wherein g is from 2 to 10; and
T represents
—C(O)—(CH₂)ₚ—C(O)—, wherein p is from 0 to 10;
C(O)—CH₂CH₂—(OCH₂CH₂)ᵣ—C(O)—, wherein r is from 1 to 5;
—C(O)—CH₂(CH₂)_{f}—(SCH₂(CH₂)_{f′})_{f″}—C(O)—, wherein f is 0 or 1, f′ is 0 or 1, and f″ is 1, 2, or 3; or

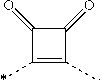

10. The oligosaccharide-carrier protein conjugate according to claim 7, or a pharmaceutically acceptable salt thereof, wherein
L is *—(CH₂)₅—NH—; and
T is —C(O)—(CH₂)₄—C(O)—.

11. The oligosaccharide-carrier protein conjugate according to claim 1, or a pharmaceutically acceptable salt thereof,
wherein i is from 6 to 15.

12. The oligosaccharide-carrier protein conjugate according to claim 1, or a pharmaceutically acceptable salt thereof,
wherein the oligosaccharide-carrier protein conjugate has the structure of formula (Ib): 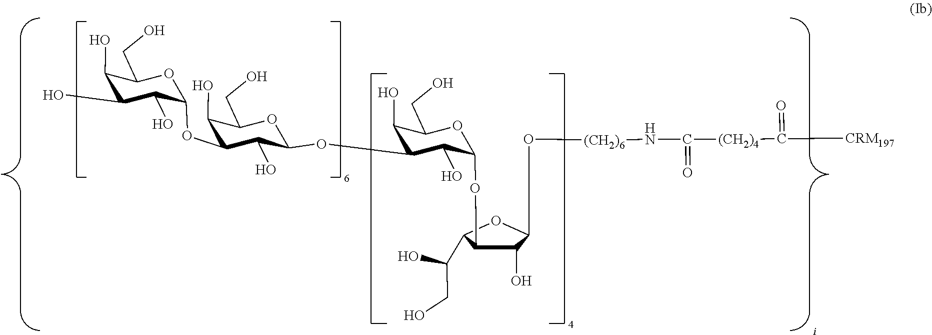
wherein i is from 1 to 28.

13. The oligosaccharide-carrier protein conjugate according to claim 12, or a pharmaceutically acceptable salt thereof, wherein i is from 6 to 15.

14. A pharmaceutical composition comprising, as active principle, an oligosaccharide-carrier protein conjugate according to claim 1, or a pharmaceutically acceptable salt thereof, and at least one therapeutically inert excipient.

15. The pharmaceutical composition according to claim 14, further comprising an adjuvant.

16. (canceled)

17. A method for prevention and/or treatment of a K. pneumoniae infection in a human and/or animal, wherein the method comprises administering an oligosaccharide-carrier protein conjugate according to claim 1, or a pharmaceutically acceptable salt thereof, to the human and/or animal.

18. The method according to claim 17, wherein the K. pneumoniae infection is selected from the group consisting of a K. pneumoniae infection in an individual of 50 years or older; a hospital acquired K. pneumoniae infection; a community-acquired K. pneumoniae infection; and pneumonia, bronchitis, meningitis, urinary tract infection, intra-abdominal infection, wound infection, infection of blood, osteomyelitis, bacteremia, septicemia, liver abscess, and inflammatory bowel disease (IBD), all caused by K. pneumoniae infection.

19. The method according to claim 17, wherein K. pneumoniae is selected from O-serotypes comprising O1.

20. A multivalent vaccine comprising the oligosaccharide-carrier protein conjugate according to claim 1, or a pharmaceutically acceptable salt thereof.

21. A compound having the formula (II) 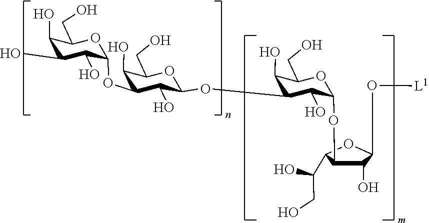
wherein
m is 4, 5 or 6, preferably 4 or 5, more preferably 4;
n is 5, 6 or 7, preferably 6 or 7, more preferably 6;
L¹ represents
*—(C₂₋₁₀)alkylene-NH₂, preferably *—(CH₂)ₗ—NH₂; wherein l is from 2 to 10, more preferably l is 5;
*—(CH₂CH₂O)ₐ—CH₂CH₂NH₂, wherein a is 1, 2 or 3;
*—CH₂CH₂S—CH₂CH₂NH₂;
*—(C₂₋₁₀)fluoroalkylene-NH₂;
*—(CH₂)_{c}NHC(O)(CH₂)_{d}—NH₂, wherein c and d are independently from each other from 2 to 6;
*—(CH₂)ₑNHC(O)NH(CH₂)ₕ—NH₂, wherein e and h are independently from each other from 2 to 6;
*—(C₁₋₁₀)alkylene-C(O)—NH—(C₂₋₁₀)alkylene-NH₂;
*—(C₂₋₁₀)alkylene-O—NH₂; or
*—(C₂₋₁₀)alkylene-SH;
or a pharmaceutically acceptable salt thereof.

22. A compound having the formula (III) 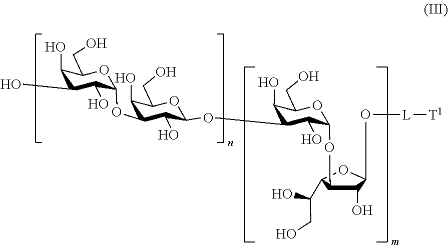
wherein
m is 4, 5 or 6, preferably m is 4 or 5, most preferably 4;
n is 5, 6 or 7, preferably n is 6 or 7, most preferably 6;
L represents
*—(C₂₋₁₀)alkylene-NH—, preferably *—(CH₂)ₗ—NH— wherein l is from 2 to 10, more preferably 5;
*—(CH₂CH₂O)ₐ—CH₂CH₂NH—, wherein a is 1, 2 or 3;
*—CH₂CH₂S—CH₂CH₂NH—;
*—(C₂₋₁₀)fluoroalkylene-NH—;
*—(CH₂)_{c}NHC(O)(CH₂)_{d}—NH—, wherein c and d are independently from each other from 2 to 6;
*—(CH₂)ₑNHC(O)NH(CH₂)ₕ—NH—, wherein e and h are independently from each other from 2 to 6;
*—(C₁₋₁₀)alkylene-C(O)—NH—(C₂₋₁₀)alkylene-NH—; or
*—(C₂₋₁₀)alkylene-O—NH—;
T¹ represents
—C(O)—(C₀₋₁₀)alkylene-C(O)X;
—C(O)—CH₂CH₂—(OCH₂CH₂)ᵣ—C(O)X, wherein r is from 1 to 5;
—C(O)—CH₂(CH₂)_{f}(SCH₂(CH₂)_{f′})_{f″}—C(O)X, wherein f is 0 or 1, f′ is 0 or 1, and f″ is
1, 2, or 3; 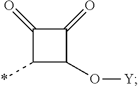
wherein w is 1 or 2; or 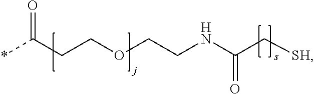
wherein j is from 1 to 4, preferably 1, and s is 1 or 2;
—C(O)X represents —C(O)OH or an activated ester, wherein preferably
X represents 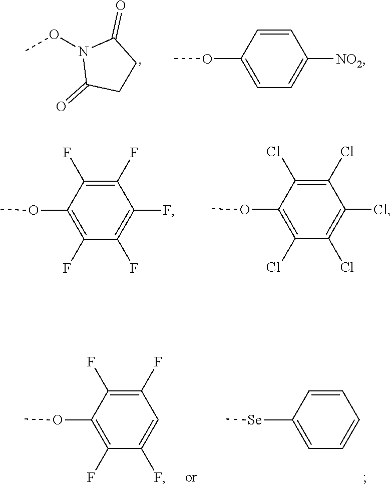
and
Y represents Me, Et, Bu or —(CH₂CH₂O)₃CH₃,
or a pharmaceutically acceptable salt thereof.

23. The oligosaccharide-carrier protein conjugate according to claim 1,
wherein
m is 4 or 5; and
n is 6 or 7;
or a pharmaceutically acceptable salt thereof.

24. The oligosaccharide-carrier protein conjugate according to claim 5, or a pharmaceutically acceptable salt thereof, wherein
-L-T-represents a linker L and a spacer T which together form a bridge which consists of a backbone which is a saturated chain counting from 5 to 25 atoms selected from the group consisting of carbon, nitrogen and oxygen, which chain may be unsubstituted or substituted with 1, 2, 3 or 4 substituents independently selected from oxo, (C₁₋₄)alkyl, fluoro and (C₁₋₂)alkoxy.

25. The oligosaccharide-carrier protein conjugate according to claim 6, or a pharmaceutically acceptable salt thereof, wherein the backbone of the bridge has a length of 8 to 16 atoms covalently linked together that forms the shortest distance between the oxygen at C1 of the reducing end of the oligosaccharide and the nitrogen of the amino group of a lysine residue at the carrier protein CRM₁₉₇.

26. The oligosaccharide-carrier protein conjugate according to claim 7, or a pharmaceutically acceptable salt thereof, wherein
L represents
*—(CH₂)ₗ—NH—; wherein l is from 2 to 6;
*—(CH₂CH₂O)ₐ—CH₂CH₂NH—, wherein a is 1 or 2; or
*—(CH₂)_{g}—O—NH—, wherein g is from 2 to 6; and
T represents
—C(O)—(CH₂)ₚ—C(O)—, wherein p is from 0 to 6;
—C(O)—CH₂CH₂—(OCH₂CH₂)ᵣ—C(O)—, wherein r is from 1 to 3;
—C(O)—CH₂ (CH₂)_{f}— (SCH₂ (CH₂)_{f′})_{f″}—C(O)—, wherein f is 0 or 1, f′ is 0 or 1, and f″ is 1; or

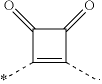

27. A pharmaceutical composition comprising, as active principle, an oligosaccharide-carrier protein conjugate according to claim 12, or a pharmaceutically acceptable salt thereof, and at least one therapeutically inert excipient.

28. The pharmaceutical composition according to claim 27, further comprising an adjuvant.

29. A pharmaceutical composition comprising, as active principle, an oligosaccharide-carrier protein conjugate according to claim 13, or a pharmaceutically acceptable salt thereof, and at least one therapeutically inert excipient.

30. The pharmaceutical composition according to claim 29, further comprising an adjuvant.

31. A method for prevention and/or treatment of a K. pneumoniae infection in a human and/or animal, wherein the method comprises administering an oligosaccharide-carrier protein conjugate according to claim 12, or a pharmaceutically acceptable salt thereof, to the human and/or animal.

32. The method according to claim 31, wherein the K. pneumoniae infection is selected from the group consisting of a K. pneumoniae infection in an individual of 50 years or older; a hospital acquired K. pneumoniae infection; a community-acquired K. pneumoniae infection; and pneumonia, bronchitis, meningitis, urinary tract infection, intra-abdominal infection, wound infection, infection of blood, osteomyelitis, bacteremia, septicemia, liver abscess, and inflammatory bowel disease (IBD), all caused by K. pneumoniae infection.

33. The method according to claim 32, wherein the hospital acquired K. pneumoniae infection is nosocomial pneumonia, a nosocomial bloodstream infection or a nosocomial urinary tract infection.

34. A method for prevention and/or treatment of a K. pneumoniae infection in a human and/or animal, wherein the method comprises administering an oligosaccharide-carrier protein conjugate according to claim 13, or a pharmaceutically acceptable salt thereof, to the human and/or animal.

35. The method according to claim 34, wherein the K. pneumoniae infection is selected from the group consisting of a K. pneumoniae infection in an individual of 50 years or older; a hospital acquired K. pneumoniae infection; a community-acquired K. pneumoniae infection; and pneumonia, bronchitis, meningitis, urinary tract infection, intra-abdominal infection, wound infection, infection of blood, osteomyelitis, bacteremia, septicemia, liver abscess, and inflammatory bowel disease (IBD), all caused by K. pneumoniae infection.

36. The method according to claim 35, wherein the hospital acquired K. pneumoniae infection is nosocomial pneumonia, a nosocomial bloodstream infection or a nosocomial urinary tract infection.

37. A multivalent vaccine comprising the oligosaccharide-carrier protein conjugate according to claim 12, or a pharmaceutically acceptable salt thereof.

38. A multivalent vaccine comprising the oligosaccharide carrier protein conjugate according to claim 13, or a pharmaceutically acceptable salt thereof.
